(19) United States
(12) Patent Application Publication
(10) Pub. No.: US 2005/0054829 A1
(43) Pub. Date: Mar. 10, 2005

(54) Compositions and methods relating to TSP-30a, b, c and d

(76) Inventors: Steven R. Wiley, Seattle, WA (US); Jalaleddin Vakili, Seattle, WA (US)

Correspondence Address: IMMUNEX CORPORATION;LAW DEPARTMENT, 1201 AMGEN COURT WEST, SEATTLE, WA, 98119, US

(21) Appl. No.: 10/883,144

(22) Filed: Jul. 1, 2004

Related U.S. Application Data
(60) Provisional application No. 60/489,409, filed on Jul. 22, 2003.

(51) Int. Cl. C12N 9/10; C07K 14/52

(52) U.S. Cl. 530/351

(57) ABSTRACT

The present invention provides compositions and methods relating to polynucleotides and polypeptides derived from the TSP-30a, b, c, and d genes. In particular embodiments the invention provides polynucleotides and polypeptides derived from human, mouse, and zebrafish TSP-30 genes, for example, a novel class of four exon splice variants.

This application claims the benefit of U.S. provisional application 60/489,409, filed Jul. 22, 2003, the disclosure of which is incorporated by reference.

### BACKGROUND

A large and growing number of proteins contain thrombospondin-1 (“TSP-1”) type 1 repeats (“TSRs”). See Tan et al., 2002, J. Cell Biol. 159:373-82. TSRs were originally recognized in human endothelial cell TSP-1, and subsequently found in other proteins, such as complement factors C8 and C9, the F-spondin gene family, the members of the semaphorin 5 family, UNC-5, SCO-spondin, and others. See Adams et al., 2000, Dev. Dyn. 218:280-99.

TSR-containing proteins are involved in a wide range of physiological processes. Many TSR-containing proteins are expressed during development, where some apparently play a role in the guidance of cell and growth cone migration, while others appear to be involved in regulating angiogenesis.

The three-dimensional structure of the TSR has been determined using X-ray crystallography. Tan et al., 2002, J. Cell Biol. 159:373-382. These studies revealed that the TSR adopts an antiparallel, three-stranded fold consisting of alternating stacked layers of tryptophan and arginine residues from respective strands, with disulfide bonds on each end. This structure has a grooved “front” face of exposed tryptophan and arginine residues that has been proposed to be the TSR's recognition domain, through which it interacts with other molecules, particularly negatively charged proteoglycans.

Small polypeptides derived from the TSRs of TSP-1 have been shown to be potent inhibitors of angiogenesis. In preclinical testing, these angiostatic peptides have shown promise as therapeutic agents in anti-angiogenic therapy.

Midkine (“MK”) and pleiotrophin (“PTN”) are two small TSR-containing growth factors that are found in a wide array of vertebrates, including fish, rodents and primates. See Muramatsu, 2002, J. Biochem. 132:359-71. Experimental evidence suggests that MK and PTN play a role in various developmental, inflammatory and tumorigenic processes, including angiogenesis. See id.

Thus, TSR-containing proteins are a therapeutically important group of molecules. Accordingly, there is a need in the art to identify other proteins that contain TSRs.

### SUMMARY OF THE INVENTION

We have identified novel proteins and polynucleotides encoded by a family of genes found in organisms as diverse as human, mice and zebrafish, which we have named the TSP-30a, b, c, and d proteins (collectively, “the TSP-30 proteins”). The TSP-30 proteins are small, extracellular proteins that contain TSRs and share significant sequence similarity with each other, with MK and PTN, and with the extracellular domain of TSP-1. Certain polynucleotide and polypeptide sequences derived from some of these genes are described in, e.g., US App. Pub. No. 2002065394, European App. Pub. No. EP 1074617, PCT publications WO 2002079398, WO 2001077169, WO 2001057190, WO 2001057188, WO 2001040294, WO 9849302, WO 03029437, and WO 02070539. Novel huTSP-30 polypeptides, polynucleotides, antibodies, compositions, kits, and methods are provided herein.

In one aspect, the present invention provides an isolated polypeptide comprising amino acid residues 213 through 235 of huTSP-30a 4ex as they are numbered in FIG. 13, wherein said polypeptide has a biological activity of huTSP-30a 4ex.

In another aspect, the present invention provides an isolated polypeptide comprising a sequence of at least 15 contiguous amino acid residues of residues 1 through 212 of the sequence of huTSP-30a as they are numbered in FIG. 13, wherein said polypeptide has a biological activity of huTSP-30a 4ex and does not comprise the sequence of amino acid residues 213 through 272 of huTSP-30a as they are numbered in FIG. 13. In one embodiment, said polypeptide comprises a sequence of at least 20 contiguous amino acid residues 1 through 212 of the sequence of huTSP30a as they are numbered in FIG. 13. In another embodiment, said polypeptide comprises a sequence of at least 25 contiguous amino acid residues 1 through 212 of the sequence of huTSP30a as they are numbered in FIG. 13. In another aspect, the present invention provides an isolated polypeptide comprising a sequence of amino acids that is at least 90% identical to the sequence of huTSP-30a 4ex as they are numbered in FIG. 13, wherein said polypeptide has a biological activity of huTSP-30a 4ex. In one embodiment, said polypeptide comprises a sequence of amino acids that is at least 95% identical to the sequence of huTSP-30a 4ex as they are numbered in FIG. 13. In another embodiment, said polypeptide comprises a sequence of amino acids that is at least 98% identical to the sequence of huTSP-30a 4ex as they are numbered in FIG. 13. In another embodiment, said polypeptide comprises the sequence of amino acids of huTSP-30a 4ex as they are numbered in FIG. 13.

In another aspect, the present invention provides an isolated polypeptide comprising a sequence of amino acids that is encoded by a nucleic acid that hybridizes under moderately stringent conditions to a nucleic acid comprising the complement of the highlighted portion of the nucleotide sequence of huTSP-30a 4ex they are numbered in FIG. 2, wherein said polypeptide has a biological activity of huTSP-30a 4ex.

In another aspect, the present invention provides an isolated polypeptide that binds an antibody that binds huTSP-30a 4ex.

In another aspect, the present invention provides an isolated polypeptide comprising an amino acid sequence selected from the group consisting of residues 225 through 234 of huTSP-30b1 as they are numbered in FIG. 14 and residues 199 through 266 pfhuTSP-30b 4ex as they are numbered in FIG. 14, wherein said polypeptide has a biological activity of huTSP-30a 4ex.

In another aspect, the present invention provides an isolated polypeptide comprising a sequence of at least 15 contiguous amino acid residues of residues 1 through 198 of the sequence of huTSP-30b as they are numbered in FIG. 13, wherein said polypeptide has a biological activity of huTSP-30b 4ex and does not comprise the sequence of amino acid residues 199 through 224 of huTSP-30b as they are numbered in FIG. 14. In one embodiment, the isolated polypeptide of claim 12 wherein said polypeptide comprises a sequence of at least 20 contiguous amino acid residues 1 through 198 of the sequence of huTSP30b as they are numbered in FIG. 14. In another embodiment, said polypeptide comprises a sequence of at least 25 contiguous amino acid residues 1 through 198 of the sequence of huTSP30b as they are numbered in FIG. 14.

In another aspect, the present invention provides an isolated polypeptide comprising a sequence of amino acids that is at least 90% identical to a sequence selected from the group consisting of huTSP-30b1 as shown in FIG. 14, and huTSP-30b 4ex as shown in FIG. 14, wherein said polypeptide has a biological activity of huTSP-30 4ex or of huTSP-30b1 and does not consist of a sequence that is identical to huTSP-30b as shown in FIG. 14. In one embodiment, said polypeptide comprises a sequence of amino acids that is at least 95% identical to a sequence selected from the group consisting of huTSP-30b1 as shown in FIG. 14, and huTSP-30b 4ex as shown in FIG. 14. In another embodiment, said polypeptide comprises a sequence of amino acids that is at least 98% identical to a sequence selected from the group consisting of huTSP-30b1 as shown in FIG. 14, and huTSP-30b 4ex as shown in FIG. 14. In another embodiment, said polypeptide comprises the sequence of amino acids of huTSP-30b1 or of huTSP-30b 4ex as shown in FIG. 14.

In another aspect, the present invention provides an isolated polypeptide comprising a sequence of amino acids that is encoded by a nucleic acid that hybridizes under moderately stringent conditions to a nucleic acid comprising the complement of a nucleotide sequence selected from the group consisting of the highlighted portion of the nucleotide sequence of huTSP-30b1 as shown in FIG. 2, and the highlighted portion of the nucleotide sequence of huTSP-30b 4ex shown in FIG. 2, wherein said polypeptide has a biological activity of huTSP-30b1 or of huTSP-30b 4ex.

In another aspect, the present invention provides an isolated polypeptide that binds an antibody that binds to huTSP-30b 4ex.

In another aspect, the present invention provides an isolated polypeptide comprising amino acid residues 207 through 242 of huTSP-30c 4ex as they are numbered in FIG. 15, wherein said polypeptide has a biological activity of huTSP-30c 4ex.

In another aspect, the present invention provides an isolated polypeptide comprising a sequence of at least 15 contiguous amino acid residues of a sequence selected from the group consisting of residues 1 through 206 of the sequence of huTSP-30c as they are numbered in FIG. 15, residues 1 through 206 of the sequence of huTSP-30c1 as they are numbered in FIG. 15, residues 1 through 206 of the sequence of huTSP-30c2 as they are numbered in FIG. 15, residues 1 through 206 of the sequence of huTSP-30c3 as they are numbered in FIG. 15, and residues 1 through 206 of the sequence of huTSP-30c 4ex as they are numbered in FIG. 15, wherein said polypeptide has a biological activity of huTSP-30c, huTSP-30c1, huTSP-30c2, huTSP-30c3, or huTSP-30c 4ex and does not comprise the sequence of amino acid residues 207 through 243 of huTSP-30c as shown in FIG. 15. In one embodiment, said polypeptide comprises a sequence of at least 20 contiguous amino acid residues of a sequence selected from the group consisting of residues 1 through 206 of the sequence of huTSP-30c as they are numbered in FIG. 15, residues 1 through 206 of the sequence of huTSP-30c1 as they are numbered in FIG. 15, residues 1 through 206 of the sequence of huTSP-30c2 as they are numbered in FIG. 15, residues 1 through 206 of the sequence of huTSP-30c3 as they are numbered in FIG. 15, and residues 1 through 206 of the sequence of huTSP-30c 4ex as they are numbered in FIG. 15. In another embodiment, said polypeptide comprises a sequence of at least 25 contiguous amino acid residues of a sequence selected from the group consisting of residues 1 through 206 of the sequence of huTSP-30c as they are numbered in FIG. 15, residues 1 through 206 of the sequence of huTSP-30c1 as they are numbered in FIG. 15, residues 1 through 206 of the sequence of huTSP-30c2 as they are numbered in FIG. 15, residues 1 through 206 of the sequence of huTSP-30c3 as they are numbered in FIG. 15, and residues 1 through 206 of the sequence of huTSP-30c 4ex as they are numbered in FIG. 15.

In another aspect, the present invention provides an isolated polypeptide comprising a sequence of amino acids that is at least 90% identical to a sequence selected from the group consisting of huTSP-30c3 as shown in FIG. 15, and huTSP-30c 4ex as shown in FIG. 15, wherein said polypeptide has a biological activity of huTSP-30c 4ex or of huTSP-30c3 and does not consist of a sequence that is identical to huTSP-30c, huTSP-30c1, or huTSP-30c2 as shown in FIG. 14. In one embodiment, said polypeptide comprises a sequence of amino acids that is at least 95% identical to a sequence selected from the group consisting of huTSP-30c3 as shown in FIG. 15, and huTSP-30c 4ex as shown in FIG. 15. In another embodiment, said polypeptide comprises a sequence of amino acids that is at least 98% identical to a sequence selected from the group consisting of huTSP-30c3 as shown in FIG. 15, and huTSP-30c 4ex as shown in FIG. 15. In another embodiment, said polypeptide comprises the sequence of amino acids of huTSP-30c3 or of huTSP-30c 4ex as shown in FIG. 15.

In another aspect, the present invention provides an isolated polypeptide comprising a sequence of amino acids that is encoded by a nucleic acid that hybridizes under moderately stringent conditions to a nucleic acid comprising the complement of a nucleotide sequence selected from the group consisting of the highlighted portion of the nucleotide sequence of huTSP-30c3 as shown in FIG. 2, and the highlighted portion of the nucleotide sequence of huTSP-30c 4ex shown in FIG. 2, wherein said polypeptide has a biological activity of huTSP-30c3 or of huTSP-30c 4ex and does not comprise the sequence of huTSP-30c, huTSP-30c1, huTSP-30c2, huTSP-30c frag1, or huTSP-30c frag2, as shown in FIG. 15.

In another aspect, the present invention provides a polypeptide that binds an antibody that binds huTSP-30c3 or huTSP-30c 4ex.

In another aspect, the present invention provides an isolated polypeptide comprising amino acid residues 210 through 292 of huTSP-30d 4ex as they are numbered in FIG. 16, wherein said polypeptide has a biological activity of huTSP-30d 4ex.

In another aspect, the present invention provides an isolated polypeptide comprising a sequence of at least 15 contiguous amino acid residues of a sequence selected from the group consisting of residues 1 through 209 of the sequence of huTSP-30d as they are numbered in FIG. 16, residues 1 through 209 of the sequence of huTSP-30d1 as they are numbered in FIG. 16, residues 1 through 209 of the sequence of huTSP-30d 4ex as they are numbered in FIG. 16, residues 28 through 209 of the sequence of huTSP-30d frag1 as they are numbered in FIG. 16, and residues 33 through 209 of the sequence of huTSP-30d frag2 as they are numbered in FIG. 15, wherein said polypeptide has a biological activity of huTSP-30d, huTSP-30d1, huTSP-30d 4ex, huTSP-30d frag1, or huTSP-30d frag2 and does not comprise the sequence of amino acid residues 210 through 292 of huTSP-30d as shown in FIG. 16. In one embodiment, said polypeptide comprises a sequence of at least 20 contiguous amino acid residues of a sequence selected from the group consisting of residues 1 through 209 of the sequence of huTSP-30d as they are numbered in FIG. 16, residues 1 through 209 of the sequence of huTSP-30d1 as they are numbered in FIG. 16, residues 1 through 209 of the sequence of huTSP-30d 4ex as they are numbered in FIG. 16, residues 28 through 209 of the sequence of huTSP-30d frag1 as they are numbered in FIG. 16, and residues 33 through 209 of the sequence of huTSP-30d frag2 as they are numbered in FIG. 15. In another embodiment, said polypeptide comprises a sequence of at least 25 contiguous amino acid residues of a sequence selected from the group consisting of residues 1 through 209 of the sequence of huTSP-30d as they are numbered in FIG. 16, residues 1 through 209 of the sequence of huTSP-30d1 as they are numbered in FIG. 16, residues 1 through 209 of the sequence of huTSP-30d 4ex as they are numbered in FIG. 16, residues 28 through 209 of the sequence of huTSP-30d frag1 as they are numbered in FIG. 16, and residues 33 through 209 of the sequence of huTSP-30d frag2 as they are numbered in FIG. 15.

In another aspect, the present invention provides an isolated polypeptide comprising a sequence of amino acids that is at least 90% identical to huTSP-30d 4ex as shown in FIG. 16 wherein said polypeptide has a biological activity of huTSP-30d 4ex. In one embodiment, said polypeptide comprises a sequence of amino acids that is at least 95% identical to huTSP-30d 4ex as shown in FIG. 16. In another embodiment, said polypeptide comprises a sequence of amino acids that is at least 98% identical to huTSP-30d 4ex as shown in FIG. 16. In another embodiment, said polypeptide comprises the sequence of amino acids of huTSP-30d 4ex as shown in FIG. 16.

In another aspect, the present invention provides an isolated polypeptide comprising a sequence of amino acids that is encoded by a nucleic acid that hybridizes under moderately stringent conditions to a polynucleotide comprising the nucleotide sequence of huTSP-30d 4ex as shown in FIG. 2 wherein said polypeptide has a biological activity of huTSP-30d 4ex and does not comprise the sequence of huTSP-30d, huTSP-30d1, huTSP-30d frag1, or huTSP-30d frag2, as shown in FIG. 16.40.

In another aspect, the present invention provides an isolated polypeptide that binds an antibody that binds huTSP-30d 4ex.

In another aspect, the present invention provides an isolated polypeptide comprising a sequence selected from the group consisting of a leader sequence as shown in FIG. 13, 14, 15, or 16, an N-terminal heparin binding cluster as shown in FIG. 13, 14, 15, or 16, a cysteine repeat as shown in FIG. 13, 14, 15, or 16, and a thrombospondin repeat as shown in FIG. 13, 14, 15, or 16, wherein said polypeptide does not comprise a C-terminal heparin binding cluster as shown in FIG. 13, 14, 15, or 16.

In another aspect, the present invention provides an isolated polypeptide comprising a sequence selected from the group consisting of a leader sequence as shown in FIG. 13, 14, 15, or 16, an N-terminal heparin binding cluster as shown in FIG. 13, 14, 15, or 16, a cysteine repeat as shown in FIG. 13, 14, 15, or 16, and a thrombospondin repeat as shown in FIG. 13, 14, 15, or 16, wherein said polypeptide inhibits huTSP-30a, huTSP-30b, huTSP-30c, or huTSP-30d.

In another aspect, the present invention provides an isolated polypeptide that comprises at least two cysteine repeats and at least one thrombospondin repeat. In one embodiment, said polypeptide comprises two cysteine repeats and one thrombospondin repeat. In another embodiment, said polypeptide comprises at least two thrombospondin domains.

In another aspect, the present invention provides an isolated polypeptide that comprises an oligomerization domain. In one embodiment, said oligomerization domain comprises an Fc domain or a leucine zipper domain.

In another aspect, the present invention provides an isolated polynucleotide comprising a sequence that encodes a polypeptide as described above. In one embodiment, a vector comprises said polynucleotide. In another embodiment said vector is an expression vector. In another embodiment, a cell comprises said expression vector. In another embodiment, the invention provides a method of expressing a polypeptide comprising incubating said cell under conditions that allow expression of said polynucleotide.

In another aspect, the present invention provides an isolated molecule that binds to a polypeptide as described above. In one embodiment, said molecule comprises an antibody. In another embodiment, said antibody is a monoclonal antibody. In another embodiment, said antibody is a human, humanized, or chimeric antibody. In another embodiment, said fragment comprises a Fab fragment, an Fc fragment, an scFv, a variable region, or a complementarity determining region of an antibody. In another embodiment, said molecule comprises a soluble fragment of a receptor for a polypeptide selected from the group consisting of huTSP-30a 4ex, huTSP-30b1, huTSP-30b 4ex, huTSP-30c3, huTSP-30c 4ex, or huTSP-30d 4ex.

In another aspect, the present invention provides a pharmaceutical composition comprising a polypeptide as described above and a pharmaceutically acceptable diluent, buffer, or excipient.

In another aspect, the present invention provides a method of treating a condition in a subject comprising administering to said subject a substance selected from the group consisting of a polypeptide described above, a polynucleotide described above, and a molecule described above. In one embodiment, said condition is a cancerous condition. In another embodiment, said cancerous condition is a cancer of a tissue of the nervous system. In another embodiment, said cancerous condition is a cancer of the brain. In another embodiment, said cancerous condition is a cancer of the spinal cord. In another embodiment, said cancerous condition is a cancer of the skin. In another embodiment, said cancerous condition is a cancer of a tissue of the reproductive system. In another embodiment, said cancerous condition is a cancer of the ovaries. In another embodiment, said cancerous condition is a cancer of the uterus. In another embodiment, said cancerous condition is a cancer of the testis. In another embodiment, said cancerous condition is a cancer of a tissue of the gastrointestinal tract. In another embodiment, said cancerous condition is a cancer of the stomach. In another embodiment, said cancerous condition is a cancer of the small intestine. In another embodiment, said cancerous condition is a cancer of the colon. In another embodiment, said cancerous condition is a cancer of the lungs. In another embodiment, said cancerous condition is a cancer of the breast. In another embodiment, said cancerous condition is a cancer of the prostate. In another embodiment, said cancerous condition is a cancer of the skeletal muscle. In another aspect, said condition is an inflammatory condition. In another embodiment, said inflammatory condition is an inflammatory condition of a tissue of the nervous system. In another embodiment, said inflammatory condition is an inflammatory condition of the brain. In another embodiment, said inflammatory condition is an inflammatory condition of the spinal cord. In another embodiment, said inflammatory condition is an inflammatory condition of the skin. In another embodiment, said inflammatory condition is an inflammatory condition of a tissue of the reproductive system. In another embodiment, said inflammatory condition is an inflammatory condition of the ovaries. In another embodiment, said inflammatory condition is an inflammatory condition of the uterus. In another embodiment, said inflammatory condition is an inflammatory condition of the testis. In another embodiment, said inflammatory condition is an inflammatory condition of a tissue of the gastrointestinal tract. In another embodiment, said inflammatory condition is an inflammatory condition of the stomach. In another embodiment, said inflammatory condition is an inflammatory condition of the small intestine. In another embodiment, said inflammatory condition is an inflammatory condition of the colon. In another embodiment, said inflammatory condition is an inflammatory condition of the lungs. In another embodiment, said inflammatory condition is an inflammatory condition of the breast. In another embodiment, said inflammatory condition is an inflammatory condition of the prostate. In another embodiment, said inflammatory condition is an inflammatory condition of the skeletal muscle.

In another aspect, the present invention provides a method of determining whether a tissue is cancerous, comprising determining whether said tissue has more of a polypeptide comprising the sequence of huTSP-30a, huTSP-30a 4ex, huTSP-30b, huTSP-30b1, huTSP-30b 4ex, huTSP-30c, huTSP-30c1, huTSP-30c2, huTSP-30c3, huTSP-30c 4ex, huTSP-30c frag1, huTSP-30c frag2, huTSP-30d, huTSP-30d1, huTSP-30d 4ex, huTSP-30d frag1, or huTSP-30d frag2, than a non-cancerous control tissue, wherein more of said polypeptide in said tissue than in said non-cancerous control tissue indicates that said tissue is cancerous.

In another aspect, the present invention provides a method of determining whether a tissue is cancerous, comprising determining whether said tissue has more of a polynucleotide that encodes the amino acid sequence of huTSP-30a, huTSP-30a 4ex, huTSP-30b, huTSP-30b1, huTSP-30b 4ex, huTSP-30c, huTSP-30c1, huTSP-30c2, huTSP-30c3, huTSP-30c 4ex, huTSP-30c frag1, huTSP-30c frag2, huTSP-30d, huTSP-30d1, huTSP-30d 4ex, huTSP-30d frag1, or huTSP-30d frag2, than a non-cancerous control tissue, wherein more of said polynucleotide in said tissue than in said non-cancerous control tissue indicates that said tissue is cancerous.

### BRIEF DESCRIPTION OF THE DRAWINGS

FIG. 1 presents an alignment of huTSP-30a, huTSP-30b, huTSP-30c and huTSP-30d-derived amino acid sequences. Identical residues are shaded in black. Similar residues are shaded in gray. Dashes represent gaps introduced into the sequence to improve the alignment.

FIG. 2 presents polynucleotide sequences derived from huTSP-30a, huTSPP-30b, huTSP-30c and huTSP-30d. Coding sequences are in bold.

FIG. 3 presents an alignment of muTSP-30a, muTSP-30c and muTSP-30d-derived amino acid sequences. Identical residues are shaded in black. Similar residues are shaded in gray. Dashes represent gaps introduced into the sequence to improve the alignment.

FIG. 4 presents polynucleotide sequences derived from muTSP-30a, muTSP-30c and muTSP-30d. Coding sequences are in bold.

FIG. 5 presents an alignment of drTSP-30a, drTSP-30c and drTSP-30d-derived amino acid sequences. Identical residues are shaded in black. Similar residues are shaded in gray. Dashes represent gaps introduced into the sequence to improve the alignment.

FIG. 6 presents polynucleotide sequences derived from drTSP-30a, drTSP-30c and drTSP-30d. Coding sequences are in bold.

FIG. 7 presents an alignment of the TSR domains of huTSP-30a, b, c, and d against the TSR domains of various TSR-domain containing proteins. Identical residues are shaded in black. Similar residues are shaded in grey.

FIG. 8 presents a schematic diagram of the domain structure of the TSP-30 proteins. The domains are presented as boxes in the order they occur in the TSP-30 proteins, with the N-terminus at the left and the C-terminus at the right. Horizontal lines=leader sequence, vertical lines=cysteine repeat domains, wavy lines=TSR domain, stipples=heparin binding cluster, C=other conserved cysteine residues.

FIG. 9 presents an alignment of huTSP-30a, huTSP-30a 4ex, muTSP-30a, and drTSP-30a-derived amino acid sequences. Identical residues are shaded in black. Similar residues are shaded in gray. Dashes represent gaps introduced into the sequence to improve the alignment.

FIG. 10 presents an alignment of huTSP-30b and huTSP-30b 4ex-derived amino acid sequences. Identical residues are shaded in black. Similar residues are shaded in gray. Dashes represent gaps introduced into the sequence to improve the alignment.

FIG. 11 presents an alignment of huTSP-30c, huTSP-30c 4ex, muTSP-30c and drTSP-30c-derived amino acid sequences. Identical residues are shaded in black. Similar residues are shaded in gray. Dashes represent gaps introduced into the sequence to improve the alignment.

FIG. 12 presents an alignment of huTSP-30d, huTSP-30d 4ex, muTSP-30d and drTSP-30d amino acid sequences. Identical residues are shaded in black. Similar residues are shaded in gray. Dashes represent gaps introduced into the sequence to improve the alignment.

FIG. 13 presents an alignment of huTSP-30a-derived sequences. huTSP-30a corresponds to a sequence disclosed in PCT Pub. No. WO 01057190. Internal dashes represent gaps introduced into the sequences to improve their alignment. Residues and gaps in bold are unique to huTSP-30a 4ex, e.g., the absence of residues 213 through 272 of huTSP-30a, and the presence of residues 213 through 235 of huTSP-30a 4ex, as the residues are numbered in FIG. 13. Residues 1 through about 25 comprise a leader sequence; residues about 25 through about 32 comprise an N-terminal heparin binding cluster; residues about 44 through about 82 comprise a cysteine repeat; residues about 102 through about 130 comprise a cysteine repeat; residues about 148 through about 207 comprise a TSR, residues about 211 through 230 of huTSP-30a comprise C-terminal heparin binding clusters, as the residues are numbered in FIG. 13.

FIG. 14 presents an alignment of huTSP-30b-derived sequences. huTSP-30b corresponds to genbank sequence gi:14627121. Residues in bold represent sequences unique to huTSP-30b1 and/or huTSP-30 4ex, e.g., the absence of residues 198 through 224 of huTSP-30b, the presence of residues 225 through 234 of huTSP-30b1, and the presence of residues 198 through 266 of huTSP-30b 4ex, as the residues are numbered in FIG. 14. Residues 1 through about 21 comprise a leader sequence; residues about 22 through about 26 comprise an N-terminal heparin binding cluster; residues about 37 through about 75 comprise a cysteine repeat; residues about 95 through about 123 comprise a cysteine repeat; residues about 139 through about 197 comprise a TSR, residues about 204 through 218 of huTSP-30b and huTSP-30b1 comprise C-terminal heparin binding clusters, as the residues are numbered in FIG. 14.

FIG. 15 presents an alignment of huTSP-30c-derived polypeptide sequences. huTSP-30c corresponds to gsp:ABG76508 (PCT Pub. No. WO 2002060942) and huTSP-30c2 comprises polymorphism NCBI SNP Cluster I.D. No. rs859541. huTSP-30c frag1 (gi:20380783; Strausberg et al., 2002, Proc. Natl. Acad. Sci. USA 99:16899-16903) and huTSP-30c frag2 (gi:29735291; predicted from NCBI contig NT_{—}008046 by the International Human Genome Sequencing Consortium using automated computational analysis) are huTSP-30c-derived fragments. Dashes represent gaps introduced into the sequences to improve their alignment. Residues and gaps in bold are unique to huTSP-30c1, huTSP-30c2, huTSP-30c3, and/or huTSP-30c 4ex; e.g., the absence of residues 32 through 39 of huTSP-30c, the absence of residue 143 of huTSP-30c, the absence of residues 207 through 243 of huTSP-30c, and the presence of residues 207 through 242 of huTSP-30c 4ex, all sequences numbered as in FIG. 15. The L residue at position 185 of huTSP-30c2 is unique in a full-length huTSP-30c derived polypeptide. Residues 1 through about 24 comprise a leader sequence; residues about 25 through about 31 comprise an N-terminal heparin binding cluster; residues about 42 through about 80 comprise a cysteine repeat; residues about 101 through about 129 comprise a cysteine repeat; residues about 145 through about 204 comprise a TSR, residues about 207 through 223 of huTSP-30c, huTSP-30c1, huTSP-30c2, huTSP-30c3, huTSP-30 frag1, and huTSP-30 frag2 comprise C-terminal heparin binding clusters, as the residues are numbered in FIG. 15.

FIG. 16 presents an alignment of huTSP-30d-derived sequences. The sequence of huTSP-30d 4ex differs from that of huTSP-30d (WO 03029437), huTSP-30d1 (PCT Pub. No. WO 03029405), huTSP-30d frag1 (gi: 27480552 determined by the International Human Genome Sequencing Consortium), and huTSP-30d frag2 (Celera gene prediction hCP1740058) in that it is encoded by a four-exon splice product of the gene rather than a five-exon splice product. Residues in bold are unique to huTSP-30d 4ex with respect to huTSP-30d, huTSP-30d1, huTSP-30d frag1 and/or huTSP-30d frag2, e.g., residues 210 through 233 of huTSP-30d 4ex as it is numbered in FIG. 16. Underlined residues and gaps in huTSP-30d1, huTSP-30d frag1 and huTSP-30d frag2 are unique in those sequences as compared to huTSP-30d. Residues 1 through about 24 comprise a leader sequence; residues about 25 through about 31 comprise an N-terminal heparin binding cluster; residues about 43 through about 80 comprise a cysteine repeat; residues about 102 through about 130 comprise a cysteine repeat; residues about 148 through about 207 comprise a TSR, residues about 211 through 243 of huTSP-30d, huTSP-30d1, huTSP-30d frag1, and huTSP-30d frag2 comprise C-terminal heparin binding clusters, as the residues are numbered in FIG. 16.

### DETAILED DESCRIPTION OF THE INVENTION

Proteins of the Invention

Domain Structure of the TSP-30 Proteins

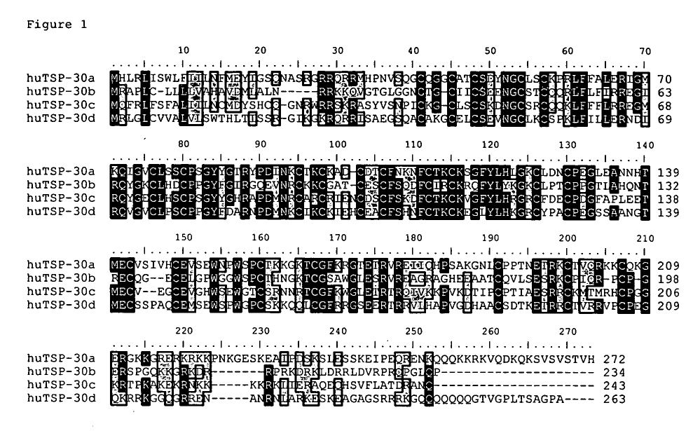

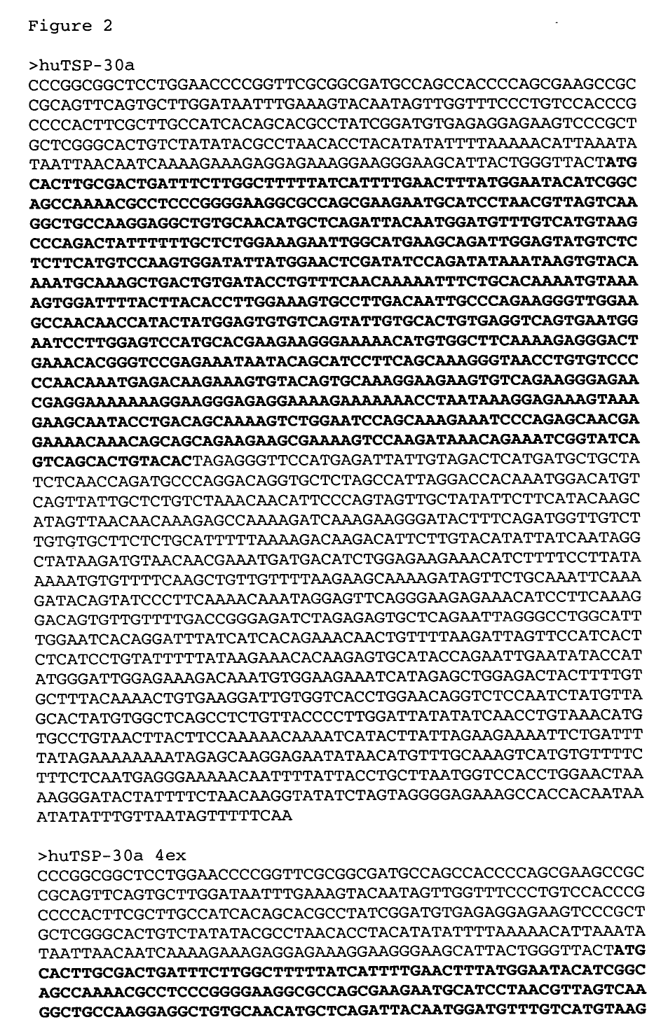

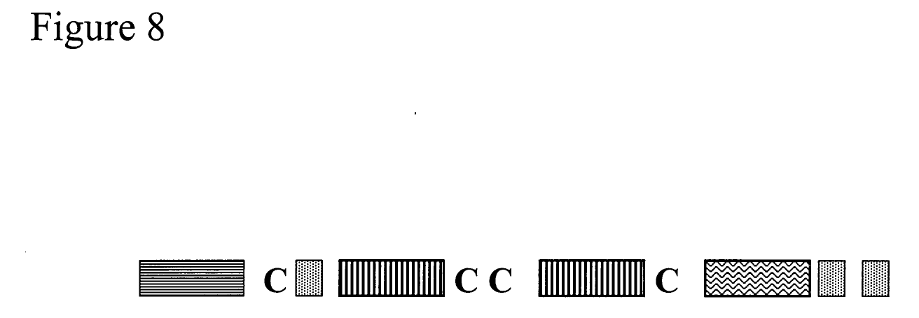

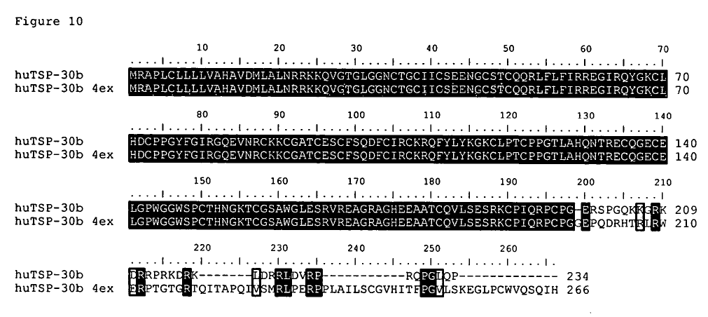

Human TSP-30a, b, c and d (“huTSP-30a, b, c and d”), mouse TSP-30a, b, c and d (“muTSP-30a, b, c, and d”) and zebrafish TSP-30a, b, c, and d (“drTSP-30, a, b, c and d”), and variants thereof, have the amino acid sequences and are encoded by the nucleotide sequences shown in FIGS. 1-6 and 9-16. Each TSP-30 amino acid sequence comprises an ordered series of domains and sequence features as illustrated in FIG. 8. Each TSP-30 amino acid sequence comprises an N-terminal leader sequence extending from residue 1 to about residue 25. This sequence can act as a signal sequence, causing the polypeptide to be secreted from a cell expressing it into the extracellular space. The signal peptide cleavage site for the TSP-30a, b, c and d polypeptides can be predicted using a computer algorithm. However, one of skill in the art will recognize that the cleavage site of the signal sequence may vary depending upon a number of factors including the cell or organism in which the polypeptide is expressed. Accordingly, the N-terminus of a mature form of a TSP-30 polypeptide of the invention may vary by 1, 2, 3, 4, 5, 6, 7, 8, 9, or 10 amino acid residues on either side of residue 25.

Alternatively, the mature form of a polypeptide of the invention can comprise the N-terminal leader sequence. In one such embodiment, the N-terminal sequence acts as a type II transmembrane domain, anchoring the polypeptide to a cell such that the polypeptide's C-terminal portion is in the extracellular space and its N-terminal portion spans the cell's cytoplasmic membrane. In this embodiment, the polypeptide of the invention and the N-terminal sequence remain attached to each other via a peptide bond, such that the polypeptide remains integrally associated with the extracellular membrane, or are cleaved from each other, e.g., by a protease. After cleavage, the C-terminal portion of the polypeptide can remain attached to the N-terminal portion of the polypeptide, for example, through a disulfide bond, salt bridge, hydrogen bond, hydrophobic interaction, and/or other covalent or non-covalent bond, can remain associated with the cell via an interaction with another molecule associated with the cell's membrane (e.g., a component of the extracellular matrix or another transmembrane protein), or the polypeptide can be released into the extracellular milieu. Whether an N-terminal sequence acts as a signal sequence that is cleaved during secretion or a type II transmembrane domain can depend on the type of cell that it is expressed in.

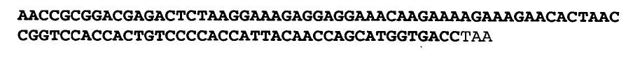

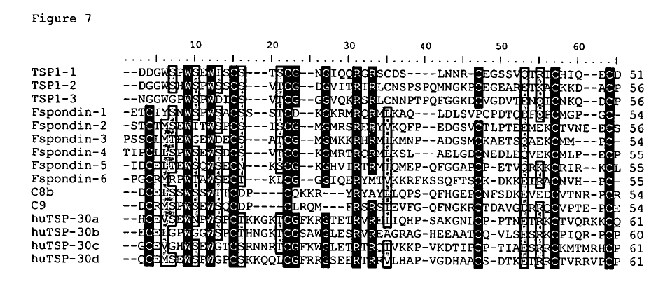

TSP-30a, b, c, and d further comprise a TSP-like region (“TSR”), as shown in FIGS. 1, 3 and 5. The three-dimensional structure of the TSR has been elucidated using X-ray crystallography. Tan et al., 2002, J. Cell Biol. 159:373-382. These studies revealed that the TSR adopts an antiparallel, three-stranded fold consisting of alternating stacked layers of tryptophan and arginine residues from respective strands, with disulfide bonds on each end. This structure has a grooved “front” face of exposed tryptophan and arginine residues that has been proposed to be the TSR's recognition domain, through which it interacts with other molecules, particularly negatively charged proteoglycans. See id. The cysteine, tryptophan and arginine residues that are conserved between TSRs and that form the folded structure are found in the TSP-30 proteins (FIG. 7). Thus, without being bound to a particular theory, it is likely that the TSRs found in the TSP-30s adopt this conformation and interact with negatively charged molecules in the extracellular matrix, for example, proteoglycans. Interestingly, the TSR domain of MK, which has less similarity to the TSP-1 TSR domains than the TSP-30 proteins have, adopts a three stranded antiparallel β sheet conformation that is reminiscent of the three-stranded fold of the TSP-1 TSR domains, but it lacks their ordered cysteine-tryptophan-arginine layered structure. Tan et al., 2002, J. Cell Biol. 159:373-82.

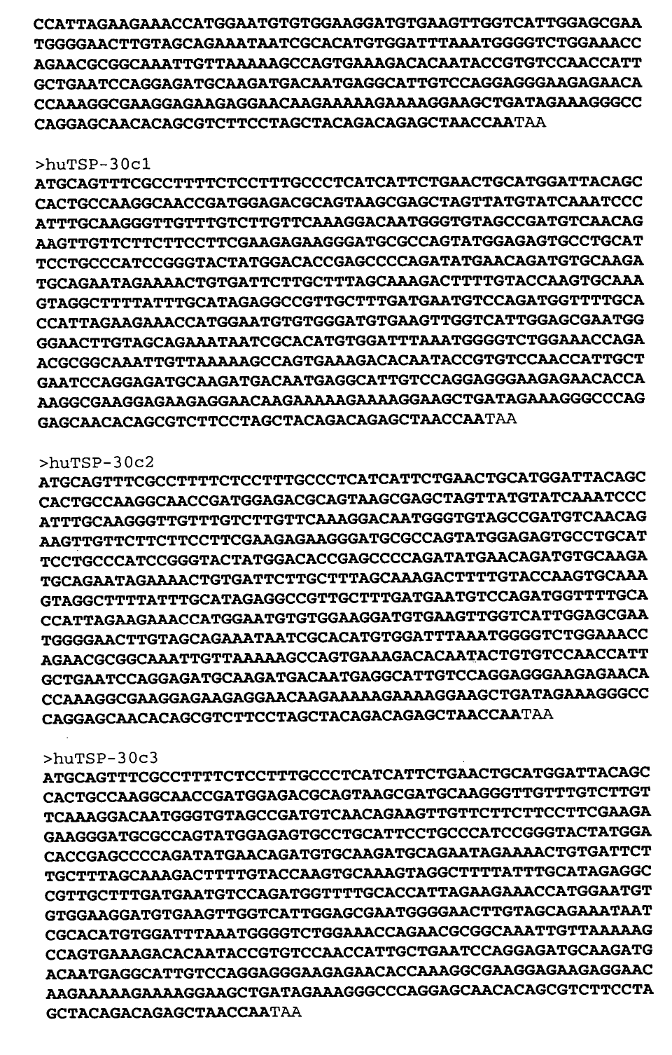

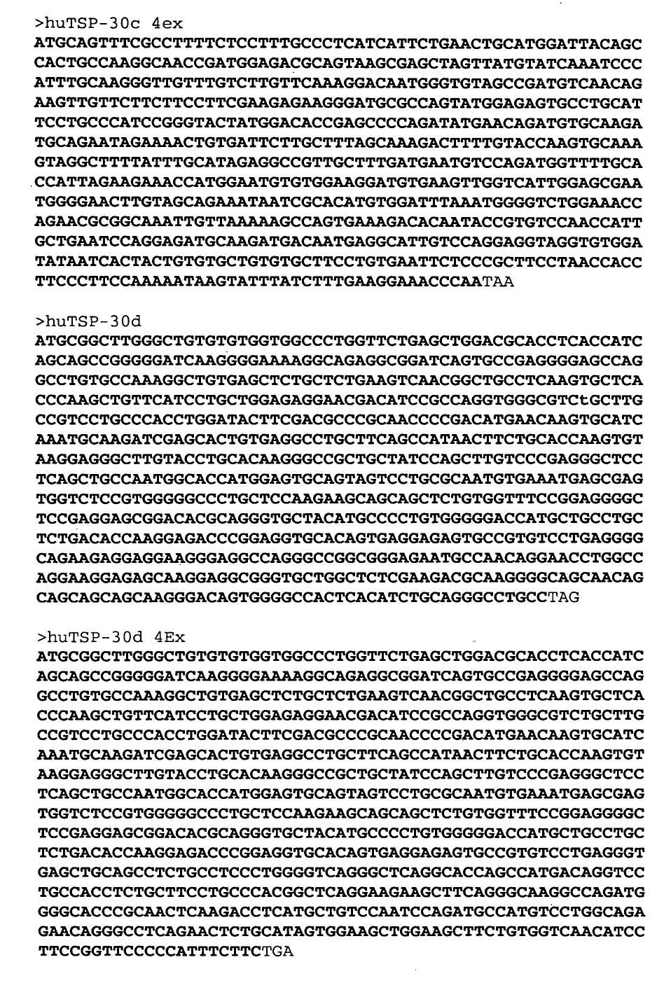

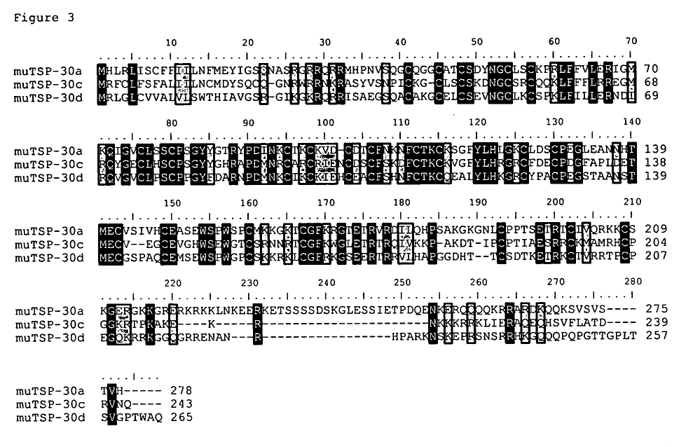

The TSP-30 polypeptides further comprise a series of conserved cysteine residues, as shown in FIGS. 1, 3, and 5. The huTSP-30 and muTSP-30 polypeptides each comprises 22 conserved cysteine residues (FIGS. 1 and 3), while the drTSP-30 polypeptides each comprises 20-22 conserved residues (FIG. 5). These cysteine residues are organized into discreet domains, as shown in FIG. 8. Particularly noteworthy are two domains that each comprises six conserved cysteine residues. These cysteine repeat domains are similar to a domain found in MK and PTN. Like the TSP-30 proteins, MK and PTN comprise an N-terminal cysteine repeat domain featuring six conserved cysteine residues and a C-terminal TSR domain. Muramatsu, 2002, J. Biochem. 132:359-71. The solution structure of MK has been determined using NMR spectroscopy. Iwasaki et al., 1997, EMBO J. 16:6936-46. The conserved cysteine repeat domain and the TSR domain of MK each adopts a three stranded antiparallel β sheet conformation. Thus, without being bound to a particular theory, it is likely that the two conserved cysteine domains of the TSP-30 proteins each adopts a three stranded antiparallel β sheet formation.

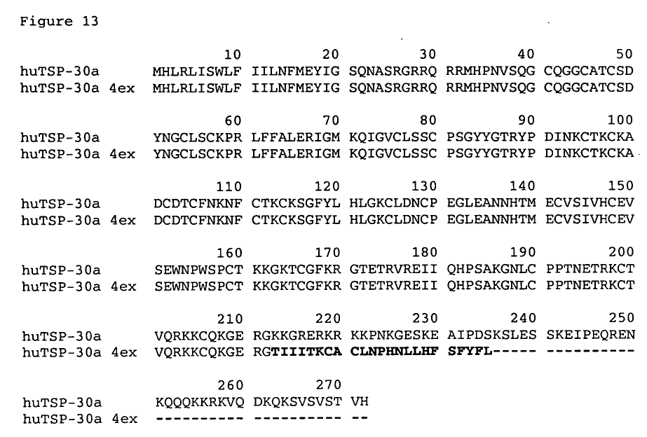

The TSP-30 proteins further comprise three heparin binding clusters, one near the N-terminus and two near the C-terminus (FIGS. 1, 3, 5 and 8). Heparin clusters are groups of positively charged residues that interact with heparin sulfate, a negatively charged polysaccharide covalently attached to select polypeptides of the extracellular matrix. MK, which, as explained above, has a domain structure that is similar to that of the TSP-30 proteins, comprises heparin binding clusters and binds to heparin sulfate on a number of proteins via its TSR domain. Thus, without being bound to a particular theory, it is likely that the TSP-30 proteins of the present invention bind to one or more negatively charged polysaccharide molecules, e.g., heparin sulfate. Significantly, the amino acid sequence of each of the four exon versions of huTSP-30a, b, c, and d (described below and shown in FIGS. 13-16) comprises an intact leader, N-terminal heparin binding cluster, both cysteine repeat domains, and TSR, but lacks the C-terminal heparin binding clusters. Thus, without being bound to a particular theory, it is likely that the 4 exon versions of each of these proteins binds less well to one or more negatively charged extracellular polysaccharides than its corresponding five exon huTSP-30.

TSP-30a

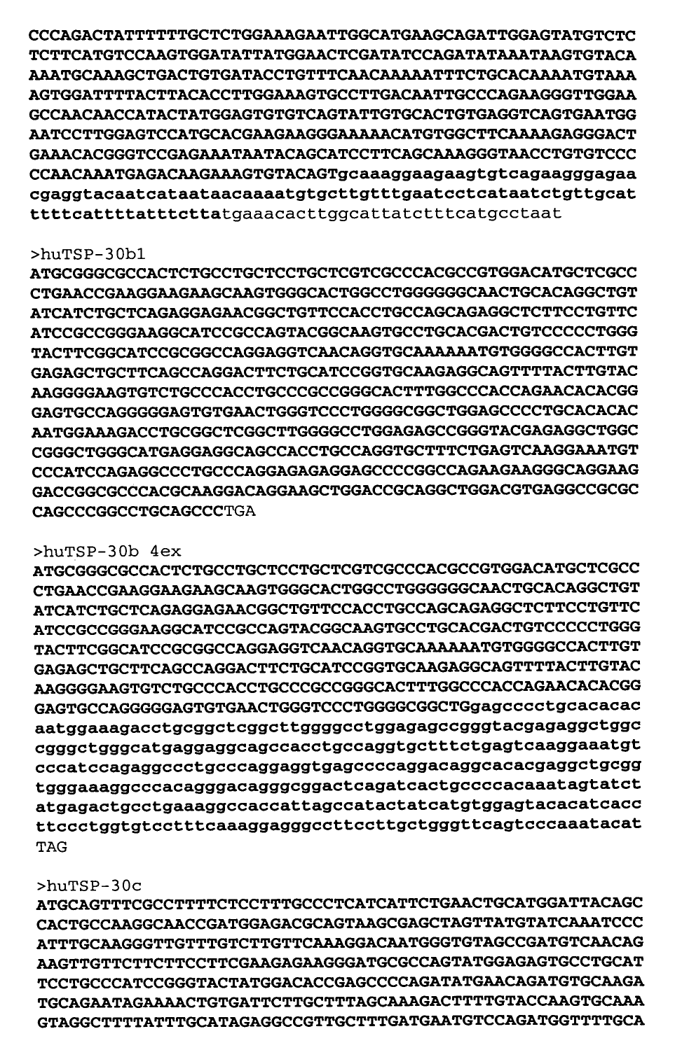

An alternative splicing pattern of the nucleic acid sequence of Celera DNA contig GA_x54KRFTFOF9, derived from Chromosome 6, produces the huTSP-30a 4ex sequence shown in FIG. 2, which encodes the huTSP-30a amino acid sequence shown in FIG. 1. FIG. 13 shows that the C-terminal portion of huTSP-30a 4 ex, comprising residues 213 through 235 as they are numbered in FIG. 13, is unique. Thus, in one embodiment, the polypeptides and polynucleotides of the present invention comprise an amino acid or nucleotide sequence, respectively, or a derivative, mutein, variant, fragment or fusion protein thereof, corresponding to all or some of huTSPa 4ex residues 213 through 235, or that lacks all or some of a sequence corresponding to huTSP-30a residues 213 through 272, as those sequences are numbered in FIG. 13, as described in more detail below.

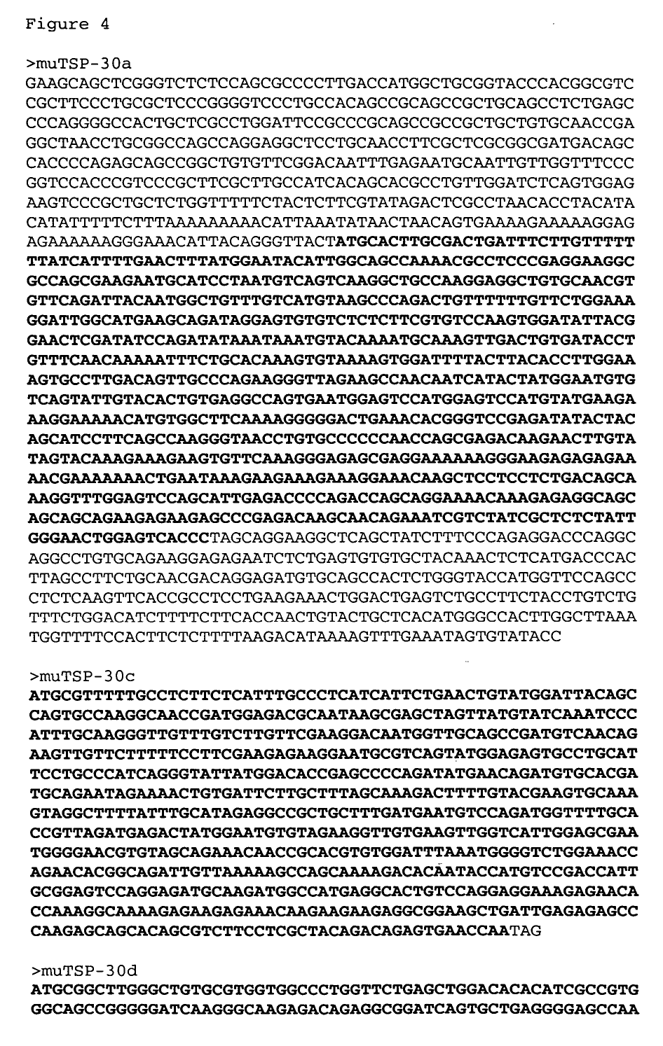

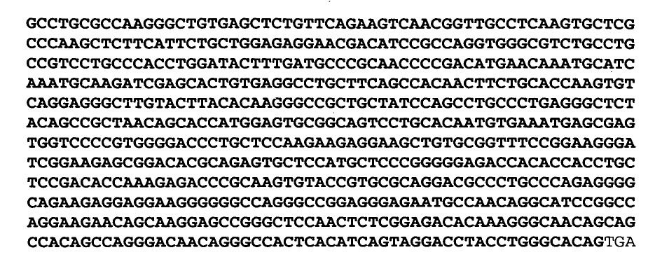

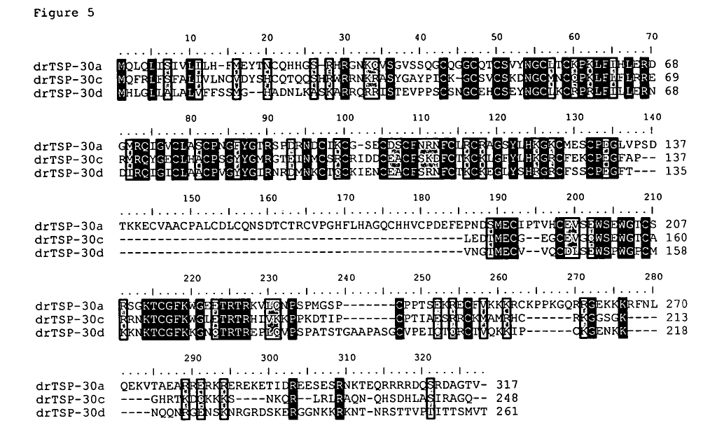

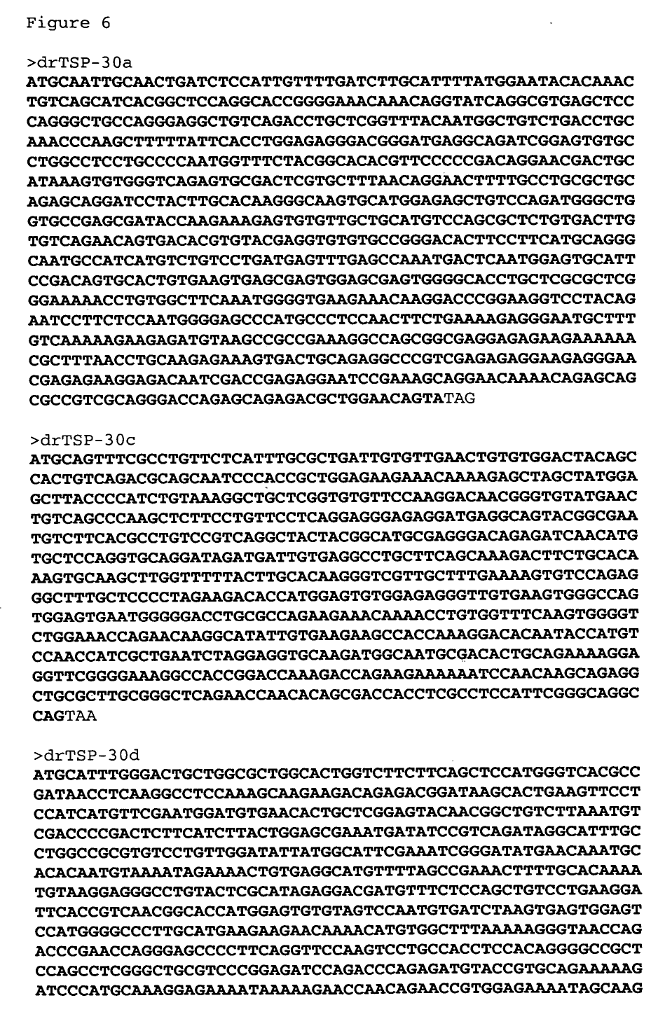

The muTSP-30a and drTSP-30a nucleotide sequences of FIGS. 4 and 6, respectively, which encode the polypeptide sequences of muTSP-30a (FIG. 3) and drTSP-30a (FIG. 5), respectively, were derived from cDNA molecules from the e8.5 mouse library (Invitrogen, Carlsbad, Calif.) and zebrafish 24 hour post-fertilization cDNA, respectively.

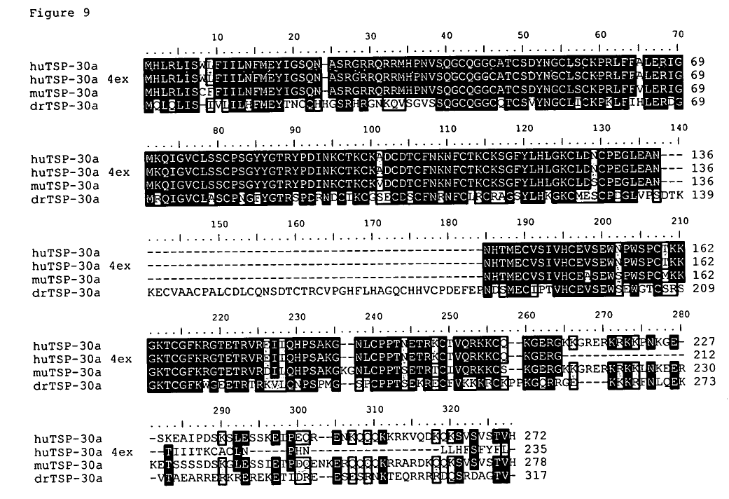

FIG. 9 shows that the TSP-30a proteins from humans, mice and zebrafish are highly conserved. The sequence similarity between these proteins is particularly high in the cysteine repeat domains, extending from about residue 39 to about residue 134, and in the TSR repeat, extending from about residue 194 to about residue 258, as the sequences are numbered in FIG. 9. The sequence of the zebrafish TSP-30a protein differs from its human and mouse counterparts in that it contains an approximately 45 amino acid insert relative to them. Interestingly, this inserted sequence lies between the second cysteine repeat domain and the TSR domain, and it appears to comprise two cysteine residues followed by another cysteine repeat domain. Thus, drTSP-30a appears to comprise a total of three cysteine repeat domains.

As shown in Example 2, drTSP-30a-derived transcripts were detected in the central nervous system (e.g., the brain), in discrete points along the midline, and in the dorsal artery of 24 hour old zebrafish embryos.

TSP-30b

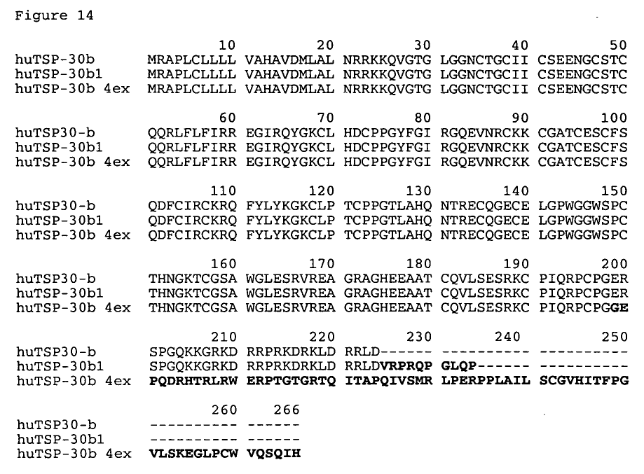

The sequence designated herein huTSP-30b was found to be a truncated version of huTSP-30b1, the full-length sequence. A cDNA was isolated from a human adult lung cDNA library and found to encode a four-exon splice variant. Accordingly, this variant has been designated huTSP-30b 4ex. The TSP-30b gene is on Chromosome 2 and is upstream and adjacent to ANG4. As shown in FIG. 14, huTSP-30b 4ex comprises a unique C-terminal sequence, extending from amino acid residues 199 through 266. The huTSP-30b1 sequence also comprises a unique C-terminal sequence from amino acid residues 225 to 234 as they are numbered in FIG. 14. Thus, in one aspect, the present invention provides polypeptides and polynucleotides comprising amino acid and nucleotide sequences, respectively, corresponding to some or all of the residues 199 through 266 of huTSP-30b 4×, residues 225 to 234 of huTSP-30b1, and/or lacking all or some of residues 199 through 224 of huTSP-30b, as the residues are numbered in FIG. 14, and fragments, derivatives, muteins, variants and fusion proteins thereof, as described in greater detail below.

As shown in Example 1, huTSP-30b-derived transcripts were detected in adult lung, testis, brain, spinal cord, and skin, and in fetal lung, skeletal muscle, brain, and colon. huTSP-30b-derived transcripts also were detected in ovarian endometrioid cancer cell line CRL 11731 (TOV 112D) the ovarian clear cell carcinoma CRL 11730 (TOV-21G), the ovarian adenocarcinoma cell line HTB-75 (CAOV-3), and the breast carcinoma cell line NCI-AND-RES. Low expression was also found in the melanoma cell line WM-9.

TSP-30c

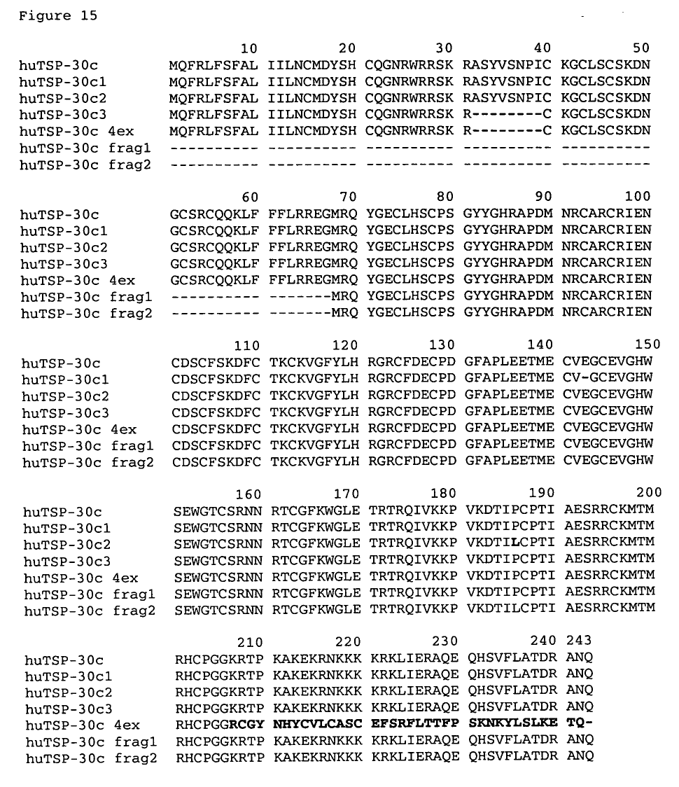

cDNA molecules comprising the huTSP-30c1, huTSP-30c2, huTSP-30c3 and huTSP-30c 4ex nucleotide sequences shown in FIG. 2, which encode, respectively, the huTSP-30c1, huTSP-30c2, huTSP-30c3 and huTSP-30c 4ex polypeptide sequences shown in FIG. 15, were isolated from a human adult lung cDNA library. The huTSP-30c1 differs from the huTSP-30c amino acid sequence in that it lacks the glutamate residue at position 143 as the residues are numbered in FIG. 15. The huTSP-30c2 amino acid sequence differs from the huTSP-30c sequence in that it has a leucine residue in place of a proline residue at position 186 as the residues are numbered in FIG. 15.

In one aspect, the present invention provides TSP-30c polypeptides and polynucleotides, and fragments, derivatives, muteins, variants and fusion proteins thereof, lacking the amino acid and nucleotide residues, respectively, corresponding to residue 143 of huTSP-30c, or lacking all or part of the nucleotide or amino acid sequence corresponding to residues 32 through 39 of huTSP-30c, or lacking all or part of the nucleotide or amino acid sequence corresponding to amino acid residues 207 to 243 of huTSP-30c, or having the nucleotide or amino acid residues corresponding to residue 186 of huTSP-30c2, or having all or part of the nucleotide or amino acid sequence corresponding to residues 207 to 209 of huTSP-30c 4ex, as the residues are numbered in FIG. 15.

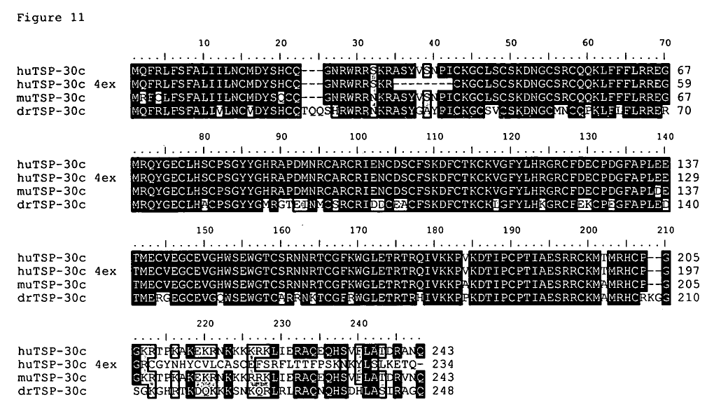

The muTSP-30c and drTSP-30c nucleotide sequences of FIGS. 4 and 6, respectively, which encode the polypeptide sequences of muTSP-30c (FIG. 3) and drTSP-30c (FIG. 5), respectively, were derived from cDNA molecules from the mouse e8.5 cDNA library (Invitrogen, Carlsbad, Calif.) and zebrafish 24 hours post-fertilization cDNA, respectively. As illustrated in FIG. 11, the level of conservation between human, mouse and zebrafish TSP-30c proteins is remarkable, particularly in the cysteine repeat domains (about residue 46 to about residue 83 and about residue 104 to about residue 137 as they are numbered in FIG. 11) and the TSR domain (about residue 148 to about residue 207 as they are numbered in FIG. 11).

As shown in Example 1, huTSP-30c-derived transcripts were detected in fetal and adult brain and lung, in the adult stomach, colon, small intestine and placenta, and in ovarian endometrioid cancer cell line CRL 11731 (TOV 112D). As shown in Example 2, drTSP-30c-derived transcripts were detected in the central nervous system (e.g., the brain), in discreet points along the midline, and in the apical region of the fin fold of 24 hour zebrafish embryos.

TSP-30d

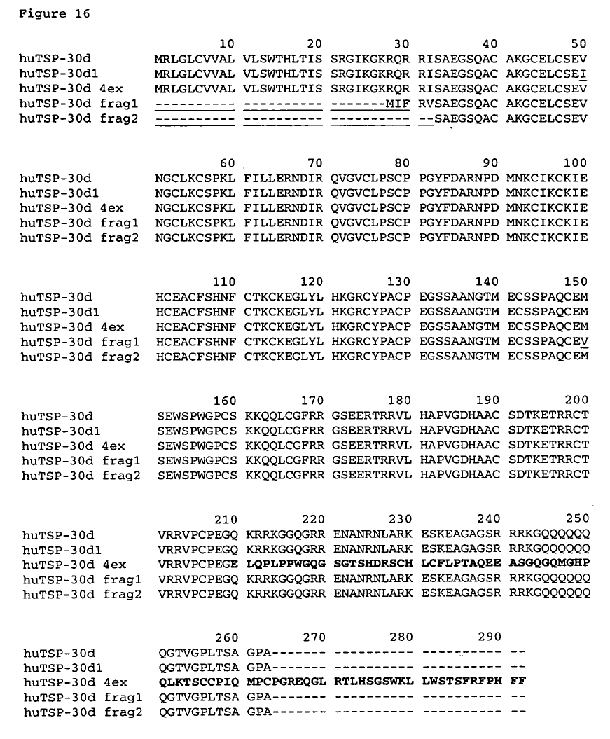

A polypeptide encoded by a four-exon splice variant of huTSP-30d, designated huTSP-30d 4ex, was predicted from Celera genomic contig GA.x5YUV32W802 and is provided in FIG. 1. A cDNA comprising the huTSP-30d nucleotide sequence shown in FIG. 2, which encodes the huTSP-30d polypeptide sequence shown in FIG. 1, was isolated from a human adult lung cDNA library. This sequence differs from related huTSP-30d sequences as shown in FIG. 16. Thus, in one aspect, the present invention provides polypeptides and polynucleotides comprising amino acid and nucleotide sequences, respectively, corresponding to all or part of residues 1 through 32 of huTSP-30d, residue 50 of huTSP-30d, and/or residue 150 of huTSP-30d, as the residues are numbered in FIG. 16, and fragments, derivatives, muteins, variants and fusion proteins thereof, as described in greater detail below.

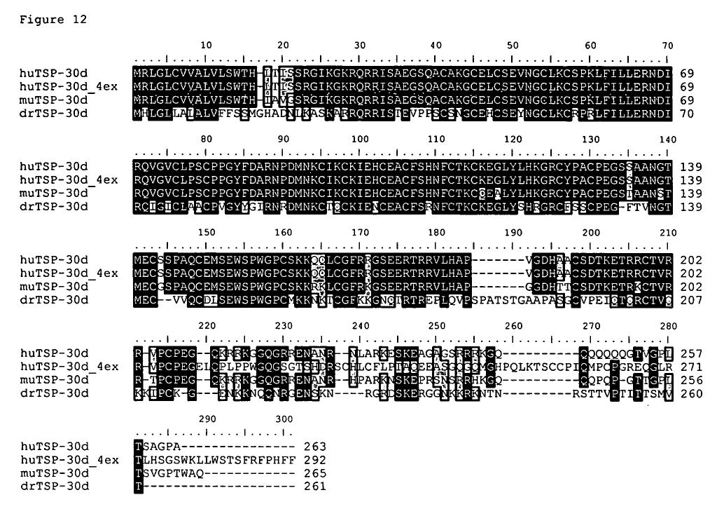

The muTSP-30d and drTSP-30d nucleotide sequences of FIGS. 4 and 6, respectively, which encode the polypeptide sequences of muTSP-30d (FIG. 3) and drTSP-30d (FIG. 5), respectively, were derived from the mouse e8.5 cDNA library (Invitrogen, Carlsbad, Calif.) and zebrafish 24 hour post-fertilization cDNA, respectively. Like the other TSP-30 proteins, TSP-30d is highly conserved between humans, mice and zebrafish (FIG. 12). The similarity between these homologs is greatest in the two cysteine repeats (about residue 44 to about residue 82 and about residue 102 to about residue 131 as they are numbered in FIG. 12). The TSR domain (about residue 149 to about residue 216 as they are numbered in FIG. 12) is more highly conserved in its N-terminal half than in its C-terminal half, primarily due to the presence in drTSP-30d of a short sequence of amino acids (residues 191 through 202 as they are numbered in FIG. 12) that is absent in the mouse and human homologs.

As shown in Example 1, huTSP-30d-derived transcripts were detected in adult human lung, digestive tract (including stomach, small intestine, and colon), prostate, testis, placenta and uterus, brain, spinal cord and skin, in human fetal lung, skeletal muscle, brain and colon, and in ovarian endometrioid cancer cell line CRL 11731 (TOV 112D),ovarian adenocarcinoma cell line HTB-161 (NIH OVCAR-3), and lung carcinoma cell line CCL-185 (A-549). In contrast to huTSP-30b-derived transcripts, huTSP-30d-derived transcripts were detected in mammary adenocarcinoma cell line HTB-22 (MCF-7), but not NCI/AND-RES.

As shown in Example 2, drTSPd-derived transcripts were detected in the central nervous system (e.g., the brain), in discreet points along the midline, and in the dorsal artery of 24 hour zebrafish embryos.

The sequence homology and expression data indicate that, like MK, the TSP-30a, b, c, and d proteins are growth factors that have diverse effects in various tissues and stages of development. For example, TSP-30 proteins likely play a role in one or more aspects of neural development (e.g., in neurite outgrowth, neuronal migration and targeting, formation of neural circuits, glial neuronal signaling, neurogenesis, and neural cell adhesion), tissue growth and/or repair (e.g., epithelial growth, wound healing, and tissue modeling), or other processes (e.g., stem cell growth and differentiation factor, vascular factor, endothelial differentiation, endothelial migration and targeting, immune cell migration and targeting, immune cell activation, stromal cell growth factor release, and inhibition of apoptosis).

Polypeptides of the Invention

In addition to the TSP-30 proteins described above, the present invention provides isolated fragments, muteins, variants, derivatives and fusion proteins thereof, non-limiting examples of which are provided below.

Fragments

In one aspect, the invention provides a polypeptide comprising one or more isolated fragments of a TSP-30 protein. A fragment of a protein is a polypeptide consisting of a sequence of amino acids that is identical to a subsequence of amino acids found in a larger (e.g., a wild-type or naturally-occurring protein) polypeptide sequence. The fragments can be of any size. The upper limit of the size of the fragment is determined only by the length of the full-length polypeptide sequence from which the fragment is derived. The lower limit of the size of the fragment is one amino acid. However, it is preferred that the fragment comprises a sequence that is unique to one of the sequences provided herein, examples of which are provided above. In one embodiment, the fragment is at least 5, 10, 15, 20, 25, 30, 35, 40, 45, 50, 55, 60, 65, 70, 75, 80, 85, 90, 95, 100, 125, 150, 175, 200, 225, or 250 amino acid residues in length. In another embodiment, the fragment is less than 250, 225, 200, 175, 150, 125, 100, 95, 90, 85, 80, 75, 70, 65, 60, 55, 50, 45, 40, 35, 30, 25, 20, 15, 10 or 5 amino acid residues in length. In another embodiment, the fragment comprises all but the N-terminal and/or C-terminal most 1, 2, 3, 4, 5, 6, 7, 8, 9, or 10 amino acids of an amino acid sequence illustrated in FIG. 1, 3, 5, 9, 10, 11, 12, 13, 14, 15 or 16. In another embodiment, the fragment comprises a sequence selected from the group consisting of the sequences indicated in bold in FIGS. 13-16. The fragment can include, for example, one or more domains from TSP-30a, b, c, or d, such as a domain identified above (e.g., a leader sequence domain, a cysteine repeat domain, a TSR domain, or a heparin binding cluster). In another embodiment, the fragment has a biological activity of a TSP-30a, b, c or d protein. In another embodiment, the fragment inhibits the biological activity of a TSP-30a, b, c or d molecule. Examples of biological activities associated with a TSP-30a, b, c, or d include being antigenic, binding to a particular antibody, binding to heparin sulfate, and modulating an angiogenic process, such as endothelial cell proliferation, migration or morphogenesis.

The discovery of the four exon versions of each of huTSP-30a, b, c, and d, which comprise intact leader sequences, N-terminal heparin binding clusters, two cysteine repeats, and TSRs, but differ from their corresponding five exon counterparts in that they lack C-terminal heparin binding clusters, suggests that huTSP-30 molecules lacking the C-terminal heparin binding clusters play an important physiological role. Without wishing to be bound to a particular theory, they could, for example, interact with a different set of molecules or diffuse farther from their point of origin than their corresponding five exon huTSP-30. Alternatively, the four exon forms could negatively regulate their five exon counterparts by, for example, competing for binding to molecules that bind to both the four and five exon forms of a huTSP-30. Thus, in one aspect, the present invention provides polypeptides that comprise one or more domains of a huTSP-30 selected from the group consisting of a leader sequence domain, an N-terminal heparin binding cluster domain, a cysteine repeat domain, and a TSR domain, wherein said polypeptide does not comprise a C-terminal heparin binding cluster. In another embodiment, the polypeptide comprises two, three, four, or more such domains, in any combination, e.g., two cysteine repeat domains, two TSR domains, a cysteine repeat domain and a TSR domain, two cysteine repeat domains and two TSR domains, etc. The domains can be from any TSP-30, for example, they can all be from the same TSP-30, or from different TSP-30 molecules, or homolgous TSP-30 molecules from different species. In another embodiment, the polypeptide further comprises a sequence from a four exon huTSP-30 4ex shown in bold in FIG. 13, 14, 15, or 16. In another embodiment, the polypeptide further comprises a heterologous sequence, examples of which are provided below. In another embodiment, the heterologous sequence promotes multimerization of the polypeptide, e.g., dimerization or trimerization. In another embodiment, the heterologous sequence is an Fc fragment or a leucine zipper. In another embodiment, the polypeptide comprises muteins, derivatives, fragments, or variants of the domains, examples of which are described below.

Muteins and Variants

In another aspect, the present invention provides isolated muteins or variants, which are polypeptides that differ from the TSP-30a, b, c or d polypeptides and fragments described herein by one or more amino acid additions, deletions, substitutions, or combinations thereof. In one embodiment, the mutein or variant comprises a sequence selected from the group consisting of residues 213 through 235 of huTSP-30a 4ex as shown in FIG. 13, residues 225 through 234 of huTSP-30b1 as shown in FIG. 14, residues 198 through 266 of huTSP-30b 4ex as shown in FIG. 14, residue 186 of huTSP-30c2 as shown in FIG. 15, residues 207 through 242 of huTSP-30c 4ex as shown in FIG. 15, or residues 210 through 233 of huTSP-30d 4ex as shown in FIG. 16. In another embodiment, the mutein or variant comprises a sequence that differs by 1, 2, 3, 4, 5, 6, 7, 8, 9, or 10 amino acids from a sequence selected from the group consisting of residues 213 through 235 of huTSP-30a 4ex as shown in FIG. 13, residues 225 through 234 of huTSP-30b1 as shown in FIG. 14, residues 198 through 266 of huTSP-30b 4ex as shown in FIG. 14, residue 186 of huTSP-30c2 as shown in FIG. 15, residues 207 through 242 of huTSP-30c 4ex as shown in FIG. 15, residues 210 through 233 of huTSP-30d 4ex as shown in FIG. 16, wherein each amino acid difference is, independently, an insertion, deletion, or substitution of an amino acid in the sequence. In another embodiment, the polypeptide comprises the sequence of huTSP-30d 4ex as shown in FIG. 16, except that it comprises amino acids or gaps corresponding to residues 1 through 30 of huTSP-30d frag1 as shown in FIG. 16, residues 1 through 32 of huTSP-30d frag2 as shown in FIG. 16, residue 50 of huTSP-30d1 as shown in FIG. 16, and residue 150 of huTSP-30d frag1 as shown in FIG. 16. In another embodiment, the mutein or variant comprising the sequence has a biological activity associated with a TSP-30a, b, c, and/or d protein, e.g., binding an anti-TSP-30a, b, c, and/or d antibody.

In another embodiment, a polypeptide of the invention comprises a TSP-30a sequence that lacks all or some of residues 213 through 272 of huTSP-30a as shown in FIG. 13. In another embodiment, the polypeptide further comprises a sequence of at least 10, 15, 20, 25, 30, 35, 40, 45, 50, 60, 70, 80, 90, 100, 125, 150, 175, 200, or 210 contiguous amino acid residues of huTSP-30a as shown in FIG. 13. In another embodiment, a polypeptide of the invention comprises a TSP-30b sequence that lacks all or some of residues 199 through 224 of huTSP-30-b as shown in FIG. 14. In another embodiment, the polypeptide further comprises a sequence of at least 10, 15, 20, 25, 30, 35, 40, 45, 50, 60, 70, 80, 90, 100, 125, 150, 175, or 190 contiguous amino acid residues of huTSP-30b as shown in FIG. 14. In another embodiment, a polypeptide of the invention comprises a TSP-30c sequence that lacks all or some of residues 32 through 39 of huTSP-30c as shown in FIG. 15. In another embodiment, the polypeptide further comprises a sequence of at least 10, 15, 20, 25, 30, 35, 40, 45, 50, 60, 70, 80, 90, 100, 125, 150, 175, or 200 contiguous amino acid residues of huTSP-30c as shown in FIG. 15. In another embodiment, a polypeptide of the invention comprises a TSP-30c sequence that lacks residue 143 of huTSP-30c as shown in FIG. 15. In another embodiment, the polypeptide further comprises a sequence of at least 10, 15, 20, 25, 30, 35, 40, 45, 50, 60, 70, 80, 90, 100, 125 or 140 contiguous amino acid residues of huTSP-30c as shown in FIG. 15. In another embodiment, a polypeptide of the invention comprises a TSP-30c sequence that lacks residue 184 of huTSP-30c as shown in FIG. 15. In another embodiment, the polypeptide further comprises a sequence of at least 10, 15, 20, 25, 30, 35, 40, 45, 50, 60, 70, 80, 90, 100, 125, 150, 175 or 185 contiguous amino acid residues of huTSP-30c as shown in FIG. 15. In another embodiment, a polypeptide of the invention comprises a TSP-30c sequence that lacks all or some of residues 207 through 243 of huTSP-30c as shown in FIG. 15. In another embodiment, the polypeptide further comprises a sequence of at least 10, 15, 20, 25, 30, 35, 40, 45, 50, 60, 70, 80, 90, 100, 125, 150, 175 or 200 contiguous amino acid residues of huTSP-30c as shown in FIG. 15. In another embodiment, the invention comprises a TSP-30d sequence that lacks all or some of residues 210 through residues 263 of huTSP-30d as shown in FIG. 16. In another embodiment, the polypeptide further comprises a sequence of at least 10, 15, 20, 25, 30, 35, 40, 45, 50, 60, 70, 80, 90, 100, 125, 150, 175 or 200 contiguous amino acid residues of huTSP-30d as shown in FIG. 15.

In another embodiment, the mutein or variant has a biological activity of a TSP-30a, b, c or d protein. In another embodiment, the mutein or variant inhibits the biological activity of a TSP-30a, b, c, or d molecule. Examples of biological activities associated with a TSP-30a, b, c, or d are described below, and include being antigenic, binding to heparin sulfate, and modulating an angiogenic process, such as endothelial cell proliferation, migration or morphogenesis.

As described above, TSP-30 variants were found to be encoded by cDNA molecules comprising 4 exons (the “4ex” forms of the TSP-30 molecules). Thus, the present invention provides for each TSP-30 5ex molecule a corresponding 4ex variant, e.g., huTSP-30a 4ex, muTSP-30a, muTSP-30a 4ex, drTSP-30a/b, drTSP-30a 4ex, huTSP-30b, huTSP-30b 4ex, muTSP-30b, muTSP-30b 4ex, huTSP-30c1, huTSP-30c2, huTSP-30c3, huTSP-30c 4ex, huTSP-30c1 4ex, huTSP-30c2 4ex, huTSP-30c3 4ex, muTSP-30c, muTSP-30c 4ex, drTSP-30c, drTSP-30c 4ex, huTSP-30d, huTSP-30d 4ex, muTSP-30d, muTSP-30d 4ex, drTSP-30d, and drTSP-30d 4ex.

Several lines of evidence indicate that the sequence domains of the TSP-30 proteins (e.g., the leader sequence, heparing binding clusters, cysteine repeats, and TSRs) are functionally independent domains. First, as described above, the TSP-30 proteins have distinct sequence domains. Second, as described above, the domain organization of the TSP-30 proteins is very similar to that of MK. The N- and C-terminal sequence domains of MK correspond to functional domains that can be independently mutated or isolated. Third, drTSP-30a is unlike the other TSP30 molecules described herein in that it comprises a third cysteine repeat domain, which is located after the second cysteine repeat domain and before the TSR domain. Fourth, each of the huTSP-30 4ex polypeptides described herein comprises intact cysteine repeat and TSR domains, and diverges from the sequence of the five-exon huTSP-30 polypeptides in the short region between the TSR and heparin binding cluster domains. Thus, it is likely that the sequence domains of the TSP-30 proteins are functionally independent. Accordingly, in one aspect, the present invention provides muteins, variants, fragments, derivatives, conjugates and fusion proteins comprising one or more sequence domains of a TSP-30 polypeptide. The sequence domains can be, for example, isolated, rearranged, fused to heterologous peptides, or otherwise manipulated, without destroying its biological function. In another embodiment, the present invention provides a polypeptide comprising a TSP-30 sequence comprising a sequence domain that is mutated, deleted, or otherwise modified, without disturbing the biological function of another sequence domain in the polypeptide. Such a mutant, variant, fragment, conjugate, fusion protein or derivative could be used to reduce the biological activity of a wild-type TSP-30 protein, for example, by competing with a wild-type TSP-30 for a substrate or activator. Conversely, such a mutant, variant, fragment, conjugate, fusion protein or derivative could be used to increase the biological activity of a wild-type TSP-30 protein, for example, by having a biological activity that is insensitive to an inhibitor that affects the wild-type TSP-30 protein, or by competing with the wild-type TSP-30 for binding to an inhibitor.

The high degree of similarity between TSP-30 homologs in humans, mice and zebrafish indicates that these molecules are highly conserved across distantly related animal species. Accordingly, in another aspect, the present invention provides TSP-30a, b, c and d molecules from mammals, for example, primates (e.g., humans, monkeys and apes), rodents (e.g., mice, rats and hamsters), canines, felines, bovines, ovines and equines, fish (e.g., zebrafish, salmon, trout, and sharks), birds, reptiles and amphibians. In one embodiment, a TSP-30 polypeptide derived from a non-human species is used to treat an illness, injury, disease or condition in a human subject. A high degree of sequence similarity between homologous proteins from distantly related species typically indicates that the proteins perform similar and important roles in vivo. Accordingly, in one aspect, the invention provides methods of studying the function of a TSP-30 protein from a first species by studying the function of it, or a homolog of it, in a second species. In one embodiment, the first species is a human and the second species is a non-human species, for example, a mammal (e.g., a non-human primate or rodent), a bird, a reptile, an amphibian, or a fish. In another embodiment, the TSP-30 protein is a human TSP-30 protein. In another embodiment, the homolog of the TSP-30 protein is a non-human TSP-30 protein, for example, a TSP-30 protein from a mammal (e.g., a non-human primate or rodent), a bird, a reptile, an amphibian, or a fish.

Changes to the amino acid sequence (e.g., substitutions, additions or deletions) of a native polypeptide can be made conservatively. Examples of a conservative change include a change outside of a recognized sequence domain (e.g., a leader sequence, heparin binding cluster, cysteine repeat, or TSR domain), a change of an amino acid that is not strongly conserved within a sequence domain, a change of an amino acid that is not strongly conserved between homologous TSP-30 molecules from humans and mice, humans and zebrafish, mice and zebrafish, or humans, mice and zebrafish (e.g., between huTSP-30a 4ex and muTSP-30a 4ex), a change of a residue that is not strongly conserved between TSP-30a, TSP-30b, TSP-30c, and/or TSP-30d within a species (e.g., between huTSP-30a 4ex and huTSP-30b 4ex), a change that does not alter a biological activity of the native polypeptide, a change that does not alter an epitope of the native polypeptide, or a change that does not alter the secondary and/or tertiary structure of the native polypeptide. Additional examples include substituting one aliphatic residue for another, such as Ile, Val, Leu, or Ala for one another, or substitutions of one polar or charged residue for another, such as between Lys and Arg; Glu and Asp; or Gln and Asn, or substitutions of one aromatic residue for another, such as Phe, Trp, or Tyr for one another. Other such conservative substitutions, for example, substitutions of entire regions having similar hydrophobicity characteristics, are known in the art.

In various embodiments, the amino acid sequence of the TSP-30 variant is at least 70, 75, 80, 85, 90, 91, 92, 93, 94 95, 96, 97, 98, 99, or 99.25% identical to the amino acid sequence of a native TSP-30a, b, c, or d. Percent identity, in the case of both polypeptides and nucleic acids, can be determined by visual inspection. Percent identity also can be determined using the alignment method of Needleman and Wunsch, 1970, J. Mol. Biol. 48:443, as revised by Smith and Waterman, 1981, Adv. Appl. Math 2:482. Preferably, percent identity is determined by using a computer program, for example, the GAP computer program version 10.x available from the Genetics Computer Group (GCG; Madison, Wis., see also Devereux et al., 1984, Nucl. Acids Res. 12:387). The preferred default parameters for the GAP program include: (1) a unary comparison matrix (containing a value of 1 for identities and 0 for non-identities) for nucleotides, and the weighted comparison matrix of Gribskov and Burgess, 1986, Nucl. Acids Res. 14:6745, as described by Schwartz and Dayhoff, eds., Atlas of Protein Sequence and Structure, National Biomedical Research Foundation, pp.353-58, 1979 for amino acids; (2) a penalty of 30 amino acids or 50 nucleotides for each gap and an additional 1 amino acid or 3 nucleotide penalty for each symbol in each gap; (3) no penalty for end gaps; and (4) no maximum penalty for long gaps. Other programs used by one skilled in the art of sequence comparison may also be used. For fragments of TSP, the percent identity is calculated based on that portion of TSP that is present in the fragment.

In one embodiment, D-amino acids are substituted for the naturally occurring L-amino acids. D-amino acids provide improved stability under in vivo conditions. In addition, due to the size of the extracellular domain or soluble polypeptide sequence of the invention it may be advantageous to synthesize the polypeptide using D-amino acids. It will be recognized that the polypeptide of the invention can be synthesized such that the polypeptide comprises a combination of L- and D-amino acids.

Fusion Polypeptides

In another aspect, a polypeptide of the invention further comprises one or more additional amino acids and/or amino acid sequences. The one or more additional amino acids and/or amino acid sequences can be joined to the TSP-30 molecule using any technique known in the art. For example, they can be joined non-covalently (for example, via one or more salt bridges, hydrogen bonds, and/or hydrophobic interactions) or covalently. In one embodiment, the various amino acids and peptides are joined to each other via peptide bonds, such that they form a linear and sequence of contiguously joined amino acids having an amino terminus and a carboxy terminus. In another embodiment, the fusion protein comprises amino acids joined to each other via peptide binds such that they form a circular polypeptide, that is, one not having an N-terminus or a C-terminus, but that can be cleaved between any two contiguous amino acid residues to yield a linear polypeptide having an N-terminus and a C-terminus. In another embodiment, the polypeptides are joined to each other indirectly, through another molecule, for example, a molecule that is bound by both polypeptides (e.g., a polysaccharide).

Fusion proteins of the invention comprise one or more sequences derived from TSP-30a, b, c, and/or d and one or more amino acids or amino acid sequences that are heterologous to TSP-30a, b, c or d. The sequences derived from TSP-30a, b, c, and/or d can be, for example, can comprise the full-length TSP-30a, b, c, and/or d amino acid sequences, or one or more fragments, derivatives, variants, muteins and conjugates thereof, examples of which are described herein. These sequences can have one or more attributes of an isolated TSP-30a, b, c, and/or d molecule, for example, a sequence domain, (e.g., a heparin binding cluster, a cysteine repeat, a TSR domain, or a leader sequence), a function, (e.g., modulating one or more angiogenic processes, binding an anti-TSP-30a, b, c, or d antibody, or binding a molecule (e.g., an activator, inhibitor, or receptor) that binds to TSP-30a, b, c, or d. The heterologous amino acids or peptides can comprise any amino acid sequence. Typically, a heterologous peptide will impart to the fusion protein a new or improved property or characteristic, for example, ease of isolation, increased stability, reduced or increased antigenicity, or increased activity. Examples of heterologous peptides include Fc fragments, leucine zippers, and peptide linkers, which are described in more detail below. In one embodiment, the invention provides compositions and fusion proteins that comprise at least one TSR domain.

In another aspect, the present invention provides polypeptides that are soluble. Soluble polypeptides are capable of being secreted from the cells that express them. The use of soluble forms of polypeptides is advantageous for certain applications. Purification of the polypeptides from recombinant host cells is facilitated since the polypeptides are secreted, and soluble proteins are generally suited for parenteral administration. A secreted soluble polypeptide may be identified (and distinguished from its non-soluble membrane-bound counterparts) by separating intact cells which express the desired polypeptide from the culture medium, e.g., by centrifugation, and assaying the medium (supernatant fraction) for the presence of the desired polypeptide. The presence of the desired polypeptide in the medium indicates that the polypeptide was secreted from the cells and thus is a soluble form of the polypeptide. Soluble polypeptides may be prepared by any of a number of conventional techniques. A polynucleotide encoding a desired soluble polypeptide may be subcloned into an expression vector for production of the polypeptide, or the desired encoding polynucleotide or soluble polypeptide may be chemically synthesized, using techniques that are well-known in the art.

In one embodiment, soluble TSP-30 polypeptides of the invention comprise all or part of TSP-30a, b, c and/or d, or a derivative, variant, mutein, fusion protein, or conjugate thereof. Soluble TSP-30 polypeptides advantageously comprise a native or heterologous signal peptide when initially synthesized, to promote secretion from the cell, but the signal sequence can be cleaved during or after secretion. The ability of these related forms to modulate angiogenesis or other TSP-30a, b, c or d-mediated responses may be determined in vitro or in vivo, using methods such as those exemplified below or using other assays known in the art.

In another aspect of the present invention a multimeric form of a TSP-30a, b, c, and/or d polypeptide is provided. TSP-30 multimers are covalently-linked or non-covalently-linked multimers, including dimers, trimers, or higher multimers. Multimers may be linked by disulfide bonds formed between cysteine residues on different soluble TSP-30 polypeptides. One embodiment of the invention is directed to multimers comprising multiple TSP-30 polypeptides joined via covalent or non-covalent interactions between peptide moieties fused to the TSP-30 polypeptides. In one embodiment peptide linkers are fused to the C-terminal end of a first soluble TSP-30 molecule and the N-terminal end of a second soluble TSP-30 molecule. This structure may be repeated multiple times such that at least one, preferably 2, 3, 4, or more soluble TSP-30 polypeptides are linked to one another via peptide linkers at their respective attached thereto, as described in more detail below. For example, a polypeptide of the invention comprises a sequence Z₁-X-Z₂, wherein Z₁ and Z₂ are each individually, for example, a polypeptide comprising a cysteine repeat domain, a TSR domain, or a heparin binding cluster. In particular embodiments, the multimers comprise Z₁-X-Z₂(-X-Z)ₙ, wherein ‘n’ is any integer, but is preferably 1 or 2. In a further embodiment, the peptide linkers should be of sufficient length to allow a TSP polypeptide to form bonds with an adjacent TSP polypeptide. Examples of peptide linkers include (using the single-letter code for amino acids) GGGGS (SEQ ID NO:50), (GGGGS)ₙ (SEQ ID NO:50), GKSSGSGSESKS (SEQ ID NO:51), GSTSGSGKSSEGKG (SEQ ID NO:52), GSTSGSGKSSEGSGSTKG (SEQ ID NO:53), GSTSGSGKPGSGEGSTKG (SEQ ID NO:54), or EGKSSGSGSESKEF (SEQ ID NO:55). Linking moieties are described, for example, in Huston et al., 1988, PNAS 85:5879-83, Whitlow et al., 1993, Protein Engineering 6:989-95, and Newton et al., 1996, Biochemistry 35:545-53. Other suitable peptide linkers are those described in U.S. Pat. Nos. 4,751,180 and 4,935,233, which are hereby incorporated by reference. A polynucleotide encoding a desired peptide linker can be inserted between, and in the same reading frame as, a polynucleotide encoding a TSP-30 polypeptide, using any suitable conventional technique. In particular embodiments, a fusion polypeptide comprises from two to four TSP-30 polypeptides separated by peptide linkers.

In another aspect, the present invention provides a polypeptide that comprises a peptide that has the property of promoting oligomerization. An “oligomerizing peptide” is a peptide that stably interacts with identical or similar polypeptides under certain conditions, for example, within a cell, or in the extracellular milieu, thus forming an oligomer. Leucine zippers and certain polypeptides derived from antibodies are among the peptides that can promote oligomerization.

In some embodiments, a TSP-30 oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, Proc. Natl. Acad. Sci. USA 88:10535; Byrn et al., 1990, Nature 344:677; and Hollenbaugh et al., “Construction of Immunoglobulin Fusion Proteins,” in Current Protocols in Immunology, Suppl. 4, pages 10.19.1-10.19.11, 1992).

One preferred embodiment of the present invention is directed to a TSP-30-Fc dimer comprising two fusion proteins created by fusing a TSP-30 polypeptide to an Fc polypeptide. A gene fusion encoding the TSP-30-Fc fusion protein is inserted into an appropriate expression vector. TSP-30-Fc fusion proteins are expressed in host cells transformed with the recombinant expression vector, and allowed to assemble much like antibody molecules, whereupon interchain disulfide bonds form between the Fc moieties to yield a divalent (oligomeric) TSP-30 polypeptide. The term “Fc polypeptide” as used herein includes native, variant and mutein forms of polypeptides derived from the Fc region of an antibody. Truncated forms of such polypeptides containing the hinge region that promotes oligomerization are also included.

One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and by Baum et al., 1994, EMBO J. 13:3992. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. Fusion polypeptides comprising Fc moieties, and oligomers formed therefrom, offer an advantage of facile purification by affinity chromatography over Protein A or Protein G columns, and Fc fusion polypeptides may provide a longer in vivo half life, which is useful in therapeutic applications, than unmodified polypeptides.

In other embodiments, a TSP-30 polypeptide may be substituted for the variable portion of an antibody heavy or light chain. If fusion proteins are made with both heavy and light chains of an antibody, it is possible to form a TSP-30 oligomer with as many as four TSP-30 polypeptides.

Another method for preparing TSP-30 oligomers involves use of a leucine zipper domain. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that oligomerize (e.g., dimerize or trimerize). Examples of leucine zipper domains suitable for producing soluble oligomerized proteins are described in PCT application WO 94/10308, and the leucine zipper derived from lung surfactant protein D (SPD) described in Hoppe et al., 1994, FEBS Lett. 344:191. The use of a modified leucine zipper that allows for stable trimerization of a heterologous protein fused thereto is described in Fanslow et al., 1994, Semin. Immunol. 6:267. Recombinant fusion proteins comprising a TSP-30 polypeptide fused to a leucine zipper peptide can be expressed in suitable host cells, and the TSP-30 oligomers that form can be recovered from the culture supernatant.

For some applications, the TSP-30 oligomers of the present invention are believed to provide certain advantages. Fc fusion polypeptides, for example, typically exhibit an increased in vivo half-life as compared to an unmodified polypeptide.

The present invention encompasses the use of various forms of TSP-30 multimers and oligomers that retain a biological function (e.g., the ability to modulate angiogenesis). The term “TSP-30 multimer” is intended to encompass multimers containing all or part of one or more of the native TSP-30a, b, c, and/or d polypeptides as herein described, as well as related forms including, but not limited to, multimers of fragments, variants, muteins, derivatives, conjugates and fusion polypeptides of TSP-30. The ability of these related forms to modulate angiogenesis or other TSP-30a, b, c or d-mediated responses may be determined in vitro or in vivo, using methods such as those exemplified in the examples or using other assays known in the art.

Among the TSP-30 polypeptides, oligomers, and multimers useful in practicing the present invention are TSP-30 variants that has the ability to modulate angiogenesis. Such TSP-30 variants include polypeptides that are substantially similar to native TSP-30a, b, c, or d, but which have an amino acid sequence different from that of the native sequence because of one or more deletions, insertions or substitutions. Particular embodiments include, but are not limited to, TSP-30 polypeptides that comprise from one to ten amino acid deletions, insertions or substitutions compared to a native TSP-30 sequence. Included as variants of TSP-30 polypeptides are those variants that are naturally occurring, such as allelic forms and alternatively spliced forms, as well as variants that have been constructed by modifying the amino acid sequence of a TSP-30 polypeptide or the nucleotide sequence of a nucleic acid encoding a TSP-30 polypeptide.

The present invention further encompasses TSP-30 polypeptides with or without associated native-pattern glycosylation. TSP-30 polypeptides expressed in yeast or mammalian expression systems (e.g., COS-1 or COS-7 cells) may be similar to or significantly different from a native TSP-30 polypeptide in molecular weight and glycosylation pattern, depending upon the choice of expression system. Expression of TSP-30 polypeptides in bacterial expression systems, such as E. coli, provides non-glycosylated molecules. Different host cells may also process polypeptides differentially, resulting in heterogeneous mixtures of polypeptides with variable N- or C-termini.

The primary amino acid structure of TSP-30 polypeptides may be modified to create derivatives by forming covalent or aggregative conjugates with other chemical moieties, such as glycosyl groups, lipids, phosphate, acetyl groups and the like. Covalent derivatives of TSP-30 polypeptides may be prepared by linking particular functional groups to TSP-30 amino acid side chains or at the N-terminus or C-terminus of a TSP-30 polypeptide. In addition, TSP can be complexed with polyethylene glycol (PEG), metal ions, or incorporated into polymeric compounds such as polyacetic acid, polyglycolic acid, hydrogels, dextran, and the like, or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance, and are thus chosen according to the intended application.

Fusion polypeptides of TSP-30 polypeptides that are useful in practicing the invention also include covalent or aggregative conjugates of a TSP-30 polypeptide with other polypeptides added to provide novel polyfunctional entities.

TSP-30 Antibodies

In another aspect, the present invention provides antibodies and antibody derivatives that bind to a TSP-30a, b, c and/or d polypeptide as herein described. In one embodiment, the antibody binds to a polypeptide selected from the group consisting of huTSP-30a 4ex, muTSP-30a, muTSP-30a 4ex, drTSP-30a, drTSP-30a 4ex, huTSP-30b, huTSP-30b 4ex, huTSP-30c1, huTSP-30c2, huTSP-30c3, huTSP-30c 4ex, muTSP-30c, muTSP-30c 4ex, drTSP-30c, drTSP-30c 4ex, huTSP-30d, huTSP-30d 4ex, muTSP-30d, muTSP-30d 4ex, drTSP-30d, and drTSP-30d 4ex. In another embodiment, the antibody binds to a 4ex version of a TSP-30 polypeptide, but not to a 5ex version of the TSP-30 polypeptide. Such epitopes are useful for raising antibodies, and in particular the blocking monoclonal antibodies described in more detail below. Such epitopes or variants thereof can be produced using techniques well known in the art such as solid-phase synthesis, chemical or enzymatic cleavage of a polypeptide, or using recombinant DNA technology.

The claimed invention encompasses compositions and uses of antibodies that are immunoreactive with TSP-30 polypeptides. Such antibodies “bind specifically” to TSP-30 polypeptides, meaning that they bind via antigen-binding sites of the antibody as compared to non-specific binding interactions. The terms “antibody” and “antibodies” are used herein in their broadest sense, and include, without limitation, intact monoclonal and polyclonal antibodies as well as fragments such as Fv, Fab, and F(ab′)₂ fragments, single-chain antibodies such as scFv, and various chain combinations. The antibodies of the present invention can be, for example, human, humanized, or chimeric. The antibodies may be prepared using a variety of well-known methods including, without limitation, immunization of animals having native or transgenic immune repertoires, phage display, hybridoma and recombinant cell culture, and transgenic plant and animal bioreactors.

Both polyclonal and monoclonal antibodies may be prepared by conventional techniques. See, for example, Kennet et al. (eds.), Monoclonal Antibodies, Hybridomas: A New Dimension in Biological Analyses, Plenum Press, New York (1980); and Harlow and Land (eds.), Antibodies: A Laboratory Manual, Cold Spring Harbor Laboratory Press, Cold Spring Harbor, N.Y., (1988).

Hybridoma cell lines that produce monoclonal antibodies specific for the polypeptides of the invention are also contemplated herein. Such hybridomas may be produced and identified by conventional techniques. One method for producing such a hybridoma cell line comprises immunizing an animal with a polypeptide, harvesting spleen cells from the immunized animal, fusing said spleen cells to a myeloma cell line, thereby generating hybridoma cells, and identifying a hybridoma cell line that produces a monoclonal antibody that binds the polypeptide. The monoclonal antibodies produced by hybridomas may be recovered by conventional techniques.

The monoclonal antibodies of the present invention include chimeric antibodies, e.g., “humanized” versions of antibodies originally produced in mice or other non-human species. A humanized antibody is an engineered antibody that typically comprises the variable region of a non-human (e.g., murine) antibody, or at least complementarity determining regions (CDRS) thereof, and the remaining immunoglobulin portions derived from a human antibody. Procedures for the production of chimeric and further engineered monoclonal antibodies include those described in Riechmann et al., 1988, Nature 332:323, Liu et al., 1987, PNAS 84:3439, Larrick et al., 1989, Bio/Technology 7:934, and Winter and Harris, TIPS 14:139, May, 1993. Such humanized antibodies may be prepared by known techniques and offer the advantage of reduced immunogenicity when the antibodies are administered to humans.

Procedures that have been developed for generating human antibodies in non-human animals may be employed in producing antibodies of the present invention. The antibodies may be partially human or preferably completely human. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some, and preferably virtually all, antibodies produced by the animal upon immunization.

Mice in which one or more endogenous immunoglobulin genes have been inactivated by various means have been prepared and are commercially available from, for example, Medarex Inc. (Princeton, N.J.) and Abgenix Inc. (Fremont, Calif.). Human immunoglobulin genes have been introduced into the mice to replace the inactivated mouse genes. Antibodies produced in the animals incorporate human immunoglobulin polypeptide chains encoded by the human genetic material introduced into the animal. Examples of techniques for the production and use of such transgenic animals to make antibodies (which are sometimes called “transgenic antibodies”) are described in U.S. Pat. Nos. 5,814,318, 5,569,825, and 5,545,806, which are incorporated by reference herein.

Inhibitory Antisense, Ribozyme, and Triple Helix Approaches

Angiogenesis, a process associated with angiogenesis, or another activity associated with a TSP-30 protein can be modulated in a cell, group of cells, tissue, organ or subject by reducing the level of TSP-30a, b, c, and/or d activity using well-known antisense, gene “knock-out,” ribozyme and/or triple helix methods. Among the compounds that may exhibit the ability to reduce the activity, expression or synthesis of TSP-30a, b, c, and/or d, and thus modulate angiogenesis, are antisense, ribozyme, and triple helix molecules. Such molecules may be designed to reduce or inhibit either unimpaired, or if appropriate, mutant target gene activity. Techniques for the production and use of such molecules are well known to those of skill in the art.

TSP-30 Nucleic Acids

In another aspect, the present invention provides a polynucleotide encoding all or part of a TSP-30a, b, c, and/or d polypeptide, or a fragment, derivative, mutein, variant, conjugate or fusion protein thereof, examples of which are described above. In one embodiment, the TSP-30 polynucleotide comprises at least 10, 20, 30, 40, 50, 60, 70, 80, 90, 100, 125, 150, 175, 200, 250, 300, 350, 400, 450, 500, 600, 700, 800, 900, or 1000 contiguous nucleotide residues from a nucleotide sequence selected from those depicted in FIGS. 2, 4, and 6. In another embodiment, the TSP polynucleotide comprises a sequence that encodes an amino acid sequence selected from the group of amino acid sequences that are indicated in bold in FIGS. 13, 14, 15, and 16. In another embodiment, the fragment of a nucleic acid encoding TSP-30a, b, c, or d is at least 10, 15, 20, 25, 30, 35, 40, 45, 50, 55, 60, 65, 70, 75, 80, 85, 90, 95, 100, 125, 150, 175, 200, 250, 300, 350, 400, 450, 500, 750, or 1,000 nucleotides in length. In another embodiment, the PST-30 polynucleotide comprises a sequence that is at least 70, 75, 80, 85, 90, 95, 97, 98, 99, or 99.9% identical to a sequence consisting of at least 10, 20, 30, 40, 50, 60, 70, 80, 90, 100, 125, 150, 175, 200, 250, 300, 350, 400, 450, 500, 600, 700, 800, 900, or 1000 contiguous nucleotide residues from a nucleotide sequence selected from those depicted in FIGS. 2, 4, and 6. In another embodiment, the TSP polynucleotide fragment, derivative, mutein, variant or conjugate encodes a polypeptide that shares a property in common with a TSP-30a, b, c, and/or d polypeptide comprising a sequence selected from the group consisting of those depicted in FIGS. 2, 4, and 6. Examples of such properties include modulating angiogenesis, or one or more processes related to angiogenesis, such as endothelial cell proliferation, migration and morphogenesis, specifically binding a receptor or other protein, and binding a TSP-30a, b, c and/or d-specific antibody. In another embodiment, the TSP polynucleotide fragment, derivative, mutein, variant or conjugate reduces the biological activity of a TSP-30a, b, c, and/or d polypeptide, for example, by inhibiting the transcription, post-transcriptional processing, or translation of TSP-30 encoding nucleic acid. Examples of such TSP-30 polynucleotide fragments, derivatives, muteins, variants and conjugates include triple-helix forming nucleic acids, anti-sense nucleic acids, and inhibitory RNA (“iRNA”). In another embodiment, the TSP-30 polynucleotide fragment, derivative, mutein, variant or conjugate encodes a TSP-30 polypeptide that inhibits an activity of a polypeptide comprising an amino acid sequence selected from the group consisting of those depicted in FIGS. 1, 3, 5, 9, 10, 11, 12, 13, 14, 15, and 16. Examples of such activities include modulating angiogenesis, or one or more processes related to angiogenesis, such as endothelial cell proliferation, migration and morphogenesis, specifically binding a receptor or other protein, and binding a TSP-30a, b, c and/or d-specific antibody.

Due to degeneracy of the genetic code, there can be considerable variation in nucleotide sequences encoding the same amino acid sequence. Included as embodiments of the invention are nucleic acid sequences capable of hybridizing under moderately stringent conditions (e.g., prewashing solution of 5×SSC, 0.5% SDS, 1.0 mM EDTA (pH 8.0) and hybridization conditions of 50° C., 5×SSC, overnight) to the DNA sequences encoding a TSP-30 polynucleotide. The skilled artisan can determine additional combinations of salt and temperature that constitute moderate hybridization stringency (see also, Sambrook, Molecular Cloning: A Laboratory Manual, Cold Spring Harbor Laboratory Press, 1989; Maniatis, Molecular Cloning: A Laboratory Manual, Cold Spring Harbor Laboratory Press, 1982; and Ausubel, Current Protocols in Molecular Biology, Wiley and Sons, 1989 and later versions, which are incorporated herein by reference). Conditions of higher stringency include higher temperatures for hybridization and post-hybridization washes, and/or lower salt concentration. Percent identity of nucleic acids may be determined using, for example, the methods described above for polypeptides, i.e., by methods including visual inspection and/or the use of computer programs such as GAP.

In another aspect, the present invention provides nucleic acids that can be used to produce recombinant TSP-30 polypeptides. Any suitable expression system may be employed for the production of recombinant TSP-30 polypeptides. For example, a recombinant expression vector can be used. In one embodiment, the recombinant expression vector comprises a nucleic acid (e.g., DNA or RNA) encoding a TSP-30 polypeptide operably linked to one or more suitable transcriptional and translational regulatory nucleotide sequences, such as those derived from a mammalian, microbial, viral, or insect gene. A TSP-30 nucleic acid molecule and a regulatory sequence are operably linked when they are present together in an in vitro or in vivo system such that the regulatory sequence can be used to alter the expression of the TSP-30 nucleic acid molecule. One of skill in the art will appreciate that some regulatory sequences (e.g., certain promoter sequences), must be part of the same nucleic acid molecule, in close proximity to, in a particular orientation with respect to, and upstream, downstream, or overlapping with a TSP-30 nucleic acid in order for them to be operably linked. Other regulatory sequences (e.g., certain enhancer elements) can be, for example, located at a distance from the TSP-30 nucleic acid, either upstream or downstream, in any orientation, and/or, in some cases, even on a different nucleic acid than the TSP-30 nucleic acid. Examples of regulatory sequences include transcriptional promoters, operators, enhancers, ribosomal binding sites, internal ribosome entry sites (IRES), and other sequences that affect transcription initiation, elongation or termination, post-transcriptional RNA processing or modification (e.g., splicing, polyadenylation, or other covalent RNA modification), translation initiation, elongation or termination, or other aspects of gene expression. The expression vector can further comprise a polynucleotide encoding a signal peptide. The signal peptide can be, for example, native or heterologous. The polynucleotide encoding the signal peptide can be fused in frame to the TSP-30 polypeptide-encoding polynucleotide so that the TSP-30 polypeptide is initially translated as a fusion protein comprising the signal peptide. A signal peptide is functional in a host cell if it directs the TSP-30 polypeptide to the host cell's secretory apparatus. In one embodiment, a TSP-30 polypeptide comprising a signal peptide is secreted from the host cell. In another embodiment, a TSP-30 polypeptide further comprising one or more transmembrane sequences is inserted into the cytoplasmic membrane of the host cell. Typically, the signal peptide is cleaved from the TSP-30 polypeptide as it transits the secretory apparatus.

Suitable host cells for expression of TSP-30 polypeptides include prokaryotes, yeast, and higher eukaryotic cells, including insect and mammalian cells. Appropriate cloning and expression vectors for use with bacterial, fungal, yeast, insect, and mammalian cellular hosts are described, for example, in Pouwels et al. Cloning Vectors: A Laboratory Manual, Elsevier, New York, 1985.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, TSP-30 polypeptides may include an N-terminal methionine residue to facilitate expression of the recombinant polypeptide in the prokaryotic host cell. The N-terminal Met may be cleaved from the expressed recombinant polypeptide.

Expression vectors for use in prokaryotic host cells generally comprise nucleotide sequences encoding one or more selectable markers. A selectable marker is a polynucleotide that encodes a product that imparts a selectable phenotype to a host cell expressing it. Examples of selectable markers include a polynucleotide encoding a protein that confers antibiotic resistance to the host cell, such that the host cell can grow in the presence of an otherwise toxic concentration of an antibiotic, or a protein that supplies an autotrophic requirement to the host cell, such that the host cell can grow in the absence of a nutrient it would otherwise require in its growth medium. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. In one embodiment, an appropriate promoter and a TSP-30 polynucleotide sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., 1978, Nature 275:615; Goeddel et al., 1979, Nature 281:544), tryptophan (trp) promoter system (Goeddel et al., 1980, Nucl. Acids Res. 8:4057; EP-A-EP-A-36776) and tac promoter (Maniatis, Molecular Cloning: A Laboratory Manual, Cold Spring Harbor Laboratory, p. 412, 1982). A particularly useful prokaryotic host cell expression system employs a phage λ PL promoter and a cI857ts thermolabile repressor sequence. Plasmid vectors available from the American Type Culture Collection which incorporate derivatives of the λ PL promoter include plasmid pHUB2 (resident in E. coli strain JMB9, ATCC 37092) and pPLc28 (resident in E. coli RR1, ATCC 53082).

The stability of TSP-30 polypeptides allows them to be expressed and recovered in active form from prokaryotic expression systems. For example, the cysteine repeat and TSR domains will spontaneously re-fold into an active conformation even after being reduced and boiled in SDS loading buffer. In one embodiment, the TSP-30 polypeptide is renatured in gradually reducing concentrations of urea.

TSP-30 polypeptides may also be expressed in yeast host cells, preferably from the Saccharomyces genus (e.g., S. cerevisiae). Other genera of yeast, such as Pichia or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., 1980, J. Biol. Chem. 255:2073) or other glycolytic enzymes (Hess et al., 1968, J. Adv. Enzyme Reg. 7:149; Holland et al., 1978, Biochem. 17:4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are further described in Hitzeman, EPA-73,657. Another alternative is the glucose-repressible ADH2 promoter described by Russell et al., 1982, J. Biol. Chem. 258:2674, and Beier et al., 1982, Nature 300:724. Shuttle vectors replicable in both yeast and E. coli may be constructed by inserting DNA sequences from pBR322 for selection and replication in E. coli (Amp^{R} gene and origin of replication) into the above-described yeast vectors.

The yeast α-factor leader sequence may be employed to direct secretion of recombinant polypeptides. The α-factor leader sequence is often inserted between the promoter sequence and the structural gene sequence. See, e.g., Kurjan et al., 1982, Cell 30:933; Bitter et al., 1984, Proc. Natl. Acad. Sci. USA 81:5330. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., 1978, Proc. Natl. Acad. Sci. USA 75:1929. The Hinnen et al. protocol selects for Trp⁺transformants in a selective medium, wherein the selective medium consists of 0.67% yeast nitrogen base, 0.5% casamino acids, 2% glucose, 10 μg/ml adenine and 201g/ml uracil.

Yeast host cells transformed by vectors containing an ADH2 promoter sequence may be grown for inducing expression in a “rich” medium. An example of a rich medium is one consisting of 1% yeast extract, 2% peptone, and 1% glucose supplemented with 80 μg/ml adenine and 80 μg/ml uracil. Derepression of the ADH2 promoter occurs when glucose is exhausted from the medium.

Insect host cell culture systems also may be employed to express recombinant TSP polypeptides. Bacculovirus systems for production of heterologous polypeptides in insect cells are reviewed by Luckow and Summers, 1988, Bio/Technology 6:47.

In another embodiment, a mammalian cell is used as a host cell. Examples of suitable mammalian host cell lines include the COS-7 line of monkey kidney cells (ATCC CRL 1651) (Gluzman et al., 1981, Cell 23:175), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al., 1991, EMBO J. 10: 2821. For the production of therapeutic polypeptides it is particularly advantageous to use a mammalian host cell line that has been adapted to grow in media that does not contain animal proteins.

Established methods for introducing DNA into mammalian cells have been described (Kaufman, Large Scale Mammalian Cell Culture, 1990, pp. 15-69). Additional protocols using commercially available reagents, such as Lipofectamine (Gibco/BRL) or Lipofectamine-Plus, can be used to transfect cells (Felgner et al., 1987, Proc. Natl. Acad. Sci. USA 84:7413). In addition, electroporation can be used to transfect mammalian cells using conventional procedures, such as those in Sambrook et al. Molecular Cloning: A Laboratory Manual, 2 ed. Vol. 1-3, Cold Spring Harbor Laboratory Press, 1989. Selection of stable transformants can be performed using methods known in the art, such as, for example, resistance to cytotoxic drugs. Kaufman et al., 1990, Meth. in Enzymology 185:487, describes several selection schemes, such as dihydrofolate reductase (DHFR) resistance. A suitable host strain for DHFR selection can be CHO strain DX-B11, which is deficient in DHFR (Urlaub et al., 1980, Proc. Natl. Acad. Sci. USA 77:4216). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., 1978, Nature 273:113; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534) and the tripartite leader (TPL) and VA gene RNAs from Adenovirus 2 (Gingeras et al., 1982, J. Biol. Chem. 257:13475). The internal ribosome entry site (IRES) sequences of viral origin allows dicistronic mRNAs to be translated efficiently (Oh et al., 1993, Current Opinion in Genetics and Development 3:295; Ramesh et al., 1996, Nucleic Acids Research 24:2697). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., 1997, Biotechniques 22:150, and p2A5I described by Morris et al., Animal Cell Technology, 1997, pp. 529-34.

A useful high expression vector, pCAVNOT, has been described by Mosley et al., 1989, Cell 59:335. Other expression vectors for use in mammalian host cells can be constructed as disclosed by Okayama et al., 1983, Mol. Cell. Biol. 3:280. A useful system for stable high level expression of mammalian cDNAs in C127 murine mammary epithelial cells can be constructed substantially as described by Cosman et al., 1986, Mol. Immunol. 23:935. A useful high expression vector, PMLSV N1/N4, described by Cosman et al., 1984, Nature 312:768, has been deposited as ATCC 39890. Additional useful mammalian expression vectors are known in the art.

In one aspect, the present invention provides a TSP-30 polypeptide comprising a signal peptide or leader sequence Any signal peptide known in the art can be used. In one embodiment, the TSP-30 polypeptide comprises a native TSP-30a, b, c and/or d signal peptide. In another embodiment, the TSP polypeptide comprises a heterologous signal peptide. The choice of signal peptide may depend on factors such as the type of host cells in which the TSP-30 polypeptide is to be produced. Examples of heterologous signal peptides that are functional in mammalian host cells include the signal sequence for interleukin-7 (IL-7) (see U.S. Pat. No. 4,965,195), the signal sequence for interleukin-2 receptor (see Cosman et al., 1984, Nature 312:768), the interleukin-4 receptor signal peptide (see EP 367,566), the type I interleukin-1 receptor signal peptide (see U.S. Pat. No. 4,968,607), and the type II interleukin-1 receptor signal peptide (see EP 460,846).

Using the techniques of recombinant DNA including mutagenesis, directed evolution, and the polymerase chain reaction (PCR) (see, e.g., U.S. Pat. Nos. 6,171,820 and 6,238,884), the skilled artisan can produce DNA sequences that encode TSP-30 polypeptides comprising various additions or substitutions of amino acid residues or sequences, or deletions of terminal or internal residues or sequences, including TSP-30 fragments, variants, derivatives, and fusion polypeptides.

Transgenic animals, including mice, goats, sheep, and pigs, and transgenic plants, including tobacco, tomato, legumes, grasses, and grains, may also be used as bioreactors for the production of TSP-30 polypeptides. In the case of transgenic animals, it is particularly advantageous to construct a chimeric DNA including a TSP-30 coding sequence operably linked to cis-acting regulatory sequences that promote expression of the soluble TSP-30 in milk and/or other body fluids (see, e.g., U.S. Pat. No. 5,843,705; U.S. Pat. No. 5,880,327). Examples of transgenic plant systems suitable for expressing TSP-30 polypeptides include those described U.S. Pat. No. 5,639,947 and U.S. Pat. No. 5,889,189.

The skilled artisan will recognize that the procedure for purifying expressed TSP-30 polypeptides will vary according to the host system employed, and whether or not the recombinant polypeptide is secreted. TSP-30 polypeptides can be purified using methods known in the art, including, for example, one or more concentration, salting-out, ion exchange, hydrophobic interaction, affinity purification, HPLC, or size exclusion chromatography steps. Fusion polypeptides comprising Fc moieties (and multimers formed therefrom) offer the advantage of facile purification by affinity chromatography over Protein A or Protein G columns.

Methods of Detection and Diagnosis

In another aspect, the present invention provides methods of detecting certain cell, tissue, or tumor types. The detection can be done using any method known in the art. For example, a cell, tissue, or tumor type can be detected in a sample by detecting the presence of a TSP-30 polypeptide or polynucleotide in the sample, by determining the absolute amount of a TSP-30 polypeptide or polynucleotide in the sample, or by determining the amount of a TSP-30 polypeptide or polynucleotide relative to the amount of another polypeptide or polynucleotide in the sample, such as the amount of a “housekeeping” polypeptide or polynucleotide (e.g., an actin (for example, a β-actin) or a tubulin (for example, an α-tubulin or a β-tubulin)), wherein the presence, absolute amount, or relative amount of the polypeptide or polynucleotide indicates the presence of the cell, tissue, or tumor type in the sample. In another embodiment, the presence or absence in a sample of a type of a cell, tissue, or tumor can be determined. The determination can be made using any method known in the art. For example, the determination can be made by detecting the presence or absence of a TSP-30 polypeptide or polynucleotide in the sample, determining the absolute amount of the polypeptide or polynucleotide in the sample, or determining the relative amount of the polypeptide or polynucleotice in the sample, such as the amount of a “housekeeping” polypeptide or polynucleotide (e.g., an actin (for example, a β-actin) or a tubulin (for example, an α-tubulin or a β-tubulin), wherein the presence, absence, absolute amount, or relative amount of the polypeptide or polynucleotide indicates the presence or absence of the cell, tissue, or tumor type in the sample.

In another embodiment, the method of detecting a cell, tissue, or tumor type, or determining the presence or absence of a cell, tissue, or tumor type, further comprises the detection of one or more additional markers. See, e.g., Immunocytochemistry in Tumor Diagnosis: Proceedings of the Workshop on Immunocytochemistry in Tumor Diagnosis, Russo, ed., 1985, Martinus Nijhoff; Cellular Cancer Markers, Garrett and Sell, ed.s, 1995, Humana Press; Cell Markers, Jasmin, ed., 1981, S. Karger A G.

In one embodiment, the method comprises contacting the sample, cell, tissue, or tumor with a probe. The probe can be any molecule capable of binding to a TSP-30 polypeptide or polynucleotide, e.g., a polynucleotide, polypeptide, or antibody of the invention. For example, the sample, cell, tissue, or tumor, or an extract thereof, can be contacted with the proble under conditions that allow the probe to bind to a TSP-30 polynucleotide or polypeptide in the cell, tissue, tumor, sample, or extract. Binding of the probe to the TSP-30 polynucleotide or polypeptide can be detected or measured using any method known in the art. For example, the probe can be labeled, e.g., with a radioactive, fluorescent, phosphorescent, colorimetric, enzymatic, magnetic, or radioactive marker, and/or attached to a solid surface, e.g., a bead, particle, or reaction vessel. Alternatively, the probe can be detected by contacting the probe with a secondary probe, that is, a molecule (e.g., a polynucleotide, polypeptide, or antibody) capable of specifically binding to the probe, which itself can be labeled or attached to a solid surface. Similarly, tertiary, quaternary, etc., probes can be used.

The cell, tissue, or tumor can be of any type known to express, or not express, a TSP-30 polypeptide or polynucleotide. Examples of adult human cell and tissue types that express huTSP-30b-derived polypeptides and/or polynucleotides include, for example, ectodermal tissue, for example, tissues of the peripheral nervous system (e.g., skin) and of the central nervous system (e.g., brain and spinal cord), lung, and reproductive tissue (e.g., testis). Examples of fetal human cell and tissue types that express huTSP-30b-derived polypeptides and/or polynucleotides include nervous system tissue (e.g., brain), lung, skeletal muscle, and colon. Examples of tumor-related tissues that express huTSP-30b-derived polypeptides and/or polynucleotides include cancers of the reproductive system (e.g., ovarian endometrial cancer cells, ovarian clear cell carcinomas, and ovarian adenocarcinomas), of the breast (e.g., breast carcinomas), and of the skin (e.g., melanomas).

Examples of adult human cell and tissue types that express huTSP-30c-derived polypeptides and/or polynucleotides include, for example, ectodermal tissues, such as tissues of the nervous system (e.g., brain), tissues of the gastrointestinal tract (e.g., stomach, small intestine, and colon), lung, and placenta. Examples of fetal human tissues that express huTSP-30c-derived polynucleotides and/or polypeptides include ectodermal tissues, such as tissues of the nervous tissue (e.g., brain), and lung. Examples of tumor-related tissues that express huTSP-30c-derived polypeptides and/or polynucleotides include cancers of the reproductive system (e.g., ovarian endometrial cancers).

Examples of adult human cell and tissue types that express huTSP-30d-derived polypeptides and/or polynucleotides include, for example, lung, gastrointestinal tract (e.g., stomach, small intestine, and colon), reproductive tissues (e.g., testis and uterus), the nervous sytem, including the central nervous system (e.g., brain and spinal cord) and the peripheral nervous system (e.g., skin), prostate, and placenta. Examples of fetal human cell and tissue types that express huTSP-30d-derived polypeptides and/or polynucleotides include, for example, lung, skeletal muscle, brain, and colon. Examples of tumor related tissues that express huTSP-30d-derived polypeptides and/or polynucleotides include, for example, tumors of the reproductive system (e.g., ovarian endometerial cancer and ovarian adenocarcinomas), breast (e.g., mammary adenocarcinomas), and lung (e.g., lung carcinomas).

Methods of Treatment

In another aspect, the present invention provides compositions and methods for treating or preventing a condition, illness, injury, infection, or disease in a subject by administering a composition comprising an inhibitor or an activator of a TSP-30 to the subject. The inbibitor or activator can be any type of molecule or substance, for example, a polynucleotide or polypeptide (e.g., a TSP-30 polynucleotide or polypeptide), an antibody or antibody derivative, a polypeptide comprising a soluble fragment of a protein (e.g., a receptor) that binds to a TSP-30 polypeptide, and/or a small molecule that binds to TSP-30 polypeptide or polynucleotide. The condition, illness, infection, or disease can be any known in the art, for example, one relating to inflammation or cancer, and can affect any type of cell, tissue, or organ in the subject, for example, nervous system tissue (including peripheral nervous system tissue (e.g., skin) and central nervous system tissue (e.g., brain and spinal cord), lungs, reproductive tissues (e.g., ovaries, uterus, and testis), breast tissue, the prostate, tissues of the gastrointestinal tract (e.g., stomach, small intestine, and colon), muscle (e.g., skeletal muscle), and placenta.

In one embodiment, the TSP-30 polypeptide or nucleic acid modulates angiogenesis in a subject, tissue, organ, or group of cells. The terms “treat,” “treating,” “treatment,” “therapy,” “therapeutic,” and the like are intended to include, for example, preventative therapy, prophylactic therapy, ameliorative therapy, and curative therapy. A “subject” can be any vertebrate. The subject can be, for example, a fish (e.g., a zebrafish) or a mammal (e.g., a bovine, equine, porcine, ovine, canine, feline, or primate), but preferably is a human.

In another aspect, the compounds, compositions, and methods of the invention are used to modulate angiogenesis, endothelial cell proliferation, migration or morphogenesis, or other TSP-30 mediated response in a subject. The term “TSP-30 mediated response” includes any cellular, physiological, or other biological response that is caused or mediated at least in part by TSP-30a, b, c, and/or d, or which may be modulated by inhibiting TSP-30a, b, c, and/or d, examples of which are provided below.

In one aspect, the present invention provides methods and compositions for modulating (e.g., reducing or increasing) angiogenesis in a subject in need of such treatment. In one embodiment, the subject has a disease, disorder or condition that is caused or exacerbated by angiogenesis. In another embodiment, the subject has a disease, disorder or condition that is alleviated by increased angiogenesis.

Angiogenesis is a multi-step developmental process that results in the formation of new blood vessels off of existing vessels. This spatially and temporally regulated process involves loosening of matrix contacts and support cell interactions in the existing vessels by proteases, followed by coordinated movement, morphological alteration, and proliferation of the smooth muscle and endothelial cells of the existing vessel. The nascent cells then extend into the target tissue followed by cell-cell interactions in which the endothelial cells form tubes that the smooth muscle cells surround. In a coordinated fashion, extracellular matrix proteins of the vessel are secreted and peri-endothelial support cells are recruited to support and maintain structural integrity (see, e.g., Daniel et al., 2000, Ann. Rev. Physiol. 62:649). Angiogenesis plays important roles in both normal and pathological physiology.

Under normal physiological conditions, angiogenesis is involved in fetal and embryonic development, wound healing, organ regeneration, and female reproductive remodeling processes including formation of the endometrium, corpus luteum, and placenta. Angiogenesis is stringently regulated under normal conditions, especially in adult animals, and perturbation of the regulatory controls can lead to pathological angiogenesis.

Pathological angiogenesis has been implicated in the manifestation and/or progression of inflammatory diseases, certain eye disorders, and cancer. In particular, several lines of evidence support the concept that angiogenesis is essential for the growth and persistence of solid tumors and their metastases (see, e.g., Folkman, 1971, N. Engl. J. Med. 285:1182; Folkman et al., 1989, Nature 339:58; Kim et al., 1993, Nature 362:841; Hori et al., 1991, Cancer Res., 51:6180). Angiogenesis inhibitors are therefore useful for the prevention (e.g., treatment of premalignant conditions), intervention (e.g., treatment of small tumors), and regression (e.g., treatment of large tumors) of cancers (see, e.g., Bergers et al., 1999, Science 284:808).

Among the ocular disorders that can be treated according to the present invention are eye diseases characterized by ocular neovascularization including, but not limited to, diabetic retinopathy (a major complication of diabetes), retinopathy of prematurity (a severe complication during the care of premature infants that frequently leads to chronic vision problems and carries a high risk of blindness), neovascular glaucoma, retinoblastoma, retrolental fibroplasia, rubeosis, uveitis, macular degeneration, and corneal graft neovascularization. Other eye inflammatory diseases, ocular tumors, and diseases associated with choroidal or iris neovascularization can also be treated according to the present invention.

The present invention can also be used to treat cell proliferative disorders, including malignant and metastatic conditions such as solid tumors. Solid tumors include both primary and metastatic sarcomas and carcinomas.

The present invention can also be used to treat inflammatory diseases including, but not limited to, arthritis, rheumatism, and psoriasis.

Other diseases and conditions that can be treated according to the present invention include benign tumors and preneoplastic conditions, myocardial angiogenesis, hemophilic joints, scleroderma, vascular adhesions, atherosclerotic plaque neovascularization, telangiectasia, and wound granulation.

In some instances, stimulating angiogenesis may be beneficial (e.g., during tissue or would repair). Accordingly, in another aspect the present invention provides methods and compositions for promoting angiogenesis. Other disease states that can be treated by promoting angiogenesis include coronary or peripheral atherosclerosis and ischemia of a tissue or organ, including the heart, liver, brain, and the like.

In another aspect, the present invention provides compositions that are useful for modulating other cellular or physiological functions. In one embodiment, the composition promotes the growth of fibroblasts, Wilms' tumor cells and/or keratinocytes, inhibits apoptotic activity in embryonic neurons and/or Wilms' tumor cells treated with cisplatin, stimulates the migration of embryonic neurons and/or osteoblasts, promotes the migration of macrophage and/or neutrophils, promotes neurite outgrowth of embryonic neurons, promotes fibroblast-mediated contraction of collagen gels, stimulates the fibrinolytic activity of endothelial cells, enhances the expression of chemokines in urinary tubular epithelial cells, enhances the synthesis of matrix molecules by fibroblasts, stimulates chondrogenesis in micromass culture of chicken limb buds, promotes angiogenesis, inhibits long-term potentiation in the hippocampus, up-regulates epithelial-mesenchymal interactions, promotes tooth germ development, suppresses BMP-2 action, stimulates development of mesenchymal tissues, promotes the formation of nephrons in kidney development, promotes uteric bud branching morphogenesis, promotes endothelial cell proliferation, promotes migration and guidance of embryonic neurons, promotes synapse formation, and/or promotes neural development. In another embodiment, the composition inhibits the growth of fibroblasts, Wilms' tumor cells and/or keratinocytes, promotes apoptotic activity in embryonic neurons and/or Wilms' tumor cells treated with cisplatin, inhibits the migration of embryonic neurons and/or osteoblasts, inhibits the migration of macrophage and/or neutrophils, inhibits neurite outgrowth of embryonic neurons, inhibits fibroblast-mediated contraction of collagen gels, inhibits the fibrinolytic activity of endothelial cells, inhibits the expression of chemokines in urinary tubular epithelial cells, inhibits the synthesis of matrix molecules by fibroblasts, inhibits chondrogenesis in micromass culture of chicken limb buds, inhibits angiogenesis, promotes long-term potentiation in the hippocampus, down-regulates epithelial-mesenchymal interactions, inhibits tooth germ development, promotes BMP-2 action, inhibits development of mesenchymal tissues, inhibits the formation of nephrons in kidney development, inhibits uteric bud branching morphogenesis, inhibits endothelial cell proliferation, inhibits migration and guidance of embryonic neurons, inhibits synapse formation, and/or inhibits neural development.

In other embodiments compositions of the invention are used to treat, for example, diseases, injuries, or conditions of the nervous system, for example, diseases of the central nervous system or peripheral nervous system, such as primary neoplasia, cerebrovascular disease, Parkinson's disease, Alzheimer's disease, and motor neuron disorders.

In other embodiments, compositions of the invention are used to treat, for example, skin disorders, such as cancer, allergic conditions, and inflammatory conditions, e.g., psoriasis), or for wound healing.

In another aspect, the invention provides an antagonist or agonist of TSP-30a, b, c, and/or d. Any composition or method known in the art for antagonizing or agonizing TSP-30a, b, c, and/or d can be used. In one embodiment, the antagonist or agonist is a mutein, derivative, fragment or variant of TSP-30a, b, c, and/or d, e.g., an antagonist that lacks one or more activities of the native protein such that it competes or interferes with the native protein, e.g., by non-productively binding to a TSP-30a, b, c, and/or d receptor or other protein. Examples of other forms of TSP-30a, b, c, and/or d antagonists or agonists include antibodies, antisense nucleic acids, ribozymes, aptamers, and small molecules directed against TSP-30a, b, c, and/or d or a receptor thereof.

The methods and compositions according to the present invention can be tested in in vivo or in vitro models. Any in vivo or in vitro model known in the art can be used. In one embodiment, an animal model (e.g., a non-human primate, dog, cat, ferret, rat, hamster, guinea pig, mouse, or zebrafish model) is used to detect or measure a physiological effect of a method or composition of the invention, e.g., a side effect or other deleterious effect, or a desired prophylactic or therapeutic activity, or to determine an efficacious or optimal therapeutic dosage.

The amount of a particular TSP-30 antagonist or agonist that will be effective in a particular method of treatment depends upon age, type and severity of the condition to be treated, body weight, desired duration of treatment, method of administration, and other parameters. Effective dosages are determined by a physician or other qualified medical professional. Typical effective dosages are about 0.01 mg/kg to about 100 mg/kg body weight. In some preferred embodiments the dosage is about 0.1-50 mg/kg; in some preferred embodiments the dosage is about 0.5-10 mg/kg. The dosage for local administration is typically lower than for systemic administration. In some embodiments a single administration is sufficient; in others multiple doses over one or more days are required.

The TSP-30 antagonist or agonist typically is administered in the form of a pharmaceutical composition comprising one or more pharmacologically acceptable carriers. Pharmaceutically acceptable carriers include diluents, fillers, adjuvants, excipients, and vehicles that are pharmaceutically acceptable for the route of administration, and may be aqueous or oleaginous suspensions formulated using suitable dispersing, wetting, and suspending agents.

Pharmaceutically acceptable carriers are generally sterile and free of pyrogenic agents, and may include water, oils, solvents, salts, sugars and other carbohydrates, emulsifying agents, buffering agents, antimicrobial agents, and chelating agents. The particular pharmaceutically acceptable carrier and the ratio of active compound to carrier are determined by the solubility and chemical properties of the composition, the mode of administration, and standard pharmaceutical practice.

The compositions as described herein may be contained in a vial, bottle, tube, syringe inhaler or other container for single or multiple administrations. Such containers may be made of glass or a polymer material such as polypropylene, polyethylene, or polyvinylchloride, for example. Preferred containers may include a seal, or other closure system, such as a rubber stopper that may be penetrated by a needle in order to withdraw a single dose and then re-seal upon removal of the needle. All such containers for injectable liquids, lyophilized formulations, reconstituted lyophilized formulations or reconstitutable powders for injection known in the art or for the administration of aerosolized compositions are contemplated for use in the presently disclosed compositions and methods.

The TSP-30 antagonists or agonists are administered to the subject in a manner appropriate to the indication, e.g., by intravenous, transdermal, intradermal, intraperitoneal, intramuscular, intranasal, epidural, oral, topical, subcutaneous, intracavity, sustained release from implants, peristaltic routes, or by any other suitable technique. Parenteral administration is preferred.

In certain embodiments of the claimed invention, the treatment further comprises treating the subject with one or more additional agents such as additional anti-cancer (e.g., chemotherapeutic) or anti-inflammatory agents. The additional agent(s) may be administered prior to, concurrently with, or following the administration of the TSP-30 polypeptide or antagonist. The use of more than one agent is particularly advantageous when the subject that is being treated has a solid tumor. In some embodiments of the claimed invention, the treatment further comprises treating the subject with radiation. Radiation, including brachytherapy and teletherapy, may be administered prior to, concurrently with, or following the administration of the second agent(s) and/or TSP-30 antagonist or agonist.

When the subject that is being treated has a solid tumor, the method preferably includes the administration of, in addition to a TSP-30 polypeptide, one or more chemotherapeutic agents selected from the group consisting of alkylating agents, antimetabolites, vinca alkaloids and other plant-derived chemotherapeutics, nitrosoureas, antitumor antibiotics, antitumor enzymes, topoisomerase inhibitors, platinum analogs, adrenocortical suppressants, hormones, hormone agonists and antagonists, antibodies, immunotherapeutics, blood cell factors, radiotherapeutics, and biological response modifiers.

In some preferred embodiments the method includes administration of, in addition to a TSP-30 polypeptide, one or more chemotherapeutic agents selected from the group consisting of cisplatin, cyclophosphamide, mechloretamine, melphalan, bleomycin, carboplatin, fluorouracil, 5-fluorodeoxyuridine, methotrexate, taxol, asparaginase, vincristine, and vinblastine, lymphokines and cytokines such as interleukins, interferons (including alpha, beta, or delta), and TNF, chlorambucil, busulfan, carmustine, lomustine, semustine, streptozocin, dacarbazine, cytarabine, mercaptopurine, thioguanine, vindesine, etoposide, teniposide, dactinomycin, daunorubicin, doxorubicin, bleomycin, plicamycin, mitomycin, L-asparaginase, hydroxyurea, methylhydrazine, mitotane, tamoxifen, and fluoxymesterone.

In some preferred embodiments the method includes administration of, in addition to a TSP-30 polypeptide, one or more chemotherapeutic agents, including various soluble forms thereof, selected from the group consisting of Flt3 ligand, CD40 ligand, interleukin-2, interleukin-12, 4-1BB ligand, anti-4-1BB antibodies, TNF antagonists and TNF receptor antagonists, TRAIL, VEGF antagonists, VEGF receptor (including VEGF-R1 and VEGF-R2, also known as Fltl and Flkl or KDR) antagonists, Tek antagonists, and CD148 (also referred to as DEP-1, ECRTP, and PTPRJ; see Takahashi et al., 1999, J. Am. Soc. Nephrol. 10:2135-45) agonists. In some preferred embodiments the TSP polypeptides of the invention are used as a component of, or in combination with, “metronomic therapy,” such as those described by Browder et al., 2000, Cancer Research 60:1878 and Klement et al., 2000; J. Clin. Invest. 105:R15; see also Barinaga, 2000, Science 288:245).

The polypeptides, compositions, and methods of the present invention can be used as a first line treatment, for the treatment of residual disease following primary therapy, or as an adjunct to other therapies including chemotherapy, surgery, radiation, and other therapeutic methods known in the art.

When the nucleic acid sequences of the present invention are delivered according to the methods disclosed herein, it is advantageous to use a delivery mechanism so that the sequences will be incorporated into a cell for expression. Delivery systems that may advantageously be employed in the contemplated methods include the use of, for example, viral delivery systems such as retroviral and adenoviral vectors, as well as non-viral delivery systems. Such delivery systems are well known by those skilled in the art.

Methods of Screening

The TSP-30a, b, c, and/or d polypeptides, fragments, muteins, derivatives, and conjugates of the invention can be used in a variety of methods of screening to isolate, for example, TSP-30 agonists and antagonists. TSP-30 agonists are compounds that promote the biological activity of TSP-30a, b, c, and/or d, and TSP antagonists are compounds that inhibit the biological activity of TSP-30a, b, c, and/or d. Compounds identified via the following screening assays can be used in compositions and methods for modulating angiogenesis to treat a variety of disease states. The present invention provides methods of screening for compounds that (1) inhibit or increase TSP-30a, b, c, and/or d gene expression in a target tissue or cell, (2) inhibit or increase the interaction of TSP-30a, b, c, and/or d with a receptor or protein; or (3) bind to TSP-30a, b, c, and/or d to inhibit or increase angiogenesis.

Accordingly, the TSP-30 polypeptides of the invention can be used to regulate, influence, and modulate (i.e., increase or decrease) a biological activity associated with TSP-30a, b, c, and/or d.

In one aspect, the present invention provides screening methods that utilize a model animal system. Any model animal system can be used, for example, a rodent (e.g., a mouse, rat, hamster, or guinea pig), a dog, a cat, a ferret, a cow, a goat, a horse, or a fish (e.g., a zebrafish).

The present invention contemplates the use of assays that are designed to identify compounds that inhibit or increase the expression of a gene encoding TSP-30a, b, c, and/or d. Assays may additionally be utilized that identify compounds that bind to TSP-30a, b, c, and/or d gene regulatory sequences (e.g., promoter sequences; see e.g., Platt, 1994, J. Biol. Chem. 269:28558-62), and that may increase or inhibit the level of TSP-30a, b, c, and/or d gene expression. Such an assay can involve, for example, the use of a control system, in which transcription and translation of the TSP-30a, b, c, and/or d or gene occurs, in comparison to a system including a test agent suspected of influencing normal transcription or translation of a TSP-30a, b, c, and/or d gene. For example, one could determine the rate of TSP-30a, b, c, and/or d RNA produced by cardiac cells, and use this to determine if a test agent influences that rate. To assess the influence of a test agent suspected to influence this normal rate of transcription, one would first determine the rate of TSP-30a, b, c, and/or d RNA production in a cardiac cell culture by, for example, Northern Blotting. One could then administer the test agent to a cardiac cell culture under otherwise identical conditions as the control culture. Then the rate of TSP-30a, b, c, and/or d RNA in the culture treated with the test agent could be determined by, for example, Northern Blotting, and compared to the rate of TSP-30a, b, c, and/or d RNA produced by the control culture cells. An increase in the TSP-30a, b, c, and/or d RNA in the cells contacted with the test agent relative to control cells is indicative of a stimulator of TSP-30a, b, c, and/or d gene transcription and/or translation in cardiac cells, while a decrease is indicative of an inhibitor of TSP-30a, b, c, and/or d gene transcription and/or translation in cardiac cells.

There is a variety of other methods that can be used to determine the level of TSP-30a, b, c, and/or d gene expression as well, and may further be used in assays to determine the influence of a test agent on the level of TSP-30a, b, c, and/or d gene expression. For example, RNA from a cell type or tissue known, or suspected, to express the TSP-30a, b, c, and/or d gene, such as cardiac tissue, may be isolated and tested utilizing hybridization or PCR techniques. The isolated cells can be derived from cell culture or from a subject. The analysis of cells taken from culture may be a necessary step in the assessment of cells to be used as part of a cell-cell-based gene therapy technique or, alternatively, to test the effect of compounds on the expression of the TSP-30a, b, c, and/or d gene. Such analyses may reveal both quantitative and qualitative aspects of the expression pattern of the TSP-30a, b, c, and/or d gene, including activation or inactivation of TSP-30a, b, c, and/or d gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the TSP-30a, b, c, and/or d gene nucleic acid segments described above. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such TSP-30a, b, c, and/or d gene expression assays in situ, i.e., directly upon tissue sections (fixed and/or frozen) of subject tissue obtained from, e.g., biopsies or resections, such that no nucleic acid purification is necessary. TSP-30a, b, c, and/or d gene nucleic acid segments described above can be used as probes and/or primers for such in situ procedures (see, for example, Nuovo, 1992, “PCR In Situ Hybridization: Protocols And Applications,” Raven Press, NY).

Compounds identified via assays such as those described herein may be useful, for example, in methods of increasing or inhibiting angiogenesis influenced by the TSP-30a, b, c, and/or d interaction. Such methods are discussed herein.

Alternatively, assay systems may be designed to identify compounds capable of binding the TSP-30a, b, c, and/or d polypeptides of the invention and increasing or inhibiting angiogenesis. Compounds identified may be useful, for example, in increasing or inhibiting the vascularization of target tissues or cells, may be utilized in screens for identifying compounds that disrupt normal TSP-30a, b, c, and/or d interactions, or may in themselves disrupt such interactions.

Any assay known in the art can be used to identify compounds that interact with TSP-30a, b, c, and/or d. In one embodiment, the assay comprises contacting a TSP-30 polypeptide with a test agent under conditions that allow the polypeptide and the test agent to interact. In another embodiment, the polypeptide and the test agent form a complex. In another embodiment, the complex is removed and/or detected. In another embodiment, the TSP-30 polypeptide is anchored, directly or indirectly, to a solid surface and the test agent, and/or complexes of TSP-30 polypeptide and the test agent, anchored to the solid surface at the end of the reaction are detected. In another embodiment, the test agent is anchored, directly or indirectly, to a solid surface and the TSP-30 polypeptide, and/or complexes of TSP-30 polypeptide and test agent, anchored to the solid surface at the end of the reaction are detected. In another embodiment, the TSP-30 polypeptide is anchored to a solid surface, and the test agent, which is not anchored to the solid surface, is labeled, either directly or indirectly. In another embodiment, the TSP-30 polypeptide is anchored, directly or indirectly, to the reaction vessel, and the test agent is anchored, directly or indirectly, to another solid surface, e.g., a bead. Alternatively, the test agent is bound, directly or indirectly, to the reaction vessel and the TSP-30 polypeptide is bound, directly or indirectly, to the bead. The binding of the test agent to the TSP-30 polypeptide can be determined by, for example, determining whether the bead is bound to the reaction vessel.

Microtiter plates can be used as the reaction vessel and/or solid surface. The anchored component can be anchored by any suitable means, e.g., by non-covalent or covalent attachments. Non-covalent attachment may be accomplished by simply coating the solid surface with a solution of the protein and drying. Alternatively, an immobilized antibody, e.g., a monoclonal antibody, specific for the protein to be immobilized can be used to anchor the protein to the solid surface. The surfaces can be prepared in advance and stored.

In one embodiment, a coated surface comprising an anchored first component is contacted with a non-anchored second component. After the reaction is complete, unreacted components are removed (e.g., by washing) under conditions such that any complexes formed will remain anchored to the solid surface. Complexes anchored to the solid surface can be detected using any suitable technique. Where the previously non-anchored component is labeled, the detection of label anchored to the surface indicates that complexes were formed. Where the previously non-anchored component is not labeled, an indirect label can be used to detect complexes anchored to the surface, e.g., using a labeled antibody specific for the previously non-immobilized component (the antibody, in turn, may be directly labeled or indirectly labeled with, for example, a labeled anti-Ig antibody).

Alternatively, a reaction can be conducted in a liquid phase, the reaction products separated from unreacted components, and complexes detected, e.g., using an immobilized antibody specific for a TSP-30a, b, c, and/or d polypeptide or the test agent to anchor any complexes formed in solution, and a labeled antibody specific for the other component of the possible complex to detect anchored complexes.

Those agents identified as binding agents for TSP-30a, b, c, and/or d can be assessed further for their ability to inhibit or promote TSP-30a, b, c, and/or d function, as described below, and thereby increase or decrease, respectively, angiogenesis. Such compounds can then be used therapeutically.

The TSP-30a, b, c, and/or d polypeptides of the present invention can also be used in a screening assay to identify compounds and small molecules that specifically inhibit (antagonize) or enhance (agonize) the disclosed TSP-30a, b, c, and/or d polypeptides. Thus, for example, polypeptides of the invention can be used to identify antagonists and agonists from cells, cell-free preparations, chemical libraries, antibody or antibody fragment or derivative libraries, and natural product mixtures. The antagonists and agonists may be natural or modified substrates, ligands, enzymes, receptors, etc. and the like, of the polypeptides of the instant invention, or may be structural or functional mimetics of the polypeptides. Potential antagonists of the TSP-30a, b, c, and/or d polypeptides of the instant invention may include small molecules, polypeptides, peptides, peptidomimetics, and antibodies that bind to and occupy a binding site of the TSP-30 polypeptides, causing them to be unavailable to interact and therefore preventing their normal ability to modulate angiogenesis. Other potential antagonists are antisense molecules that hybridize to mRNA in vivo and block translation of the mRNA into the polypeptides of the instant invention. Potential agonists include small molecules, polypeptides, peptides, peptidomimetics, and antibodies that bind to the instant TSP-30a, b, c, and/or d polypeptides.

Small molecule agonists and antagonists are usually less than 10 kD molecular weight and may possess a number of physiochemical and pharmacological properties that enhance cell penetration, resist degradation and prolong their physiological half-lives. See Gibbs, 1994, Cell 79:193-98. Antibodies, which include intact molecules as well as fragments such as Fab and F(ab′)₂ fragments, may be used to bind to and inhibit the polypeptides of the instant invention by blocking the commencement of a signaling cascade. It is preferable that the antibodies are humanized, and more preferable that the antibodies are human. The antibodies of the present invention can be prepared by any of a variety of well-known methods.

Alternatively, an antibody can bind to and activate a polypeptide of the instant invention by mimicking the interaction of the polypeptide of the invention with its cognate. One of skill in the art, using the methods and techniques described herein, can determine whether an antibody is an antagonist or agonist.

Specific screening methods are known in the art and many are extensively incorporated in high throughput test systems so that large numbers of test agents can be screened within a short amount of time. The assays can be performed in a variety of formats, including protein-protein binding assays, biochemical screening assays, immunoassays, cell based assays, etc. These assay formats are well known in the art. The screening assays of the present invention are amenable to screening of chemical libraries and are suitable for the identification of small molecule drug candidates, antibodies, peptides and other antagonists and agonists.

One embodiment of the present invention comprises contacting a cell that is responsive to a TSP-30a, b, c, and/or d polypeptide with said polypeptide in the presence of a candidate molecule under conditions where, but for the candidate molecule, said cell would respond to said polypeptide, and determining whether said cell responds to said polypeptide. The response can be determined, for example, via a cell proliferation assay such as, e.g., a cell density assay, corneal pocket assay, or other cell proliferation assay. The response of the cell contacted with the candidate molecule can then be compared with the response of an identical cell that is contacted with said polypeptide in the absence of said candidate molecule. The response that is detected can be any suitable response. For example, the response can be binding of the TSP-30a, b, c, and/or d polypeptide to the cell, or a change in the metabolism, physiology, gene expression, appearance, or behavior of the cell. A decrease in the response indicates the candidate molecule is an antagonist. An increase in the response indicates the candidate molecule is an agonist.

In an assay for compounds that interfere with the activity of a TSP-30a, b, c, and/or d polypeptide the order of addition of reactants can be varied to obtain different information about the compounds being tested. For example, test agents that interfere with the interaction between the TSP-30a, b, c, and/or d polypeptide and the binding partners, e.g., by competition, can be identified by adding the test substance to the reaction mixture prior to, simultaneously with, or after, the TSP-30a, b, c, and/or d polypeptide. Alternatively, test agents that disrupt preformed complexes, e.g., compounds with higher binding constants that displace one of the components from the complex, can be tested by adding the test agent to the reaction mixture after complexes have been formed.

### EXAMPLE 1

This example presents quantitative PCR (TAQMAN®) results for huTSP-30b, huTSP-30c, and huTSP-30d.

RNA samples were obtained from a variety of tissue sources and from cells or tissues treated with a variety of compounds; these RNA samples included commercially available RNA (Ambion, Austin, Tex.; Clontech Laboratories, Palo Alto, Calif.; and Stratagene, La Jolla, Calif.). The RNA samples were DNase treated (part # 1906, Ambion, Austin, Tex.), and reverse transcribed into a population of cDNA molecules using TAQMAN® Reverse Transcription Reagents (part # N808-0234, Applied Biosystems, Foster City, Calif.) according to the manufacturers instructions using random hexamers. Each population of cDNA molecules was placed into specific wells of a multi-well plate at either 5 ng or 20 ng per well and run in triplicate. Pooling was used when same tissue types and stimulation conditions were applied but collected from different donors. Negative control wells were included in each multi-well plate of samples.

Sets of probes and oligonucleotide primers complementary to mRNAs encoding huTSP-30 polypeptides were designed using Primer Express software (Applied Biosystems, Foster City, Calif.) and synthesized, and PCR conditions for these probe/primer sets were optimized to produce a steady and logarithmic increase in PCR product every thermal cycle between approximately cycle 20 and cycle 30. Oligonucleotide primer sets complementary to β-actin were synthesized and PCR

conditions were optimized for these primer sets also.

Multiplex TAQMAN® PCR reactions using both huTSP-30 and β-actin probe/primer sets were set up in 25-microliter volumes with TAQMAN® Universal PCR Master Mix (part # 4304437, Applied Biosystems, Foster City, Calif.) on an Applied Biosystems Prism 7700 Sequence Detection System. Threshold cycle values (CT) were determined using Sequence Detector software version 1.7a (Applied Biosystems, Foster City, Calif.) and transformed to 2E(-dCT) for relative expression comparison of TSP-30s and β-actin.

The primers and probes used were:

For huTSP-30b:

| | | |
|---|---|---|
| TSP-30b-231F: | 5′-GTCCTGCCCACCTGGATACTT-3′ | (SEQ ID NO:56) |
| TSP-30b-311R: | 5′-GCCTCACAGTGCTCGATCTTG-3′ | (SEQ ID NO:57) |
| TSP-30b-265T: | 5′-CCCGACATGAACAAGT-3′ | (SEQ ID NO:58) |
| Amplicon: | 5′-GTCCTGCCCACCTGGATACTTCGACGCCCGCAACCCCGACATGAACAAGT | (SEQ ID NO:59) |
| | GCATCAAATGCAAGATCGAGCACTGTGAGGC-3′ | |

For huTSP-30c:

| | | |
|---|---|---|
| TSP-30c-69F: | 5′-CAACCGATGGAGACGCAGTA-3′ | (SEQ ID NO:60) |
| TSP-30c-136R: | 5′-AAGACAAACAACCCTTGCAAATG-3′ | (SEQ ID NO:61) |
| TSP-30c-91T: | 5′-ATTTGATACATAACTAGCTCGCT-3′ | (SEQ ID NO:62) |
| Amplicon: | 5′-CAACCGATGGAGACGCAGTAAGCGAGCTAGTTATGTATCAAATCCCA | (SEQ ID NO:63) |
| | TTTGCAAGGGTTGTTTGTCTT-3′ | |

For huTSP-30d:

| | | |
|---|---|---|
| TSP-30d-45F: | 5′-GGACATGCTCGCCCTGAA-3′ | (SEQ ID NO:64) |
| TSP-30d-121R: | 5′-AGATGATACAGCCTGTGCAGTTG-3′ | (SEQ ID NO:65) |
| TSP-30d-64T: | 5′-CCACTTGCTTCTTCCTT-3′ | (SEQ ID NO:66) |
| Amplicon: | 5′-GGACATGCTCGCCCTGAACCGAAGGAAGAAGCAAGTGGGCACTGG | (SEQ ID NO:67) |
| | CCTGGGGGGCAACTGCACAGGCTGTATCATCT-3′ | |

Human TSP-30b was highly expressed in adult lung and testis. Lower expression was found in adult brain, spinal cord, and skin. It was also expressed in human fetal lung, skeletal muscle, brain and colon.

Human TSP-30b was expressed by the ovarian endometrioid cancer cell line CRL 11731 (TOV 112D) the ovarian clear cell carcinoma CRL 11730 (TOV-21G), the ovarian adenocarcinoma cell line HTB-75 (CAOV-3), and by the breast carcinoma cell line NCI-AND-RES. Low expression was also found in the melanoma cell line WM-9.

Human TSP-30c was mainly expressed in the fetal and adult brain and lung. It was also expressed in the adult stomach, colon, small intestine and placenta.

Human TSP-30c was expressed by ovarian endometrioid cancer cell line CRL 11731 (TOV 112D).

Human TSP-30d was highly expressed in adult human lung, digestive tract (including stomach, small intestine, and colon), prostate, testis, placenta and uterus, brain, spinal cord and skin. It was also expressed in human fetal lung, skeletal muscle, brain and colon.

Human TSP-30d was expressed by ovarian endometrioid cancer cell line CRL 11731 (TOV 112D) and by the ovarian adenocarcinoma cell line HTB-161 (NIH OVCAR-3). In contrast to TSP-30b, it was expressed by mammary adenocarcinoma cell line HTB-22 (MCF-7), but not NCI/AND-RES. TSP-30d was also expressed in lung carcinoma cell line CCL-185 (A-549).

### EXAMPLE 2

This example demonstrates the expression pattern of drTSP-30a, c and d in 24 hour old zebrafish embryos.

Developing zebrafish embryos were stained essentially as described in Westerfield, 2000, The Zebrafish Book: A Guide for the Laboratory Use of Zebrafish (Danio rerio), 4th ed., University of Oregon Press, Eugene, Oreg. Briefly, embryos were fixed with 4% paraformaldehyde in PBS overnight at 4° C., then washed for five minutes twice in PBS at ambient temperature. The embryos were removed from their chorions using watchmaker's forceps. Embryos were transferred to vials with 100% methanol, which was replaced with fresh methanol after 5 min. The embryos were cooled to −20° C. for at least 30 min., then brought back to ambient temperature and immersed for 5 min. in 50% methanol in PBST (PBS plus 0.1% Tween), then for 5 min. in 30% methanol in PBST. The embryos were then rinsed twice in PBST for 5 min. each. Next, the embryos were fixed for 20 min. in 4% paraformaldehyde in PBS at ambient temperature, then rinsed twice in PBST for 5 min. each.

The fixed embryos then were digested with proteinase K (10 μg/ml in PBST) at ambient temperature for 5 to 12 min. (depending, in part, on the stage; younger stages are more sensitive), then rinsed briefly in PBST and washed for 5 min. in PBST. The embryos were then fixed again using 4% paraformaldehyde in PBS for 20 min. and washed twice as above using PBST.

For the prehybridization, the embryos were transferred in batches up to 40 into small eppendorf tubes (0.8 ml) in approximately 300 μl of HYB* (50% formamide, 5×SSC, 0.1% Tween-20) with an equal volume of HYB+(HYB* with 5 mg/ml torula (yeast) RNA, 50 μg/ml heparin). After incubating for 5 min. at 55° C., HYB* was replaced with an equal volume of HYB+ and prehybridized for 4 hr at 55° C.

RNA probes were prepared according to the Boehringer instructions (Cat. #1175025). The probes were hydrolyzed to an average length of 150-300 nucleotides following the protocol of Cox et al., 1984; Devel. Biol. 101:485-502. After the final precipitation, the hydrolyzed probe were taken up directly in HYB+ and stored at 20° C.

For the hybridization, as much of the preHYB+was removed as possible without letting the embryos touch air. Twenty to 40 μl of fresh HYB+containing 20-100 ng of RNA probe (about 0.5-5.0 ng/μl) were added so that all embryos were covered by the solution. The probe was heated in HYB+for 5 min. at 68° C. before adding to the embryos, then incubated overnight at 55° C.

Probe was then removed.

Embryos were blocked for 1 hour at ambient temperature with PBST plus blocking reagent. Fab-AP as supplied by Boehringer was added at a 1:4000-8000 dilution and shaken for 4 hours at ambient temperature in PBST plus blocking reagent. Embryos were then washed 4 times for 25 min. each with PBST plus blocking reagent, then washed 3 times for 5 min. each in staining buffer.

To stain, embryos were incubated in staining buffer with 4.5 μl NBT and 3.5 μl X-Phosphate (NBT, 75 mg/ml in 70% dimethylformamide; X-Phosphate, 50 mg/ml in dimethylformamide) per ml added and left for at least 30 min. or overnight. The stained embryos were washed in PBS, then dehydrated with 100% methanol twice (10 min. each) and mounted in a 2:1 mixture of benzylbenzoate:benzylalcohol.

The embryos were then fixed in 4% paraformaldehyde at ambient temperature for at least half an hour.

The probes used were full-length drTSP-30a, c and d in either Vector Topo PCR II (Invitrogen) or pMH (Roche Applied Science, Indianapolis, Ind.). The vector was linearized with Bam HI for sense control and Xba I for antisense probe synthesis.

drTSP-30a transcripts were first detected at about 16 hours post-fertilization (all times are for embryos incubated at 28.5° C.) and persisted until about 60 hours post-fertilization. The transcripts were detected in the apical fin fold from about 16 hours, in the pectoral fin bud from about 36 hours, and in certain portions of the central nervous system (CNS). In the CNS, prominent staining was observed in the forebrain. Transcripts also were detected in the spinal cord, the roof and floor plates, and, to a lesser extent, the dorsal diencephelon. Staining also was observed in the otic capsules and the eyes.

drTSP-30c transcripts also were observed in embryos between about 16 and 60 hours post-fertilization. The transcripts were detected in the apical fin fold from about 16 hours, in the pectoral fin bud from about 36 hours, and in certain portions of the CNS. Staining was observed in the CNS, specifically in the spinal cord, the roof and floor plates, and in the dorsal diencephelon, in discreet points along the midline, in the apical region of the fin fold, and in the pectoral fin bud.

drTSP-30d transcripts were observed in the brain (CNS), in discreet points along the midline, and in the dorsal artery.

### EXAMPLE 3

This example demonstrates that the TSP-30 proteins are expressed on the cell surface, undergo post-translational modification, and are naturally shed from the surface of 293 cells.

For the generation of HA (YPYDVPDYA, SEQ ID NO:84) and His6 (HHHHHH, SEQ ID NO:85) tagged proteins, the open reading frames of human TSP-30b, c, and d and of mouse TSP-30c and d were cloned in-frame between the Asp 718 and Not I site of vector pMH (Roche) for HA-tagged constructs and pHM6 (Roche) for His-tagged constructs. These constructs were generated by PCR with the stop codon replaced by a glycine residue. The resulting sequences are shown in SEQ ID NO:68 through SEQ ID NO:77.

Human Embryonic kidney T-cells (293-T) cells were obtained from the ATCC. Cells were plated at a density of 2×106 cells in 6-well tissue culture dishes in DMEM containing 10% fetal calf serum, and standard antibiotics. Each well was transfected with 4 μg of TSP-30 plasmid DNA (HA-tag, His6-tag, or untagged for control), using Lipofectamine (Invitrogen), according to the manufacturer's instructions. Cells were split after 24 hours in 10 cm dishes in DMEM with 2% fetal calf serum. Conditioned media and the cells were harvested at day five for western blots, and day three for flow cytometry.

Cells were detached with PBS-EDTA 5 mM, washed in FACS buffer (PBS, 2% fetal calf serum, 0.01% Na Azide), and incubated with a mouse ascite anti-HA IgG (1/1000), or a mouse anti-polyHis (1/1000) (Sigma) for 1 hour on ice. The cells were washed and incubated with an Alexa-488 conjugated anti-mouse antibody (1/1000, Molecular Probes) for 30 minutes on ice, washed and analyzed with a FACScalibur (Becton).

To concentrate the conditioned medium, 10 μl of Strataclean resin (Stratagene) was mixed with 1 ml of conditioned medium. The protein-linked resin was further mixed with 10 μl of reducing SDS sample buffer (Invitrogen).

Cells were scraped from the culture dish, pelleted, lysed in 1 mL of NP40 lysis buffer (1% NP40 150 mM NaCl 50 mM Tris pH8.0 in H₂O) with complete protease inhibitors (Roche).

Standard western blot techniques were used to investigate the TSP-30s. 5 μl of the conditioned media, and 10 μl of the cell lysate were resolved on a 12% Tris-Glycine gel (Invitrogen). Proteins were transferred on nitrocellulose membranes, blocked in PBS, 0.1% Tween20, 5% dry non-fat milk for 2 hours at room temperature, and incubated over-night with a 1/1000 dilution of a mouse ascite anti-HA antibody (Sigma). The membrane was subsequently washed for 4 times 30 minutes at room temperature, and incubated with a 1/50000 dilution of a peroxidase-conjugated anti-mouse IgG antibody (Sigma) for 2 hours at room temperature. The membranes were further washed for 2 hours at room temperature, and the proteins revealed using an enhanced chemiluminescence kit (Amersham Biosciences).

Human TSP-30b, c, and d and mouse TSP-30c and d showed major shifts of fluorescence when tested for surface expression in 293-T cells, indicating surface expression of the TSP-30s in these cells. This confirms that these TSP-30 proteins are type II transmembrane proteins.

In western blots, human TSP-30b, c, and d and murine TSP-30c and d each showed a band at approximately 30 KDa, corresponding to the predicted weight of the proteins. hTSP-30b showed a major band at 33 KDa, and an additional band at 55 KDa. hSTSP-30c showed a prominent 36 KDa band. hTSP-30d showed multiple post-translationally modified forms, including a 33 kDa, a 36 kDa, and an approximately 64 Kda form. The mouse TSP-30 proteins showed a comparable profile to that of the human forms.

The profile of the conditioned media showed prominent bands at 33 kDa and 36 kDa for hTSP-30b and hTSP-30c, respectively. hTSP-30d showed major bands at approximately 36 and 44 Kda. Mouse forms had a comparable profile to that of the human forms.

For poly-His tagged constructs, a mouse anti-his antibody (1/1000, Sigma) was used. Results were comparable to that obtained with HA-tagged constructs.

Thus, the TSP-30s undergo post-translational modification, and are shed from the surface of transfected 293 cells. This could lead to signaling at sites distant from the sites where TSP-30 producing cells are located.

### EXAMPLE 4

This example demonstrates a role for TSP-30 proteins during embryonic development in zebrafish.

Two splice junction morpholinos (Gene-Tools, Philomath, Oreg.) were designed to target the first and second intron-exon boundary of Danio TSP-30c.

| | | |
|---|---|---|
| MO sj1: | ACAGTTCACTCACCTCTTTTGTTT | (SEQ ID NO:82) |
| MO sj2: | GTGAAAAAATACTGTAGGATCTTA | (SEQ ID NO:83) |

Morpholinos were diluted in sterile water to 1 mM. A Narishige IM300 microinjector was used to deliver 1 nl of a 750 nM or 1 mM morpholino solution to Zebrafish eggs at the 1-cell stage. As a control, a standard morpholino was used. The embryos were maintained at 28.5° C.

The first obvious defect was observed at 36 hours post-fertilization (hpf). Initially, the defect was most visible at the tip of the caudal fin bud, as a defect in the apical fin fold. The defect then extended to affect the entire apical fin fold at 48 hpf. At this time, the embryos showed a massive and general reduction in the thickness of the apical fin fold. The pectoral fin bud also developed normally initially, but the epidermal defect was obvious from 48 hpf. In the most severely affected embryos, the pectoral fin was reduced to a small vestigial remnant or completely absent. No obvious morphological defects were observed elsewhere. This underlines a major role for Danio TSP-30c in the maintenance of the apical fin fold, the apical ectodermal ridge of the pectoral fin bud, and fin formation in Zebrafish.

In order to place TSP-30c in the previously defined molecular pathways controlling fin formation in the Zebrafish, in situ hybridizations were performed with the TSP-30c probes in embryos in which Fgf24, Fgf8, DNp63, and Tbx5 were knocked down with previously published morpholinos:

| | | |
|---|---|---|
| Fgf24: | GACGGCAGAACAGACATCTTGGTCA | (SEQ ID NO:78) |

| | (Fischer et al., 2003, Development | |
|---|---|---|
| | 130:3515-24) | |
| P63: | TGGTCTCCAGGTACAACATATTGGC | (SEQ ID NO:79) |
| | (Lee et al., 2002, Dev Cell. 2:607-16) | |
| Tbx5: | GGTGCTTCACTGTCCGCCATGTCG | (SEQ ID NO:80) |
| | (Ng et al., 2002, Development | |
| | 129:5161-70) | |
| Fgf8: | GAGTCTCATGTTTATAGCCTCAGTA | ((SEQ ID NO:81) |

(Maroon et al., 2002, Development
129:2099-2108)

Whether assayed at 24 hpf, or 48 hpf, a robust signal in the apical fin fold and the pectoral fin bud of Fgf24, Fgf8, DNp63, and Tbx5 morphants was observed. These experiments suggest that TSP-30c does not lie immediately down-stream of these molecules, which have been shown to be essential for fin formation.

It was also determined whether markers of ectodermal or mesenchymal differentiation were maintained in TSP-30c morphants:
Ectoderm: Fgf4, Fgf8, Fgf10, Fgf24
   Msx B/D
   Dlx2, Tbx5
Mesenchyme: Bmp2, Bmp4
   Hox9a, Hox10a
   Shh

The expression of Dlx2 and of Fgf8 was reduced in TSP-30c morphants. All other markers were unmodified between TSP-30c morphants and standard morpholino injected embryos. While Fgf8 has not been shown to be directly involved in fin formation, Fgf8 and Fgf24 in concert determine posterior mesoderm formation.

### EXAMPLE 5

This example demonstrates the effects of an anti-TSP-30c morpholino on cellular proliferation and apoptosis during zebrafish development.

Embryos were treated with the morpholino MO sj1 as described in Example 4.

In order to assess cellular proliferation, embryos were dechorionated by hand between 24 and 48 hours after fertilization, and incubated in 5-bromo-2-deoxyuridine (“Brdu,” Sigma, St. Louis, Mo.) that was diluted to 10 mM in embryo water for 30 minutes at 6° C. The embryos were then washed 6 times in embryo water and incubated for a further 2 hours at 28.5° C. To determine Brdu incorporation, the embryos were first fixed in 4% paraformaldehyde in PBS at 4° C. for 4 hours, washed, incubated for 2 hours at 37° C. in 2N HCl, washed, blocked in PBS-10% goat serum and incubated with an FITC anti-Brdu antibody (1/5, Becton, Franklin Lakes, N.J.).

In order to assess apoptosis, an in-situ terminal deoxynucleotidyl transferase biotin-dUTP nick end labeling (TUNEL) apoptosis detection kit (Roche) was used according to the manufacturer's instructions. Apoptotic cells were revealed using the True Red peroxidase substrate (Vector Labs, Burlingame, Calif.).

Embryos showed patches of unincorporated Brdu cells, distributed mainly around the caudal fin bud, at 24 and 48 hpf. No difference in apoptosis was detected. These results suggest that the defect in the formation of the fin fold seen in TSP-30c morpholino-treated embryos was mainly due to a deficit in proliferation.

The foregoing examples, both actual and prophetic, are non-limiting.

The cited references are incorporated herein in their entireties.

1. An isolated polypeptide comprising amino acid residues 213 through 235 of huTSP-30a 4ex as they are numbered in FIG. 13, wherein said polypeptide has a biological activity of huTSP-30a 4ex.

2. An isolated polypeptide comprising a sequence of at least 15 contiguous amino acid residues of residues 1 through 212 of the sequence of huTSP-30a as they are numbered in FIG. 13, wherein said polypeptide has a biological activity of huTSP-30a 4ex and does not comprise the sequence of amino acid residues 213 through 272 of huTSP-30a as they are numbered in FIG. 13.

3. The isolated polypeptide of claim 2 wherein said polypeptide comprises a sequence of at least 20 contiguous amino acid residues 1 through 212 of the sequence of huTSP30a as they are numbered in FIG. 13.

4. The isolated polypeptide of claim 3 wherein said polypeptide comprises a sequence of at least 25 contiguous amino acid residues 1 through 212 of the sequence of huTSP30a as they are numbered in FIG. 13.

5. An isolated polypeptide comprising a sequence of amino acids that is at least 90% identical to the sequence of huTSP-30a 4ex as they are numbered in FIG. 13, wherein said polypeptide has a biological activity of huTSP-30a 4ex.

6. The isolated polypeptide of claim 5 wherein said polypeptide comprises a sequence of amino acids that is at least 95% identical to the sequence of huTSP-30a 4ex as they are numbered in FIG. 13.

7. The isolated polypeptide of claim 6 wherein said polypeptide comprises a sequence of amino acids that is at least 98% identical to the sequence of huTSP-30a 4ex as they are numbered in FIG. 13.

8. The isolated polypeptide of claim 7 wherein said polypeptide comprises the sequence of amino acids of huTSP-30a 4ex as they are numbered in FIG. 13.

9. An isolated polypeptide comprising a sequence of amino acids that is encoded by a nucleic acid that hybridizes under moderately stringent conditions to a nucleic acid comprising the complement of the highlighted portion of the nucleotide sequence of huTSP-30a 4ex they are numbered in FIG. 2, wherein said polypeptide has a biological activity of huTSP-30a 4ex.

10. An isolated polypeptide comprising an amino acid sequence selected from the group consisting of:
a. residues 225 through 234 of huTSP-30b1 as they are numbered in FIG. 14, and
b. residues 199 through 266 pfhuTSP-30b 4ex as they are numbered in FIG. 14,
wherein said polypeptide has a biological activity of huTSP-30a 4ex.

11. An isolated polypeptide comprising a sequence of at least 15 contiguous amino acid residues of residues 1 through 198 of the sequence of huTSP-30b as they are numbered in FIG. 13, wherein said polypeptide has a biological activity of huTSP-30b 4ex and does not comprise the sequence of amino acid residues 199 through 224 of huTSP-30b as they are numbered in FIG. 14.

12. The isolated polypeptide of claim 11 wherein said polypeptide comprises a sequence of at least 20 contiguous amino acid residues 1 through 198 of the sequence of huTSP30b as they are numbered in FIG. 14.

13. The isolated polypeptide of claim 12 wherein said polypeptide comprises a sequence of at least 25 contiguous amino acid residues 1 through 198 of the sequence of huTSP30b as they are numbered in FIG. 14.

14. An isolated polypeptide comprising a sequence of amino acids that is at least 90% identical to a sequence selected from the group consisting of:
a. huTSP-30b1 as shown in FIG. 14, and
b. huTSP-30b 4ex as shown in FIG. 14,
wherein said polypeptide has a biological activity of huTSP-30 4ex or of huTSP-30b1 and does not consist of a sequence that is identical to huTSP-30b as shown in FIG. 14.

15. The isolated polypeptide of claim 14 wherein said polypeptide comprises a sequence of amino acids that is at least 95% identical to a sequence selected from the group consisting of:
a. huTSP-30b1 as shown in FIG. 14, and
b. huTSP-30b 4ex as shown in FIG. 14.

16. The isolated polypeptide of claim 15 wherein said polypeptide comprises a sequence of amino acids that is at least 98% identical to a sequence selected from the group consisting of:
a. huTSP-30b1 as shown in FIG. 14, and
b. huTSP-30b 4ex as shown in FIG. 14.

17. The isolated polypeptide of claim 16 wherein said polypeptide comprises the sequence of amino acids of huTSP-30b1 or of huTSP-30b 4ex as shown in FIG. 14.

18. An isolated polypeptide comprising a sequence of amino acids that is encoded by a nucleic acid that hybridizes under moderately stringent conditions to a nucleic acid comprising the complement of a nucleotide sequence selected from the group consisting of:
a. the highlighted portion of the nucleotide sequence of huTSP-30b1 as shown in FIG. 2, and
b. the highlighted portion of the nucleotide sequence of huTSP-30b 4ex shown in FIG. 2,
wherein said polypeptide has a biological activity of huTSP-30b1 or of huTSP-30b 4ex.

19. An isolated polypeptide comprising amino acid residues 207 through 242 of huTSP-30c 4ex as they are numbered in FIG. 15, wherein said polypeptide has a biological activity of huTSP-30c 4ex.

20. An isolated polypeptide comprising a sequence of at least 15 contiguous amino acid residues of a sequence selected from the group consisting of:
a. residues 1 through 206 of the sequence of huTSP-30c as they are numbered in FIG. 15,
b. residues 1 through 206 of the sequence of huTSP-30c1 as they are numbered in FIG. 15,
c. residues 1 through 206 of the sequence of huTSP-30c2 as they are numbered in FIG. 15,
d. residues 1 through 206 of the sequence of huTSP-30c3 as they are numbered in FIG. 15, and
e. residues 1 through 206 of the sequence of huTSP-30c 4ex as they are numbered in FIG. 15,
wherein said polypeptide has a biological activity of huTSP-30c, huTSP-30c1, huTSP-30c2, huTSP-30c3, or huTSP-30c 4ex, and does not comprise the sequence of amino acid residues 207 through 243 of huTSP-30c as shown in FIG. 15.

21. The isolated polypeptide of claim 20 wherein said polypeptide comprises a sequence of at least 20 contiguous amino acid residues of a sequence selected from the group consisting of:
a. residues 1 through 206 of the sequence of huTSP-30c as they are numbered in FIG. 15,
b. residues 1 through 206 of the sequence of huTSP-30c1 as they are numbered in FIG. 15,
c. residues 1 through 206 of the sequence of huTSP-30c2 as they are numbered in FIG. 15,
d. residues 1 through 206 of the sequence of huTSP-30c3 as they are numbered in FIG. 15, and
e. residues 1 through 206 of the sequence of huTSP-30c 4ex as they are numbered in FIG. 15.

22. The isolated polypeptide of claim 21 wherein said polypeptide comprises a sequence of at least 25 contiguous amino acid residues of a sequence selected from the group consisting of:
a. residues 1 through 206 of the sequence of huTSP-30c as they are numbered in FIG. 15,
b. residues 1 through 206 of the sequence of huTSP-30c1 as they are numbered in FIG. 15,
c. residues 1 through 206 of the sequence of huTSP-30c2 as they are numbered in FIG. 15,
d. residues 1 through 206 of the sequence of huTSP-30c3 as they are numbered in FIG. 15, and
e. residues 1 through 206 of the sequence of huTSP-30c 4ex as they are numbered in FIG. 15.

23. An isolated polypeptide comprising a sequence of amino acids that is at least 90% identical to a sequence selected from the group consisting of:
a. huTSP-30c3 as shown in FIG. 15, and
b. huTSP-30c 4ex as shown in FIG. 15,
wherein said polypeptide has a biological activity of huTSP-30c 4ex or of huTSP-30c3 and does not consist of a sequence that is identical to huTSP-30c, huTSP-30c1, or huTSP-30c2 as shown in FIG. 14.

24. The isolated polypeptide of claim 23 wherein said polypeptide comprises a sequence of amino acids that is at least 95% identical to a sequence selected from the group consisting of:
a. huTSP-30c3 as shown in FIG. 15, and
b. huTSP-30c 4ex as shown in FIG. 15.

25. The isolated polypeptide of claim 24 wherein said polypeptide comprises a sequence of amino acids that is at least 98% identical to a sequence selected from the group consisting of:
a. huTSP-30c3 as shown in FIG. 15, and
b. huTSP-30c 4ex as shown in FIG. 15.

26. The isolated polypeptide of claim 25 wherein said polypeptide comprises the sequence of amino acids of huTSP-30c3 or of huTSP-30c 4ex as shown in FIG. 15.

27. An isolated polypeptide comprising a sequence of amino acids that is encoded by a nucleic acid that hybridizes under moderately stringent conditions to a nucleic acid comprising the complement of a nucleotide sequence selected from the group consisting of:
a. the highlighted portion of the nucleotide sequence of huTSP-30c3 as shown in FIG. 2, and
b. the highlighted portion of the nucleotide sequence of huTSP-30c 4ex shown in FIG. 2,
wherein said polypeptide has a biological activity of huTSP-30c3 or of huTSP-30c 4ex and does not comprise the sequence of huTSP-30c, huTSP-30c1, huTSP-30c2, huTSP-30c frag1, or huTSP-30c frag2, as shown in FIG. 15.

28. An isolated polypeptide comprising amino acid residues 210 through 292 of huTSP-30d 4ex as they are numbered in FIG. 16, wherein said polypeptide has a biological activity of huTSP-30d 4ex.

29. An isolated polypeptide comprising a sequence of at least 15 contiguous amino acid residues of a sequence selected from the group consisting of:
a. residues 1 through 209 of the sequence of huTSP-30d as they are numbered in FIG. 16,
b. residues 1 through 209 of the sequence of huTSP-30d1 as they are numbered in FIG. 16,
c. residues 1 through 209 of the sequence of huTSP-30d 4ex as they are numbered in FIG. 16,
d. residues 28 through 209 of the sequence of huTSP-30d frag1 as they are numbered in FIG. 16, and
e. residues 33 through 209 of the sequence of huTSP-30d frag2 as they are numbered in FIG. 15,
wherein said polypeptide has a biological activity of huTSP-30d, huTSP-30d1, huTSP-30d 4ex, huTSP-30d frag1, or huTSP-30d frag2 and does not comprise the sequence of amino acid residues 210 through 292 of huTSP-30d as shown in FIG. 16.

30. The isolated polypeptide of claim 29 wherein said polypeptide comprises a sequence of at least 20 contiguous amino acid residues of a sequence selected from the group consisting of:
a. residues 1 through 209 of the sequence of huTSP-30d as they are numbered in FIG. 16,
b. residues 1 through 209 of the sequence of huTSP-30d1 as they are numbered in FIG. 16,
c. residues 1 through 209 of the sequence of huTSP-30d 4ex as they are numbered in FIG. 16,
d. residues 28 through 209 of the sequence of huTSP-30d frag1 as they are numbered in FIG. 16, and
e. residues 33 through 209 of the sequence of huTSP-30d frag2 as they are numbered in FIG. 15.

31. The isolated polypeptide of claim 30 wherein said polypeptide comprises a sequence of at least 25 contiguous amino acid residues of a sequence selected from the group consisting of:
a. residues 1 through 209 of the sequence of huTSP-30d as they are numbered in FIG. 16,
b. residues 1 through 209 of the sequence of huTSP-30d1 as they are numbered in FIG. 16,
c. residues 1 through 209 of the sequence of huTSP-30d 4ex as they are numbered in FIG. 16,
d. residues 28 through 209 of the sequence of huTSP-30d frag1 as they are numbered in FIG. 16, and
e. residues 33 through 209 of the sequence of huTSP-30d frag2 as they are numbered in FIG. 15.

32. An isolated polypeptide comprising a sequence of amino acids that is at least 90% identical to huTSP-30d 4ex as shown in FIG. 16 wherein said polypeptide has a biological activity of huTSP-30d 4ex.

33. The isolated polypeptide of claim 32 wherein said polypeptide comprises a sequence of amino acids that is at least 95% identical to huTSP-30d 4ex as shown in FIG. 16.

34. The isolated polypeptide of claim 33 wherein said polypeptide comprises a sequence of amino acids that is at least 98% identical to huTSP-30d 4ex as shown in FIG. 16.

35. The isolated polypeptide of claim 34 wherein said polypeptide comprises the sequence of amino acids of huTSP-30d 4ex as shown in FIG. 16.

36. An isolated polypeptide comprising a sequence of amino acids that is encoded by a nucleic acid that hybridizes under moderately stringent conditions to a polynucleotide comprising the nucleotide sequence of huTSP-30d 4ex as shown in FIG. 2 wherein said polypeptide has a biological activity of huTSP-30d 4ex and does not comprise the sequence of huTSP-30d, huTSP-30d1, huTSP-30d frag1, or huTSP-30d frag2, as shown in FIG. 16.40.

37. An isolated polypeptide comprising a sequence selected from the group consisting of:
a. a leader sequence as shown in FIG. 13, 14, 15, or 16;
b. an N-terminal heparin binding cluster as shown in FIG. 13, 14, 15, or 16;
c. a cysteine repeat as shown in FIG. 13, 14, 15, or 16; and
d. a thrombospondin repeat as shown in FIG. 13, 14, 15, or 16,
wherein said polypeptide does not comprise a C-terminal heparin binding cluster as shown in FIG. 13, 14, 15, or 16.

38. An isolated polypeptide comprising a sequence selected from the group consisting of:
a. a leader sequence as shown in FIG. 13, 14, 15, or 16;
b. an N-terminal heparin binding cluster as shown in FIG. 13, 14, 15, or 16;
c. a cysteine repeat as shown in FIG. 13, 14, 15, or 16; and
d. a thrombospondin repeat as shown in FIG. 13, 14, 15, or 16,
wherein said polypeptide inhibits huTSP-30a, huTSP-30b, huTSP-30c, or huTSP-30d.

39. An isolated polynucleotide comprising a sequence that encodes the polypeptide of claim 1, 2, 5, 9, 10, 11, 14, 18, 19, 20, 23, 27, 28, 29, 32, 36, 37, or 38.

40. A vector comprising the polynucleotide of claim 39.

41. The vector of claim 40 wherein said vector is an expression vector.

42. A cell comprising the expression vector of claim 41.

43. A method of expressing a polypeptide comprising incubating said cell of claim 42 under conditions that allow expression of said polynucleotide.

44. An isolated antibody that specifically binds to the polypeptide of claim 1, 2, 5, 9, 10, 11, 14, 18, 19, 20, 23, 27, 28, 32, or 36.

45. A pharmaceutical composition comprising the polypeptide of claim 1, 2, 5, 9, 10, 11, 14, 18, 19, 20, 23, 27, 28, 29, 32, 36, 37, or 38 and a pharmaceutically acceptable diluent, buffer, or excipient.

46. A method of treating a condition in a subject comprising administering to said subject an effective amount of the pharmaceutical composition of claim 45.

47. A method of treating a condition in a subject comprising administering to said subject an effective amount of the polynucleotide of claim 39.

48. A method of treating a condition in a subject comprising administering to said subject an effective amount of the antibody of claim 44.

49. A method of determining whether a tissue is cancerous, comprising determining whether said tissue has more of a polypeptide comprising the sequence of huTSP-30a, huTSP-30a 4ex, huTSP-30b, huTSP-30b1, huTSP-30b 4ex, huTSP-30c, huTSP-30c1, huTSP-30c2, huTSP-30c3, huTSP-30c 4ex, huTSP-30c frag1, huTSP-30c frag2, huTSP-30d, huTSP-30d1, huTSP-30d 4ex, huTSP-30d frag1, or huTSP-30d frag2, than a non-cancerous control tissue, wherein more of said polypeptide in said tissue than in said non-cancerous control tissue indicates that said tissue is cancerous.

50. A method of determining whether a tissue is cancerous, comprising determining whether said tissue has more of a polynucleotide that encodes the amino acid sequence of huTSP-30a, huTSP-30a 4ex, huTSP-30b, huTSP-30b1, huTSP-30b 4ex, huTSP-30c, huTSP-30c1, huTSP-30c2, huTSP-30c3, huTSP-30c 4ex, huTSP-30c frag1, huTSP-30c frag2, huTSP-30d, huTSP-30d1, huTSP-30d 4ex, huTSP-30d frag1, or huTSP-30d frag2, than a non-cancerous control tissue, wherein more of said polynucleotide in said tissue than in said non-cancerous control tissue indicates that said tissue is cancerous.


SEQUENCE LISTING (85 sequences)

SEQ ID NO: 1 - DNA, 2133 bp, Homo sapien
  CDS (283)..(1101)
cccggcggctcctggaaccccggttcgcggcgatgccagccaccccagcgaagccgccgcagttcagtgcttggataatttgaaagtacaatagttggtttccctgtccacccgccccacttcgcttgccatcacagcacgcctatcggatgtgagaggagaagtcccgctgctcgggcactgtctatatacgcctaacacctacatatattttaaaaacattaaatataattaacaatcaaaagaaagaggagaaaggaagggaagcattactgggttactatgcacttgcgactgatttcttggctttttatcattttgaactttatggaatacatcggcagccaaaacgcctcccggggaaggcgccagcgaagaatgcatcctaacgttagtcaaggctgccaaggaggctgtgcaacatgctcagattacaatggatgtttgtcatgtaagcccagactattttttgctctggaaagaattggcatgaagcagattggagtatgtctctcttcatgtccaagtggatattatggaactcgatatccagatataaataagtgtacaaaatgcaaagctgactgtgatacctgtttcaacaaaaatttctgcacaaaatgtaaaagtggattttacttacaccttggaaagtgccttgacaattgcccagaagggttggaagccaacaaccatactatggagtgtgtcagtattgtgcactgtgaggtcagtgaatggaatccttggagtccatgcacgaagaagggaaaaacatgtggcttcaaaagagggactgaaacacgggtccgagaaataatacagcatccttcagcaaagggtaacctgtgtcccccaacaaatgagacaagaaagtgtacagtgcaaaggaagaagtgtcagaagggagaacgaggaaaaaaaggaagggagaggaaaagaaaaaaacctaataaaggagaaagtaaagaagcaatacctgacagcaaaagtctggaatccagcaaagaaatcccagagcaacgagaaaacaaacagcagcagaagaagcgaaaagtccaagataaacagaaatcggtatcagtcagcactgtacactagagggttccatgagattattgtagactcatgatgctgctatctcaaccagatgcccaggacaggtgctctagccattaggaccacaaatggacatgtcagttattgctctgtctaaacaacattcccagtagttgctatattcttcatacaagcatagttaacaacaaagagccaaaagatcaaagaagggatactttcagatggttgtcttgtgtgcttctctgcatttttaaaagacaagacattcttgtacatattatcaataggctataagatgtaacaacgaaatgatgacatctggagaagaaacatcttttccttataaaaatgtgttttcaagctgttgttttaagaagcaaaagatagttctgcaaattcaaagatacagtatcccttcaaaacaaataggagttcagggaagagaaacatccttcaaaggacagtgttgttttgaccgggagatctagagagtgctcagaattagggcctggcatttggaatcacaggatttatcatcacagaaacaactgttttaagattagttccatcactctcatcctgtatttttataagaaacacaagagtgcataccagaattgaatataccatatgggattggagaaagacaaatgtggaagaaatcatagagctggagactacttttgtgctttacaaaactgtgaaggattgtggtcacctggaacaggtctccaatctatgttagcactatgtggctcagcctctgttaccccttggattatatatcaacctgtaaacatgtgcctgtaacttacttccaaaaacaaaatcatacttattagaagaaaattctgattttatagaaaaaaaatagagcaaggagaatataacatgtttgcaaagtcatgtgttttctttctcaatgagggaaaaacaattttattacctgcttaatggtccacctggaactaaaagggatactattttctaacaaggtatatctagtaggggagaaagccaccacaataaatatatttgttaatagtttttcaa

SEQ ID NO: 2 - protein, 272 aa, Homo sapien
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 3 - DNA, 1019 bp, Homo sapien
  CDS (283)..(987)
cccggcggctcctggaaccccggttcgcggcgatgccagccaccccagcgaagccgccgcagttcagtgcttggataatttgaaagtacaatagttggtttccctgtccacccgccccacttcgcttgccatcacagcacgcctatcggatgtgagaggagaagtcccgctgctcgggcactgtctatatacgcctaacacctacatatattttaaaaacattaaatataattaacaatcaaaagaaagaggagaaaggaagggaagcattactgggttactatgcacttgcgactgatttcttggctttttatcattttgaactttatggaatacatcggcagccaaaacgcctcccggggaaggcgccagcgaagaatgcatcctaacgttagtcaaggctgccaaggaggctgtgcaacatgctcagattacaatggatgtttgtcatgtaagcccagactattttttgctctggaaagaattggcatgaagcagattggagtatgtctctcttcatgtccaagtggatattatggaactcgatatccagatataaataagtgtacaaaatgcaaagctgactgtgatacctgtttcaacaaaaatttctgcacaaaatgtaaaagtggattttacttacaccttggaaagtgccttgacaattgcccagaagggttggaagccaacaaccatactatggagtgtgtcagtattgtgcactgtgaggtcagtgaatggaatccttggagtccatgcacgaagaagggaaaaacatgtggcttcaaaagagggactgaaacacgggtccgagaaataatacagcatccttcagcaaagggtaacctgtgtcccccaacaaatgagacaagaaagtgtacagtgcaaaggaagaagtgtcagaagggagaacgaggtacaatcataataacaaaatgtgcttgtttgaatcctcataatctgttgcatttttcattttatttcttatgaaacacttggcattatctttcatgcctaat

SEQ ID NO: 4 - protein, 235 aa, Homo sapien [sequence omitted]

SEQ ID NO: 5 - DNA, 705 bp, Homo sapien
  CDS (1)..(705)
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 6 - protein, 234 aa, Homo sapien
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 7 - DNA, 801 bp, Homo sapien
  CDS (1)..(801)
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattag

SEQ ID NO: 8 - protein, 266 aa, Homo sapien
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGGEPQDRHTRLRWERPTGTGRTQITAPQIVSMRLPERPPLAILSCGVHITFPGVLSKEGLPCWVQSQIH

SEQ ID NO: 9 - DNA, 732 bp, Homo sapien
  CDS (1)..(732)
atgcagtttcgccttttctcctttgccctcatcattctgaactgcatggattacagccactgccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacgcggcaaattgttaaaaagccagtgaaagacacaataccgtgtccaaccattgctgaatccaggagatgcaagatgacaatgaggcattgtccaggagggaagagaacaccaaaggcgaaggagaagaggaacaagaaaaagaaaaggaagctgatagaaagggcccaggagcaacacagcgtcttcctagctacagacagagctaaccaataa

SEQ ID NO: 10 - protein, 243 aa, Homo sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTIPCPTIAESRRCKMTMRHCPGGKRTPKAKEKRNKKKKRKLIERAQEQHSVFLATDRANQ

SEQ ID NO: 11 - DNA, 729 bp, Homo sapien
  CDS (1)..(729)
atgcagtttcgccttttctcctttgccctcatcattctgaactgcatggattacagccactgccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtgggatgtgaagttggtcattggagcgaatggggaacttgtagcagaaataatcgcacatgtggatttaaatggggtctggaaaccagaacgcggcaaattgttaaaaagccagtgaaagacacaataccgtgtccaaccattgctgaatccaggagatgcaagatgacaatgaggcattgtccaggagggaagagaacaccaaaggcgaaggagaagaggaacaagaaaaagaaaaggaagctgatagaaagggcccaggagcaacacagcgtcttcctagctacagacagagctaaccaataa

SEQ ID NO: 12 - protein, 242 aa, Homo sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVGCEVGHWSEWGTCSRNNRTCGFKWGLETRTRQIVKKPVKDTIPCPTIAESRRCKMTMRHCPGGKRTPKAKEKRNKKKKRKLIERAQEQHSVFLATDRANQ

SEQ ID NO: 13 - DNA, 732 bp, Homo sapien
  CDS (1)..(732)
atgcagtttcgccttttctcctttgccctcatcattctgaactgcatggattacagccactgccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacgcggcaaattgttaaaaagccagtgaaagacacaatactgtgtccaaccattgctgaatccaggagatgcaagatgacaatgaggcattgtccaggagggaagagaacaccaaaggcgaaggagaagaggaacaagaaaaagaaaaggaagctgatagaaagggcccaggagcaacacagcgtcttcctagctacagacagagctaaccaataa

SEQ ID NO: 14 - protein, 243 aa, Homo sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 15 - DNA, 708 bp, Homo sapien
  CDS (1)..(708)
atgcagtttcgccttttctcctttgccctcatcattctgaactgcatggattacagccactgccaaggcaaccgatggagacgcagtaagcgatgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacgcggcaaattgttaaaaagccagtgaaagacacaataccgtgtccaaccattgctgaatccaggagatgcaagatgacaatgaggcattgtccaggagggaagagaacaccaaaggcgaaggagaagaggaacaagaaaaagaaaaggaagctgatagaaagggcccaggagcaacacagcgtcttcctagctacagacagagctaaccaataa

SEQ ID NO: 16 - protein, 235 aa, Homo sapien
MQFRLFSFALIILNCMDYSHCQGNRWRRSKRCKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTIPCPTIAESRRCKMTMRHCPGGKRTPKAKEKRNKKKKRKLIERAQEQHSVFLATDRANQ

SEQ ID NO: 17 - DNA, 729 bp, Homo sapien
  CDS (1)..(729)
atgcagtttcgccttttctcctttgccctcatcattctgaactgcatggattacagccactgccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacgcggcaaattgttaaaaagccagtgaaagacacaataccgtgtccaaccattgctgaatccaggagatgcaagatgacaatgaggcattgtccaggaggtaggtgtggatataatcactactgtgtgctgtgtgcttcctgtgaattctcccgcttcctaaccaccttcccttccaaaaataagtatttatctttgaaggaaacccaataa

SEQ ID NO: 18 - protein, 242 aa, Homo sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTIPCPTIAESRRCKMTMRHCPGGRCGYNHYCVLCASCEFSRFLTTFPSKNKYLSLKETQ

SEQ ID NO: 19 - DNA, 792 bp, Homo sapien
  CDS (1)..(792)
atgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctag

SEQ ID NO: 20 - protein, 263 aa, Homo sapien
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 21 - DNA, 879 bp, Homo sapien
  CDS (1)..(879)
atgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgagggtgagctgcagcctctgcctccctggggtcagggctcaggcaccagccatgacaggtcctgccacctctgcttcctgcccacggctcaggaagaagcttcagggcaaggccagatggggcacccgcaactcaagacctcatgctgtccaatccagatgccatgtcctggcagagaacagggcctcagaactctgcatagtggaagctggaagcttctgtggtcaacatccttccggttcccccatttcttctga

SEQ ID NO: 22 - protein, 292 aa, Homo sapien
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGELQPLPPWGQGSGTSHDRSCHLCFLPTAQEEASGQGQMGHPQLKTSCCPIQMPCPGREQGLRTLHSGSWKLLWSTSFRFPHFF

SEQ ID NO: 23 - DNA, 1646 bp, Mus sp.
  CDS (486)..(1328)
gaagcagctcgggtctctccagcgccccttgaccatggctgcggtacccacggcgtccgcttccctgcgctcccggggtccctgccacagccgcagccgctgcagcctctgagccccaggggccactgctcgcctggattccgcccgcagccgccgctgctgtgcaaccgaggctaacctgcggccagccaggaggctcctgcaaccttcgctcgcggcgatgacagccaccccagagcagccggctgtgttcggacaatttgagaatgcaattgttggtttcccggtccacccgtcccgcttcgcttgccatcacagcacgcctgttggatctcagtggagaagtcccgctgctctggtttttctactcttcgtatagactcgcctaacacctacatacatatttttctttaaaaaaaaacattaaatataactaacagtgaaaagaaaaaggagagaaaaaagggaaacattacagggttactatgcacttgcgactgatttcttgtttttttatcattttgaactttatggaatacattggcagccaaaacgcctcccgaggaaggcgccagcgaagaatgcatcctaatgtcagtcaaggctgccaaggaggctgtgcaacgtgttcagattacaatggctgtttgtcatgtaagcccagactgttttttgttctggaaaggattggcatgaagcagataggagtgtgtctctcttcgtgtccaagtggatattacggaactcgatatccagatataaataaatgtacaaaatgcaaagttgactgtgatacctgtttcaacaaaaatttctgcacaaagtgtaaaagtggattttacttacaccttggaaagtgccttgacagttgcccagaagggttagaagccaacaatcatactatggaatgtgtcagtattgtacactgtgaggccagtgaatggagtccatggagtccatgtatgaagaaaggaaaaacatgtggcttcaaaagggggactgaaacacgggtccgagatatactacagcatccttcagccaagggtaacctgtgccccccaaccagcgagacaagaacttgtatagtacaaagaaagaagtgttcaaagggagagcgaggaaaaaagggaagagagagaaaacgaaaaaaactgaataaagaagaaagaaaggaaacaagctcctcctctgacagcaaaggtttggagtccagcattgagaccccagaccagcaggaaaacaaagagaggcagcagcagcagaagagaagagcccgagacaagcaacagaaatcgtctatcgctctctattgggaactggagtcaccctagcaggaaggctcagctatctttcccagaggacccaggcaggcctgtgcagaaggagagaatctctgagtgtgtgctacaaactctcatgacccacttagccttctgcaacgacaggagatgtgcagccactctgggtaccatggttccagccctctcaagttcaccgcctcctgaagaaactggactgagtctgccttctacctgtctgtttctggacatcttttcttcaccaactgtactgctcacatgggccacttggcttaaatggttttccacttctcttttaagacataaaagtttgaaatagtgtatacc

SEQ ID NO: 24 - protein, 281 aa, Mus sp.
MHLRLISCFFIILNFMEYIGSQNASRGRRQRRMHPNVSQGCQGGCATCSDYNGCLSCKPRLFFVLERIGMKQIGVCLSSCPSGYYGTRYPDINKCTKCKVDCDTCFNKNFCTKCKSGFYLHLGKCLDSCPEGLEANNHTMECVSIVHCEASEWSPWSPCMKKGKTCGFKRGTETRVRDILQHPSAKGNLCPPTSETRTCIVQRKKCSKGERGKKGRERKRKKLNKEERKETSSSSDSKGLESSIETPDQQENKERQQQQKRRARDKQQKSSIALYWELESP

SEQ ID NO: 25 - DNA, 732 bp, Mus sp.
  CDS (1)..(732)
atgcgtttttgcctcttctcatttgccctcatcattctgaactgtatggattacagccagtgccaaggcaaccgatggagacgcaataagcgagctagttatgtatcaaatcccatttgcaagggttgtttgtcttgttcgaaggacaatggttgcagccgatgtcaacagaagttgttctttttccttcgaagagaaggaatgcgtcagtatggagagtgcctgcattcctgcccatcagggtattatggacaccgagccccagatatgaacagatgtgcacgatgcagaatagaaaactgtgattcttgctttagcaaagacttttgtacgaagtgcaaagtaggcttttatttgcatagaggccgctgctttgatgaatgtccagatggttttgcaccgttagatgagactatggaatgtgtagaaggttgtgaagttggtcattggagcgaatggggaacgtgtagcagaaacaaccgcacgtgtggatttaaatggggtctggaaaccagaacacggcagattgttaaaaagccagcaaaagacacaataccatgtccgaccattgcggagtccaggagatgcaagatggccatgaggcactgtccaggaggaaagagaacaccaaaggcaaaagagaagagaaacaagaagaagaggcggaagctgattgagagagcccaagagcagcacagcgtcttcctcgctacagacagagtgaaccaatag

SEQ ID NO: 26 - protein, 243 aa, Mus sp.
MRFCLFSFALIILNCMDYSQCQGNRWRRNKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLDETMECVEGCEVGHWSEWGTCSRNNRTCGFKWGLETRTRQIVKKPAKDTIPCPTIAESRRCKMAMRHCPGGKRTPKAKEKRNKKKRRKLIERAQEQHSVFLATDRVNQ

SEQ ID NO: 27 - DNA, 798 bp, Mus sp.
  CDS (1)..(798)
atgcggcttgggctgtgcgtggtggccctggttctgagctggacacacatcgccgtgggcagccgggggatcaagggcaagagacagaggcggatcagtgctgaggggagccaagcctgcgccaagggctgtgagctctgttcagaagtcaacggttgcctcaagtgctcgcccaagctcttcattctgctggagaggaacgacatccgccaggtgggcgtctgcctgccgtcctgcccacctggatactttgatgcccgcaaccccgacatgaacaaatgcatcaaatgcaagatcgagcactgtgaggcctgcttcagccacaacttctgcaccaagtgtcaggagggcttgtacttacacaagggccgctgctatccagcctgccctgagggctctacagccgctaacagcaccatggagtgcggcagtcctgcacaatgtgaaatgagcgagtggtccccgtggggaccctgctccaagaagaggaagctgtgcggtttccggaagggatcggaagagcggacacgcagagtgctccatgctcccgggggagaccacaccacctgctccgacaccaaagagacccgcaagtgtaccgtgcgcaggacgccctgcccagaggggcagaagaggaggaaggggggccagggccggagggagaatgccaacaggcatccggccaggaagaacagcaaggagccgggctccaactctcggagacacaaagggcaacagcagccacagccagggacaacagggccactcacatcagtaggacctacctgggcacagtga

SEQ ID NO: 28 - protein, 265 aa, Mus sp.
MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQ

SEQ ID NO: 29 - DNA, 954 bp, Danio rerio
  CDS (1)..(954)
atgcaattgcaactgatctccattgttttgatcttgcattttatggaatacacaaactgtcagcatcacggctccaggcaccggggaaacaaacaggtatcaggcgtgagctcccagggctgccagggaggctgtcagacctgctcggtttacaatggctgtctgacctgcaaacccaagctttttattcacctggagagggacgggatgaggcagatcggagtgtgcctggcctcctgccccaatggtttctacggcacacgttcccccgacaggaacgactgcataaagtgtgggtcagagtgcgactcgtgctttaacaggaacttttgcctgcgctgcagagcaggatcctacttgcacaagggcaagtgcatggagagctgtccagatgggctggtgccgagcgataccaagaaagagtgtgttgctgcatgtccagcgctctgtgacttgtgtcagaacagtgacacgtgtacgaggtgtgtgccgggacacttccttcatgcagggcaatgccatcatgtctgtcctgatgagtttgagccaaatgactcaatggagtgcattccgacagtgcactgtgaagtgagcgagtggagcgagtggggcacctgctcgcgctcgggaaaaacctgtggcttcaaatggggtgaagaaacaaggacccggaaggtcctacagaatccttctccaatggggagcccatgccctccaacttctgaaaagagggaatgctttgtcaaaaagaagagatgtaagccgccgaaaggccagcggcgaggagagaagaaaaaacgctttaacctgcaagagaaagtgactgcagaggcccgtcgagagaggaagagggaacgagagaaggagacaatcgaccgagaggaatccgaaagcaggaacaaaacagagcagcgccgtcgcagggaccagagcagagacgctggaacagtatag

SEQ ID NO: 30 - protein, 317 aa, Danio rerio
MQLQLISIVLILHFMEYTNCQHHGSRHRGNKQVSGVSSQGCQGGCQTCSVYNGCLTCKPKLFIHLERDGMRQIGVCLASCPNGFYGTRSPDRNDCIKCGSECDSCFNRNFCLRCRAGSYLHKGKCMESCPDGLVPSDTKKECVAACPALCDLCQNSDTCTRCVPGHFLHAGQCHHVCPDEFEPNDSMECIPTVHCEVSEWSEWGTCSRSGKTCGFKWGEETRTRKVLQNPSPMGSPCPPTSEKRECFVKKKRCKPPKGQRRGEKKKRFNLQEKVTAEARRERKREREKETIDREESESRNKTEQRRRRDQSRDAGTV

SEQ ID NO: 31 - DNA, 747 bp, Danio rerio
  CDS (1)..(747)
atgcagtttcgcctgttctcatttgcgctgattgtgttgaactgtgtggactacagccactgtcagacgcagcaatcccaccgctggagaagaaacaaaagagctagctatggagcttaccccatctgtaaaggctgctcggtgtgttccaaggacaacgggtgtatgaactgtcagcccaagctcttcctgttcctcaggagggagaggatgaggcagtacggcgaatgtcttcacgcctgtccgtcaggctactacggcatgcgagggacagagatcaacatgtgctccaggtgcaggatagatgattgtgaggcctgcttcagcaaagacttctgcacaaagtgcaagcttggtttttacttgcacaagggtcgttgctttgaaaagtgtccagagggctttgctcccctagaagacaccatggagtgtggagagggttgtgaagtgggccagtggagtgaatgggggacctgcgccagaagaaacaaaacctgtggtttcaagtggggtctggaaaccagaacaaggcatattgtgaagaagccaccaaaggacacaataccatgtccaaccatcgctgaatctaggaggtgcaagatggcaatgcgacactgcagaaaaggaggttcggggaaaggccaccggaccaaagaccagaagaaaaaatccaacaagcagaggctgcgcttgcgggctcagaaccaacacagcgaccacctcgcctccattcgggcaggccagtaa

SEQ ID NO: 32 - protein, 248 aa, Danio rerio
MQFRLFSFALIVLNCVDYSHCQTQQSHRWRRNKRASYGAYPICKGCSVCSKDNGCMNCQPKLFLFLRRERMRQYGECLHACPSGYYGMRGTEINMCSRCRIDDCEACFSKDFCTKCKLGFYLHKGRCFEKCPEGFAPLEDTMECGEGCEVGQWSEWGTCARRNKTCGFKWGLETRTRHIVKKPPKDTIPCPTIAESRRCKMAMRHCRKGGSGKGHRTKDQKKKSNKQRLRLRAQNQHSDHLASIRAGQ

SEQ ID NO: 33 - DNA, 786 bp, Danio rerio
  CDS (1)..(786)
atgcatttgggactgctggcgctggcactggtcttcttcagctccatgggtcacgccgataacctcaaggcctccaaagcaagaagacagagacggataagcactgaagttcctccatcatgttcgaatggatgtgaacactgctcggagtacaacggctgtcttaaatgtcgaccccgactcttcatcttactggagcgaaatgatatccgtcagataggcatttgcctggccgcgtgtcctgttggatattatggcattcgaaatcgggatatgaacaaatgcacacaatgtaaaatagaaaactgtgaggcatgttttagccgaaacttttgcacaaaatgtaaggagggcctgtactcgcatagaggacgatgtttctccagctgtcctgaaggattcaccgtcaacggcaccatggagtgtgtagtccaatgtgatctaagtgagtggagtccatggggcccttgcatgaagaagaacaaaacatgtggctttaaaaagggtaaccagacccgaaccagggagccccttcaggttccaagtcctgccacctccacaggggccgctccagcctcgggctgcgtcccggagatccagacccagagatgtaccgtgcagaaaaagatcccatgcaaaggagaaaataaaaagaaccaacagaaccgtggagaaaatagcaagaaccgcggacgagactctaaggaaagaggaggaaacaagaaaagaaagaacactaaccggtccaccactgtccccaccattacaaccagcatggtgacctaa

SEQ ID NO: 34 - protein, 261 aa, Danio rerio
MHLGLLALALVFFSSMGHADNLKASKARRQRRISTEVPPSCSNGCEHCSEYNGCLKCRPRLFILLERNDIRQIGICLAACPVGYYGIRNRDMNKCTQCKIENCEACFSRNFCTKCKEGLYSHRGRCFSSCPEGFTVNGTMECVVQCDLSEWSPWGPCMKKNKTCGFKKGNQTRTREPLQVPSPATSTGAAPASGCVPEIQTQRCTVQKKIPCKGENKKNQQNRGENSKNRGRDSKERGGNKKRKNTNRSTTVPTITTSMVT

SEQ ID NO: 35 - protein, 51 aa, Homo sapien
DDGWSPWSEWTSCSTSCGNGIQQRGRSCDSLNNRCEGSSVQTRTCHIQECD

SEQ ID NO: 36 - protein, 56 aa, Homo sapien
DGGWSHWSPWSSCSVTCGDGVITRIRLCNSPSPQMNGKPCEGEARETKACKKDACP

SEQ ID NO: 37 - protein, 56 aa, Homo sapien
NGGWGPWSPWDICSVTCGGGVQKRSRLCNNPTPQFGGKDCVGDVTENQICNKQDCP

SEQ ID NO: 38 - protein, 54 aa, Homo sapien
ETCIYSNWSPWSACSSSTCDKGKRMRQRMLKAQLDLSVPCPDTQDFQPCMGPGC

SEQ ID NO: 39 - protein, 56 aa, Homo sapien
STCTMSEWITWSPCSISCGMGMRSRERYVKQFPEDGSVCTLPTEEMEKCTVNEECS

SEQ ID NO: 40 - protein, 54 aa, Homo sapien
PSSCLMTEWGEWDECSATCGMGMKKRHRMIKMNPADGSMCKAETSQAEKCMMPC

SEQ ID NO: 41 - protein, 55 aa, Homo sapien
TIPCLLSPWSEWSDCSVTCGKGMRTRQRMLKSLAELGDCNEDLEQVEKCMLPECP

SEQ ID NO: 42 - protein, 55 aa, Homo sapien
IDCELTEWSQWSECNKSCGKGHVIRTRMIQMEPQFGGAPCPETVQRKKCRIRKCL

SEQ ID NO: 43 - protein, 55 aa, Homo sapien
PGCRMRPWTAWSECTKLCGGGIQERYMTVKKRFKSSQFTSCKDKKEIRACNVHPC

SEQ ID NO: 44 - protein, 54 aa, Homo sapien
DCELSSWSSWTTCDPCQKKRYRYAYLLQPSQFHGEPCNFSDKEVEDCVTNRPCR

SEQ ID NO: 45 - protein, 54 aa, Homo sapien
DCRMSPWSEWSQCDPCLRQMFRSRSIEVFGQFNGKRCTDAVGDRRQCVPTEPCE

SEQ ID NO: 46 - protein, 61 aa, Homo sapien
HCEVSEWNPWSPCTKKGKTCGFKRGTETRVREIIQHPSAKGNLCPPTNETRKCTVQRKKCQ

SEQ ID NO: 47 - protein, 60 aa, Homo sapien
ECELGPWGGWSPCTHNGKTCGSAWGLESRVREAGRAGHEEAATCQVLSESRKCPIQRPCP

SEQ ID NO: 48 - protein, 61 aa, Homo sapien
GCEVGHWSEWGTCSRNNRTCGFKWGLETRTRQIVKKPVKDTIPCPTIAESRRCKMTMRHCP

SEQ ID NO: 49 - protein, 61 aa, Homo sapien
QCEMSEWSPWGPCSKKQQLCGFRRGSEERTRRVLHAPVGDHAACSDTKETRRCTVRRVPCP

SEQ ID NO: 50 - protein, 5 aa, Artificial
  linker
GGGGS

SEQ ID NO: 51 - protein, 12 aa, Artificial
  linker
GKSSGSGSESKS

SEQ ID NO: 52 - protein, 14 aa, Artificial
  linker
GSTSGSGKSSEGKG

SEQ ID NO: 53 - protein, 18 aa, Artificial
  linker
GSTSGSGKSSEGSGSTKG

SEQ ID NO: 54 - protein, 18 aa, Artificial
  linker
GSTSGSGKPGSGEGSTKG

SEQ ID NO: 55 - protein, 14 aa, Artificial
  linker
EGKSSGSGSESKEF

SEQ ID NO: 56 - DNA, 21 bp, Homo sapien
gtcctgcccacctggatactt

SEQ ID NO: 57 - DNA, 21 bp, Homo sapien
gcctcacagtgctcgatcttg

SEQ ID NO: 58 - DNA, 16 bp, Homo sapien
cccgacatgaacaagt

SEQ ID NO: 59 - DNA, 81 bp, Homo sapien
gtcctgcccacctggatacttcgacgcccgcaaccccgacatgaacaagtgcatcaaatgcaagatcgagcactgtgaggc

SEQ ID NO: 60 - DNA, 20 bp, Homo sapien
caaccgatggagacgcagta

SEQ ID NO: 61 - DNA, 23 bp, Homo sapien
aagacaaacaacccttgcaaatg

SEQ ID NO: 62 - DNA, 23 bp, Homo sapien
atttgatacataactagctcgct

SEQ ID NO: 63 - DNA, 68 bp, Homo sapien
caaccgatggagacgcagtaagcgagctagttatgtatcaaatcccatttgcaagggttgtttgtctt

SEQ ID NO: 64 - DNA, 18 bp, Homo sapien
ggacatgctcgccctgaa

SEQ ID NO: 65 - DNA, 23 bp, Homo sapien
agatgatacagcctgtgcagttg

SEQ ID NO: 66 - DNA, 17 bp, Homo sapien
ccacttgcttcttcctt

SEQ ID NO: 67 - DNA, 77 bp, Homo sapien
ggacatgctcgccctgaaccgaaggaagaagcaagtgggcactggcctggggggcaactgcacaggctgtatcatct

SEQ ID NO: 68 - protein, 249 aa, Homo Sapien
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQPGRPLHVYPYDVPDYA

SEQ ID NO: 69 - protein, 246 aa, Homo Sapien
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQPGRPLHVHHHHHH

SEQ ID NO: 70 - protein, 258 aa, Homo Sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQGRPLHVYPYDVPDYA

SEQ ID NO: 71 - protein, 255 aa, Homo Sapien
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTIPCPTIAESRRCKMTMRHCPGGKRTPKAKEKRNKKKKRKLIERAQEQHSVFLATDRANQGRPLHVHHHHHH

SEQ ID NO: 72 - protein, 258 aa, Mus sp.
MRFCLFSFALIILNCMDYSQCQGNRWRRNKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLDETMECVEGCEVGHWSEWGTCSRNNRTCGFKWGLETRTRQIVKKPAKDTIPCPTIAESRRCKMAMRHCPGGKRTPKAKEKRNKKKRRKLIERAQEQHSVFLATDRVNQGRPLHVYPYDVPDYA

SEQ ID NO: 73 - protein, 255 aa, Mus sp.
MRFCLFSFALIILNCMDYSQCQGNRWRRNKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLDETMECVEGCEVGHWSEWGTCSRNNRTCGFKWGLETRTRQIVKKPAKDTIPCPTIAESRRCKMAMRHCPGGKRTPKAKEKRNKKKRRKLIERAQEQHSVFLATDRVNQGRPLHVHHHHHH

SEQ ID NO: 74 - protein, 278 aa, Homo Sapien
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGRPLHVYPYDVPDYA

SEQ ID NO: 75 - protein, 275 aa, Homo Sapien
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGRPLHVHHHHHH

SEQ ID NO: 76 - protein, 280 aa, Mus sp.
MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQGRPLHVYPYDVPDYA

SEQ ID NO: 77 - protein, 277 aa, Mus sp.
MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQGRPLHVHHHHHH

SEQ ID NO: 78 - DNA, 25 bp, Artificial
  Fgf24
gacggcagaacagacatcttggtca

SEQ ID NO: 79 - DNA, 25 bp, Artificial
  P63
tggtctccaggtacaacatattggc

SEQ ID NO: 80 - DNA, 24 bp, Artificial
  Tbx5
ggtgcttcactgtccgccatgtcg

SEQ ID NO: 81 - DNA, 25 bp, Artificial
  Fgf8
gagtctcatgtttatagcctcagta

SEQ ID NO: 82 - DNA, 24 bp, Artificial
  MO sj1
acagttcactcacctcttttgttt

SEQ ID NO: 83 - DNA, 24 bp, Artificial
  MO sj2
gtgaaaaaatactgtaggatctta

SEQ ID NO: 84 - protein, 9 aa, Artificial
  polypeptide
YPYDVPDYA

SEQ ID NO: 85 - protein, 6 aa, Artificial
  polypeptide
HHHHHH